(19) United States
(12) Patent Application Publication
(10) Pub. No.: US 2021/0317540 A1
(43) Pub. Date: Oct. 14, 2021

(54) Microarray-Based Multiplex Fungal Pathogen Analysis

(71) Applicants: Melissa May, Tucson, AZ (US); Frederick H. Eggers, Sahuarita, AZ (US); Kevin M. O'Brien, Sahuarita, AZ (US); Peaches R. Ulrich, Tucson, AZ (US); Benjamin A. Katchman, Tucson, AZ (US); Shayla Freeman, Tucson, AZ (US); Michael E. Hogan, Stony Brook, NJ (US)

(72) Inventors: Melissa May, Tucson, AZ (US); Frederick H. Eggers, Sahuarita, AZ (US); Kevin M. O'Brien, Sahuarita, AZ (US); Peaches R. Ulrich, Tucson, AZ (US); Benjamin A. Katchman, Tucson, AZ (US); Shayla Freeman, Tucson, AZ (US); Michael E. Hogan, Stony Brook, NJ (US)

(73) Assignee: PathogenDx, Inc., Scottsdale, AZ (US)

(21) Appl. No.: 17/356,139

(22) Filed: Jun. 23, 2021

Related U.S. Application Data
(63) Continuation-in-part of application No. 15/916,062, filed on Mar. 8, 2018.
Application No. 15/916,062 is a continuation-in-part of application No. 15/388,561, filed on Dec. 22, 2016, now abandoned.
(60) Provisional application No. 62/271,371, filed on Dec. 28, 2015.

(51) Int. Cl. C12Q 1/6895; C12Q 1/6806; C12Q 1/686; C12Q 1/6816; G01N 21/64

(52) U.S. Cl. CPC C12Q 1/6895; C12Q 1/6806; C12Q 1/686; C12Q 1/6816; C12R 2001/65; G01N 2021/6421; G01N 2021/6439; C12Q 2600/16; G01N 21/6428

(57) ABSTRACT

Provided herein is a method of quantitating a fungus in a plant, plant product or agricultural product. Total nucleic acids are isolated from a sample of the plant or plant product, and an asymmetric PCR amplification reaction is performed using fluorescent labeled primer pairs to obtain fluorescent labeled fungal amplicons. These amplicons are hybridized to fungus specific nucleic acid probes that are attached on a microarray support. The microarray is imaged to detect fluorescent signals from the fluorescent labeled fungal amplicons. The fluorescent signal intensity is correlated to the quantity of fungus.

### CROSS-REFERENCE TO RELATED APPLICATIONS

This application is a continuation-In-part under 35 U.S.C. § 120 of pending application U.S. Ser. No. 15/916,062, filed Mar. 8, 2018, which is a continuation-in-part under 35 U.S.C. § 120 of non-provisional application U.S. Ser. No. 15/388,561, filed Dec. 22, 2016, now abandoned, which claims benefit of priority under 35 U.S.C. § 119(e) of provisional application U.S. Ser. No. 62/271,371, filed Dec. 28, 2015, now abandoned, all of which are hereby incorporated by reference in their entireties.

### BACKGROUND OF THE INVENTION

### Field of the Invention

The present disclosure is in the technical field of DNA based pathogen and plant analysis. More particularly, the present disclosure is in the technical field of pathogen analysis for plant, agriculture, food and water material using a multiplex assay and a 3-dimensional lattice microarray technology for immobilizing nucleic acid probes.

### Description of the Related Art

Several studies have indicated that fungal contaminants are routinely isolated from cannabis plants. In 2000, researchers documented the link between marijuana and heavy contamination by fungal spores (2). In 2017, researchers evaluated 20 cannabis plant samples in the California market contaminated with over 4,000 different fungal taxonomic classifications, including several opportunistic pathogenic fungal agents (Mucor, Aspergillus, Cryptococcus)(3). It is estimated that between 10-20% of cannabis flower fail testing requirements for TYMC (4,5). This calls for superior testing methods for fungal contaminants in plants in general, and cannabis in particular that are rapid and accurate.

Current techniques used to identify microbial pathogens rely upon established clinical microbiology monitoring. Pathogen identification is conducted using standard culture and susceptibility tests. These tests require a substantial investment of time, effort, cost as well as labile products. Current techniques are not ideal for testing large numbers samples. Culture-based testing is fraught with inaccuracies which include both false positives and false negatives, as well as unreliable quantification of colony forming units (CFUs). There are issues with the presence of viable but non-culturable microorganisms which do not show up using conventional culture methods. Certain culture tests are very non-specific in terms of detecting both harmful and harmless species which diminishes the utility of the test to determine if there is a threat present in the sample being tested.

In response to challenges including false positives and culturing of microorganisms, DNA-based diagnostic methods such as polymerase chain reaction (PCR) amplification techniques were developed. To analyze a pathogen using PCR, DNA is extracted from a material prior to analysis, which is a time-consuming and costly step.

In an attempt to eliminate the pre-analysis extraction step of PCR, Colony PCR was developed. Using cells directly from colonies from plates or liquid cultures, Colony PCR allows PCR of bacterial cells without sample preparation. This technique was a partial success but was not as sensitive as culture indicating a possible issue with interference of the PCR by constituents in the specimens. Although this possible interference may not be significant enough to invalidate the utility of the testing performed, such interference can be significant for highly sensitive detection of pathogens for certain types of tests. Consequently, Colony PCR did not eliminate the pre-analysis extraction step for use of PCR, especially for highly sensitive detection of pathogens.

It is known that 16S DNA in bacteria and the ITS2 DNA in yeast or mold can be PCR amplified, and once amplified can be analyzed to provide information about the specific bacteria or specific mold or yeast contamination in or on plant material. Further, for certain samples such as blood, fecal matter and others, PCR may be performed on the DNA in such samples absent any extraction of the DNA. However, for blood it is known that the result of such direct PCR is prone to substantial sample to sample variation due to inhibition by blood analytes. Additionally, attempts to perform direct PCR analysis on plant matter have generally been unsuccessful, due to heavy inhibition of PCR by plant constituents.

Over time, additional methods and techniques were developed to improve on the challenges of timely and specific detection and identification of pathogens. Immuno-assay techniques provide specific analysis. However, the technique is costly in the use of chemical consumables and has a long response time. Optical sensor technologies produce fast real-time detection but such sensor lack identification specificity as they offer a generic detection capability as the pathogen is usually optically similar to its benign background. Quantitative Polymerase Chain Reaction (qPCR) technique is capable of amplification and detection of a DNA sample in less than an hour. However, qPCR is largely limited to the analysis of a single pathogen. Consequently, if many pathogens are to be analyzed concurrently, as is the case with plant, agriculture, food and water material, a relatively large number of individual tests are performed in parallel.

Biological microarrays have become a key mechanism in a wide range of tools used to detect and analyze DNA. Microarray-based detection combines DNA amplification with the broad screening capability of microarray technology. This results in a specific detection and improved rate of process. DNA microarrays can be fabricated with the capacity to interrogate, by hybridization, certain segments of the DNA in bacteria and eukaryotic cells such as yeast and mold. However, processing a large number of PCR reactions for downstream microarray applications is costly and requires highly skilled individuals with complex organizational support. Because of these challenges, microarray techniques have not led to the development of downstream applications.

It is well known that DNA may be linked to a solid support for the purposes of DNA analysis. In those instances, the surface-associated DNA is generally referred to as the “Oligonucleotide probe” (nucleic acid probe, DNA probe) and its cognate partner to which the probe is designed to bind is referred to as the Hybridization Target (DNA Target). In such a device, detection and-or quantitation of the DNA Target is obtained by observing the binding of the Target to the surface bound Probe via duplex formation, a process also called “DNA Hybridization” (Hybridization).

Nucleic acid probe linkage to the solid support may be achieved by non-covalent adsorption of the DNA directly to a surface as occurs when a nucleic acid probe adsorbs to a neutral surface such as cellulose or when a nucleic acid probe adsorbs to cationic surface such as amino-silane coated glass or plastic. Direct, non-covalent adsorption of nucleic acid probes to the support has several limitations. The nucleic acid probe is necessarily placed in direct physical contact with the surface thereby presenting steric constraints to its binding to a DNA Target as the desired (bound) Target-Probe complex is a double helix which can only form by wrapping of the Target DNA strand about the bound Probe DNA: an interaction which is fundamentally inhibited by direct physical adsorption of the nucleic acid probe upon the underlying surface.

Nucleic acid probe linkage may also occur via covalent attachment of the nucleic acid probe to a surface. This can be induced by introduction of a reactive group (such as a primary amine) into the Probe then covalent attachment of the Probe, through the amine, to an amine-reactive moiety placed upon the surface: such as an epoxy group, or an isocyanate group, to form a secondary amine or a urea linkage, respectively. Since DNA is not generally reactive with epoxides or isocyanates or other similar standard nucleophilic substitutions, the DNA Probe must be first chemically modified with “unnatural” ligands such as primary amines or thiols. While such chemistry may be readily implemented during oligonucleotide synthesis, it raises the cost of the DNA Probe by more than a factor of two, due to the cost associated with the modification chemistry. A related UV crosslinking based approach circumvents the need for unnatural base chemistry, wherein Probe DNA can be linked to the surface via direct UV crosslinking of the DNA, mediated by photochemical addition of thymine within the Probe DNA to the amine surface to form a secondary amine adduct. However, the need for high energy UV for efficient crosslinking results in substantial side reactions that can damage the nucleic acid probe beyond use. As is the case for adsorptive linkage, the covalent linkages possible between a modified nucleic acid probe and a reactive surface are very short, in the order of less than 10 rotatable bonds, thereby placing the nucleic acid probe within 2 nm of the underlying surface. Given that a standard nucleic acid probe is >20 bases in length (>10 nm long) a Probe/linker length ratio >10/1 also provides for destabilizing inhibition of the subsequent formation of the desired Target-Probe Duplex.

Previous Attempts at addressing these problems have not met with success. Attachment of nucleic acid probes to surfaces via their entrapment into a 3-Dimensional gel phase such as that created by polymerizing acrylamide and polysaccharides among others have been problematic due to the dense nature of the gel phases. While the pore size (about 10 nm) in these gels permit entrapment and/or attachment of the nucleic acid probes within the gel, the solution-phase DNA Target, which is typically many times longer than the nucleic acid probe, is blocked from penetrating the gel matrix thereby limiting use of these gel phase systems due to poor solution-phase access to the Target DNA.

Thus, the prior art is deficient in methods of DNA based fungal pathogen analysis that interrogates a multiplicity of samples, uses fewer chemical and labile products, reduces processing steps and provides faster results while maintaining accuracy, specificity and reliability. The present invention fulfills this long-standing need and desire in the art.

### SUMMARY OF THE INVENTION

The present invention is directed to a method of quantitating a fungus in a plant. A sample is obtained from the plant, total nucleic acids are isolated, and an asymmetric PCR amplification reaction performed using at least one fluorescent labeled primer pair in which one of the primers is unlabeled, to obtain at least one fluorescent labeled fungal amplicon. The amplicons are hybridized to a plurality of nucleic acid probes each attached at a specific position on a solid microarray support. The sequence in the nucleic acid probes corresponding to sequence determinants in the fungus. The microarray is washed and imaged to detect at least one fluorescent signal from the hybridized fluorescent labeled fungal amplicons. An intensity is the calculated for the fluorescent signal, which correlates with a quantity of fungus in the sample. The present invention is also directed to a related method where total DNA is isolated from the isolated total nucleic acids and the asymmetric PCR amplification reaction performed on the total DNA.

The present invention is also directed to a method of quantitating at least one fungus in an agricultural product. A sample of the agricultural product is obtained, and total nucleic acids are isolated. An asymmetric PCR amplification reaction performed on the total nucleic acid using at least one fluorescent labeled primer pair in which one of the primers is unlabeled, to obtain at least one fluorescent labeled fungal amplicon. The amplicons are hybridized to a plurality of nucleic acid probes each attached at a specific position on a solid microarray support. The sequence in the nucleic acid probes corresponding to sequence determinants in the fungus. The microarray is washed and imaged to detect at least one fluorescent signal from the hybridized fluorescent labeled fungal amplicons. An intensity is the calculated for the fluorescent signal, which correlates with a with a quantity of fungus in the sample. The present invention is also directed to a related method where total DNA is isolated from the isolated total nucleic acids and the asymmetric PCR amplification reaction performed on the total DNA.

The present invention is further directed to a customizable kit comprising the solid support, a plurality of fluorescent labeled bifunctional polymer linkers, solvents and instructions for fabricating the microarray using a plurality of custom designed nucleic acid probes relevant to an end user.

### BRIEF DESCRIPTION OF THE DRAWINGS

These and other features, aspects, and advantages of the embodiments of the present disclosure will become better understood when the following detailed description is read with reference to the accompanying drawings in which like characters represent like parts throughout the drawing, wherein:

FIGS. 1A-1D illustrate a covalent microarray system comprising probes and bifunctional labels printed on an activated surface. FIG. 1A shows the components—unmodified nucleic acid probe, amine-functionalized (NH) bifunctional polymer linker and amine-functionalized (NH) fluorescently labeled bifunctional polymer linker in a solvent comprising water and a high boiling point water-miscible liquid, and a solid support with chemically activatable groups (X). FIG. 1B shows the first step reaction of the bifunctional polymer linker with the chemically activated solid support where the bifunctional polymer linker becomes covalently attached by the amine groups to the chemically activated groups on the solid support. FIG. 1C shows the second step of concentration via evaporation of water from the solvent to increase the concentration of the reactants—nucleic acid probes and bifunctional polymer linker. FIG. 1D shows the third step of UV crosslinking of the nucleic acid probes via thymidine base to the bifunctional polymer linker within evaporated surface, which in some instances also serves to covalently link adjacent bifunctional polymeric linkers together via crosslinking to the nucleic acid Probe.

FIGS. 2A-2D illustrate an adsorptive microarray system comprising probes and bifunctional polymeric linkers. FIG. 2A shows the components; unmodified nucleic acid probe and functionalized (Rₙ) bifunctional polymer linker and similarly functionalized fluorescent labeled bifunctional polymer linker in a solvent comprising water and a high boiling point water-miscible liquid, and a solid support, wherein the Rₙ group is compatible for adsorbing to the solid support surface. FIG. 2B shows the first step adsorption of the bifunctional polymer linker on the solid support where the bifunctional polymer linkers become non-covalently attached by the Rₙ groups to the solid support. FIG. 2C shows the second step of concentration via evaporation of water from the solvent to increase the concentration of the reactants—Nucleic acid probes and bifunctional polymer linker. FIG. 2D shows the third step of UV crosslinking of the nucleic acid probes via thymidine base to the bifunctional polymer linker and other nucleic acid probes within the evaporated surface which in some instances also serves to covalently link adjacent bifunctional polymeric linkers together via crosslinking to the nucleic acid Probe.

FIGS. 3A-3C show experimental data using the covalent microarray system. In this example of the invention the bifunctional polymeric linker was a chemically modified 40 base long oligo deoxythymidine (OligodT) having a Cy5 fluorescent dye attached at its 5′ terminus and an amino group attached at its 3′ terminus, suitable for covalent linkage with a borosilicate glass solid support which had been chemically activated on its surface with epoxysilane. The nucleic acid probes comprised unmodified DNA oligonucleotides, suitable to bind to the solution state target, each oligonucleotide terminated with about 5 to 7 thymidines, to allow for photochemical crosslinking with the thymidines in the top domain of the polymeric (oligodT) linker. FIG. 3A shows an imaged microarray after hybridization and washing, as visualized at 635 nm. The 635 nm image is derived from signals from the (red) CY5 fluor attached to the 5′ terminus of the bifunctional polymer linker (OligodT) which had been introduced during microarray fabrication as a positional marker in each microarray spot. FIG. 3B shows a microarray imaged after hybridization and washing as visualized at 532 nm. The 532 nm image is derived from signals from the (green) CY3 fluor attached to the 5′ terminus of PCR amplified DNA obtained during PCR Reaction #2 of a DNA containing sample. FIG. 3C shows an imaged microarray after hybridization and washing as visualized with both the 532 nm and 635 nm images superimposed. The superimposed images display the utility of parallel attachment of a Cy5-labelled OligodT positional marker relative to the sequence specific binding of the CY3-labelled PCR product.

FIGS. 4A-4B show graphical representation of the position of PCR primers. FIG. 4A is a graphical representation of the position of PCR primers employed within the 16S locus (all bacteria) to be used to PCR amplify unpurified bacterial contamination obtained from Cannabis wash and related plant wash. These PCR primers are used to amplify and dye label DNA from such samples for bacterial analysis via microarray hybridization. FIG. 4B is a graphical representation of the position of PCR primers employed within the stx1 locus (pathogenic E. coli) to be used to PCR amplify unpurified bacterial contamination obtained from Cannabis wash and related plant wash. These PCR primers are used to amplify and dye label DNA from such samples for bacterial analysis via microarray hybridization.

FIGS. 5A-5B show graphical representation of the position of PCR primers. FIG. 5A is a graphical representation of the position of PCR primers employed as a two stage PCR reaction within the stx2 locus (pathogenic E. coli) to be used to PCR amplify unpurified bacterial contamination obtained from Cannabis wash and related plant wash. These PCR primers are used to amplify and dye label DNA from such samples for bacterial analysis via microarray hybridization. FIG. 5B is a graphical representation of the position of PCR primers employed within the invA locus (Salmonella) to be used to PCR amplify unpurified bacterial contamination obtained from Cannabis wash and related plant wash. These PCR primers are used to amplify and dye label DNA from such samples for bacterial analysis via microarray hybridization.

FIG. 6 is a graphical representation of the position of PCR primers employed within the tuf locus (E. coli) to be used to PCR amplify unpurified bacterial contamination obtained from Cannabis wash and related plant wash. These PCR primers are used to amplify and dye label DNA from such samples for bacterial analysis via microarray hybridization.

FIG. 7 is a graphical representation of the position of PCR primers employed within the ITS2 locus (yeast and mold) to be used to PCR amplify unpurified yeast, mold and fungal contamination obtained from Cannabis wash and related plant wash. These PCR primers are used to amplify and dye label DNA from such samples for yeast and mold analysis via microarray hybridization.

FIG. 8 is a graphical representation of the position of PCR primers employed within the ITS1 locus (Cannabis Plant Control) to be used to PCR amplify unpurified DNA obtained from Cannabis wash. These PCR primers are used to amplify and dye label DNA from such samples for DNA analysis via microarray hybridization. This PCR reaction is used to generate an internal plant host control signal, via hybridization, to be used to normalize bacterial, yeast, mold and fungal signals obtained by microarray analysis on the same microarray.

FIG. 9 is a flow diagram illustrating the processing of unpurified Cannabis wash or other surface sampling from Cannabis (and related plant material) so as to PCR amplify the raw Cannabis or related plant material, and then to perform microarray analysis on that material so as to analyze the pathogen complement of those plant samples

FIG. 10 is a representative image of the microarray format used to implement the nucleic acid probes. This representative format comprises 12 microarrays printed on a glass slide, each separated by a Teflon divider (left). Each microarray queries the full pathogen detection panel in quadruplicate. Also, shown is a blow-up (right) of one such microarray for the analysis of pathogens in Cannabis and related plant materials. The Teflon border about each microarray is fit to localize about 50 μL fluid sample for hybridization analysis where fluorescent labeled amplicons and placed onto the microarray for 30 min at room temperature, followed by washing at room temperature then microarray image scanning of the dye-labelled pathogen and host Cannabis DNA.

FIGS. 11A-11B shows representative microarray hybridization data obtained from purified bacterial DNA standards (FIG. 11A) and purified fungal DNA standards (FIG. 11B). In each case, the purified bacterial DNA is PCR amplified as though it were an unpurified DNA, then hybridized on the microarray via the microarray probes described above. The data show that in this microarray format, each of the bacteria can be specifically identified via room temperature hybridization and washing. Similarly, the purified fungal DNA is PCR amplified as though it were an unpurified DNA, then hybridized on the microarray via the microarray probes described above. The data show that in this microarray format, each of the fungal DNAs can be specifically identified via room temperature hybridization and washing.

FIG. 12 shows representative microarray hybridization data obtained from 5 representative raw Cannabis wash samples. In each case, the raw pathogen complement of these 5 samples is PCR amplified, then hybridized on the microarray via the microarray probes described above. The data show that in this microarray format, specific bacterial, yeast, mold and fungal contaminants can be specifically identified via room temperature hybridization and washing.

FIG. 13 shows representative microarray hybridization data obtained from a representative raw Cannabis wash sample compared to a representative (raw) highly characterized, candida samples. In each case, the raw pathogen complement of each sample is PCR amplified, then hybridized on the microarray via the microarray probes described above. The data show that in this microarray format, specific fungal contaminants can be specifically identified via room temperature hybridization and washing on either raw Cannabis wash or cloned fungal sample.

FIG. 14 shows a graphical representation of the position of PCR primers employed in a variation of an embodiment for low level detection of Bacteria in the Family Enterobacteriaceae including E. coli. These PCR primers are used to selectively amplify and dye label DNA from targeted organisms for analysis via microarray hybridization.

FIGS. 15A-15C show graphical representation of microarray hybridization data. FIG. 15A is a graphical representation of microarray hybridization data demonstrating low level detection of E. coli O157:H7 from certified reference material consisting of enumerated colonies of specified bacteria spiked onto Humulus lupulus, (Hop plant). FIG. 15B is a graphical representation of microarray hybridization data demonstrating low level detection of E. coli O1111 from certified reference material consisting of enumerated colonies of specified bacteria spiked onto Humulus lupulus, (Hop plant). FIG. 15C is a graphical representation of microarray hybridization data demonstrating low level detection of Salmonella enterica from certified reference material consisting of enumerated colonies of specified bacteria spiked onto Humulus lupulus, (Hop plant).

FIG. 16 shows diagrams for sample collection and preparation from two methods. Both the tape pull and wash method are used to process samples to provide a solution for microbial detection via microarray analysis.

FIG. 17 shows representative data used for the modification of the Augury Software. A trendline was generated for the mathematical modeling using the CFU and RFU values plotted for high, medium, and low Total Yeast and Mold (TYM) probes for A. nidulans.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements, or steps but not the exclusion of any other item, element or step or group of items, elements, or steps unless the context requires otherwise. Similarly, “another” or “other” may mean at least a second or more of the same or different claim element or components thereof.

In one embodiment of this invention, there is provided a method for quantitating a fungus on a plant, comprising obtaining a sample from the plant; isolating total nucleic acids from the sample; performing on the total nucleic acids an asymmetric PCR amplification reaction using at least one fluorescent labeled primer pair comprising an unlabeled primer, and a fluorescently labeled primer, selective for a target nucleotide sequence in the fungus to generate at least one fluorescent labeled fungal amplicon; hybridizing the fluorescent labeled fungal amplicons to a plurality of nucleic acid probes each having a sequence corresponding to a sequence determinant in the fungus, each of said nucleic acid probes attached at a specific position on a solid microarray support; washing the microarray at least once; imaging the microarray to detect at least one fluorescent signal from the hybridized fluorescent labeled fungal amplicons; and calculating an intensity of the fluorescent signal, said intensity correlating with a quantity of the fungus in the sample, thereby quantitating the fungus on the plant.

In this embodiment, the plant is a cannabis or a hemp or a product produced thereof. For example, the product is an oil such as cannabidiol produced from cannabis and hemp.

In this embodiment, the fungus is any fungus capable of infecting the plants including, but not limited to a yeast, a mold, an Aspergillus species and a Penicillium species.

In this embodiment, an asymmetric PCR amplification is performed on the total nucleic acids using at least one fluorescent labeled primer pair. Each of the fluorescent labeled primer pairs comprise an unlabeled primer, and a fluorescently labeled primer, selective for a target nucleotide sequence in the fungus. In this embodiment, the fluorescently labeled primer in about 4-fold to about 8-fold excess of the unlabeled primer whereby, upon completion of the reaction, the fluorescently labeled amplicon will be primarily single stranded (that is, the reaction is a type of “asymmetric PCR”). In this embodiment, the fluorescent labeled primer pairs have forward (odd numbers) and reverse (even number) sequences shown in SEQ ID: 13-16, 31-34, 133-135 (Table 6). Commercially enzymes and buffers are used in this step. Also, any fluorescent label may be used, including, but not limited to a CY3, a CY5, SYBR Green, a DYLIGHT DY647, a ALEXA FLUOR 647, a DYLIGHT DY547 and a ALEXA FLUOR 550.

Further in this embodiment, the fluorescent labeled fungal amplicons generated are hybridized to a plurality of nucleic acid probes. The nucleic acid probes have a sequence corresponding to sequence determinants in the fungus and have sequences SEQ ID NOS: 86-126 (Table 4) and 136-140 (Table 9). The nucleic acid probes are attached to a solid microarray support. The solid support is any microarray including but not limited to a 3-dimensional lattice microarray.

Further in this embodiment, after hybridization, unhybridized amplicons are removed by washing the microarray. Washed microarrays are imaged to detect a fluorescent signal corresponding to the fluorescent labeled fungal amplicons. Further in this embodiment, an intensity for the fluorescent signal is calculated. The calculated intensity is correlated with the number of fungus specific genomes in the sample, thereby quantitating the fungus in the sample. Based on analysis of fungus-free samples, an experimentally determined intensity threshold is established for the hybridization to each probe on the microarray, such that a fluorescent intensity above that threshold signifies the presence of fungus, while fluorescence intensities below the threshold signifies that fungus was not detected. Also, the fluorescence intensity correlates with a quantity of the fungus in the sample.

Further to this embodiment, the method comprises isolating total DNA after the isolating step and further performing the asymmetric PCR amplification on the total DNA as described above.

In another embodiment of this invention, there is provided a method for quantitating at least one fungus in an agricultural product, comprising obtaining a sample of the agricultural product; isolating total nucleic acids from the sample; performing on the total nucleic acids an asymmetric PCR amplification reaction using at least one fluorescent labeled primer pair comprising an unlabeled primer, and a fluorescently labeled primer, selective for a target nucleotide sequence in the at least one fungus to generate at least one fluorescent labeled fungal amplicon; hybridizing the fluorescent labeled fungal amplicons to a plurality of nucleic acid probes each having a sequence corresponding to a sequence determinant in the fungus, each of said nucleic acid probes attached at a specific position on a solid microarray support; washing the microarray at least once; imaging the microarray to detect at least one fluorescent signal from the hybridized fluorescent labeled fungal amplicons, and calculating an intensity of the fluorescent signal, the intensity correlating with a quantity of the fungus in the sample, thereby quantitating the at least one fungus in the agricultural product.

In this embodiment, the plant is a cannabis or a hemp or a product produced thereof. For example, the product is an oil such as cannabidiol produced from cannabis and hemp.

In this embodiment, the fungus is any fungus capable of infecting the plants including, but not limited to a yeast, a mold, an Aspergillus species and a Penicillium species.

In this embodiment, an asymmetric PCR amplification is performed on the total nucleic acids using at least one fluorescent labeled primer pair. Each of the fluorescent labeled primer pairs comprise an unlabeled primer, and a fluorescently labeled primer, selective for a target nucleotide sequence in the fungus. In this embodiment, the fluorescently labeled primer in about 4-fold to about 8-fold excess of the unlabeled primer whereby, upon completion of the reaction, the fluorescently labeled amplicon will be primarily single stranded (that is, the reaction is a type of “asymmetric PCR”). In this embodiment, the fluorescent labeled primer pairs have forward (odd numbers) and reverse (even number) sequences shown in SEQ ID: 13-16, 31-34, 133-135 (Table 6). Commercially enzymes and buffers are used in this step. Also, any fluorescent label may be used, including, but not limited to a CY3, a CY5, SYBR Green, a DYLIGHT DY647, a ALEXA FLUOR 647, a DYLIGHT DY547 and a ALEXA FLUOR 550.

Further in this embodiment, the fluorescent labeled fungal amplicons generated are hybridized to a plurality of nucleic acid probes. The nucleic acid probes have a sequence corresponding to sequence determinants in the fungus and have sequences SEQ ID NOS: 86-126 (Table 4) and 136-140 (Table 9). The nucleic acid probes are attached to a solid microarray support. The solid support is any microarray including but not limited to a 3-dimensional lattice microarray.

Further in this embodiment, after hybridization, unhybridized amplicons are removed by washing the microarray. Washed microarrays are imaged to detect a fluorescent signal corresponding to the fluorescent labeled fungal amplicons. Further in this embodiment, an intensity for the fluorescent signal is calculated. The calculated intensity is correlated with the number of fungus specific genomes in the sample, thereby quantitating the at least one fungus in the agricultural product. Based on analysis of fungus-free samples, an experimentally determined intensity threshold is established for the hybridization to each probe on the microarray, such that a fluorescent intensity above that threshold signifies the presence of fungus, while fluorescence intensities below the threshold signifies that fungus was not detected. Also, the fluorescence intensity correlates with a quantity of the fungus in the sample.

Further to this embodiment, the method comprises isolating total DNA after the isolating step and further performing the asymmetric PCR amplification on the total DNA as described above.

Described herein is a method for detecting a fungus in a plant sample such as for example a cannabis, or a plant product such as for example a cannabidiol. Total nucleic acids or total DNA is isolated, and an asymmetric PCR amplification reaction performed to generate fluorescent labeled fungal amplicons. The fluorescent labeled fungal amplicons are hybridized to nucleic acid probes attached to a microarray. This method allows positive hybridization signals to be validated on each sample tested based on internal “mismatched” and “sequence specific” controls. The method steps may be performed concurrently, performed in a single assay, which is beneficial since it enables streamlined detection of fungus in a single assay. The method may be employed to detect any fungus in the plant or plant product.

In the embodiments described above, the microarray is made of any suitable material known in the art including but not limited to borosilicate glass, a thermoplastic acrylic resin (e.g., poly(methyl methacrylate-VSUVT) a cycloolefin polymer (e.g. ZEONOR 1060R), a metal including, but not limited to gold and platinum, a plastic including, but not limited to polyethylene terephthalate, polycarbonate, nylon, a ceramic including, but not limited to TiO₂, and Indium tin oxide (ITO) and engineered carbon surfaces including, but not limited to graphene. A combination of these materials may also be used. The solid support has a front surface and a back surface and is activated on the front surface by chemically activatable groups for attachment of the nucleic acid probes. In this embodiment, the chemically activatable groups include but are not limited to epoxysilane, isocyanate, succinimide, carbodiimide, aldehyde and maleimide. These materials are well known in the art and one of ordinary skill in this art would be able to readily functionalize any of these supports as desired. In a preferred embodiment, the solid support is epoxysilane functionalized borosilicate glass support.

The nucleic acid probes are attached either directly to the microarray support, or indirectly attached to the support using bifunctional polymer linkers. In this embodiment, the bifunctional polymer linker has a top domain and a bottom end. On the bottom end is attached a first reactive moiety that allows covalent attachment to the chemically activatable groups in the solid support. Examples of first reactive moieties include but are not limited to an amine group, a thiol group and an aldehyde group. In one aspect the first reactive moiety is an amine group. On the top domain of the bifunctional polymer linker is provided a second reactive moiety that allows covalent attachment to the oligonucleotide probe. Examples of second reactive moieties include but are not limited to nucleotide bases like thymidine, adenine, guanine, cytidine, uracil and bromodeoxyuridine and amino acid like cysteine, phenylalanine, tyrosine glycine, serine, tryptophan, cystine, methionine, histidine, arginine and lysine. The bifunctional polymer linker may be an oligonucleotide such as OLIGOdT, an amino polysaccharide such as chitosan, a polyamine such as spermine, spermidine, cadaverine and putrescine, a polyamino acid, with a lysine or histidine, or any other polymeric compounds with dual functional groups which can be attached to the chemically activatable solid support on the bottom end, and the nucleic acid probes on the top domain. Preferably, the bifunctional polymer linker is OLIGOdT having an amine group at the 5′ end.

In this embodiment, the bifunctional polymer linker may be unmodified with a fluorescent label. Alternatively, the bifunctional polymer linker has a fluorescent label attached covalently to the top domain, the bottom end, or internally. The second fluorescent label is different from the fluorescent label in the fluorescent labeled primers. Having a fluorescent label (fluorescent tag) attached to the bifunctional polymer linker is beneficial since it allows the user to image and detect the position of the individual nucleic acid probes (“spot”) printed on the microarray. By using two different fluorescent labels, one for the bifunctional polymer linker and the second for the amplicons generated from the fungal DNA being queried, the user can obtain a superimposed image that allows parallel detection of those nucleic acid probes which have been hybridized with amplicons. This is advantageous since it helps in identifying the fungus comprised in the sample using suitable computer and software, assisted by a database correlating nucleic acid probe sequence and microarray location of this sequence with a known DNA signature in fungi. Examples of fluorescent labels include, but are not limited to CY5, DYLIGHT DY647, ALEXA FLUOR 647, CY3, DYLIGHT DY547, or ALEXA FLUOR 550. The fluorescent labels may be attached to any reactive group including but not limited to, amine, thiol, aldehyde, sugar amido and carboxy on the bifunctional polymer linker. In one aspect, the bifunctional polymer linker is CY5-labeled OLIGOdT having an amino group attached at its 3′terminus for covalent attachment to an activated surface on the solid support.

Further in this embodiment, when the bifunctional polymer linker is also fluorescently labeled a second fluorescent signal image is detected in the imaging step. Superimposing the first fluorescent signal image and second fluorescent signal image allows identification of the fungus by comparing the sequence of the nucleic acid probe at one or more superimposed signal positions on the microarray with a database of signature sequence determinants for a plurality of fungal DNA. This embodiment is particularly beneficial since it allows identification of more than one type of fungus in a single assay.

QuantX TYM enables quantitating fungus in plants or plant products. The microarray has the capacity to test for multiple fungus and/or multiple plants and/or plant products in parallel. The testing may be performed in triplicate along with a panel of controls as needed, enabling rapid and reliable quantitation of fungus from multiple plant samples.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Fabrication of 3-Dimensional Lattice Microarray Systems

The present invention teaches a way to link a nucleic acid probe to a solid support surface via the use of a bifunctional polymeric linker. The nucleic acid probe can be a PCR amplicon, synthetic oligonucleotides, isothermal amplification products, plasmids or genomic DNA fragment in a single stranded or double stranded form. The invention can be sub-divided into two classes, based on the nature of the underlying surface to which the nucleic acid probe would be linked.

Covalent Microarray System with Activated Solid Support.

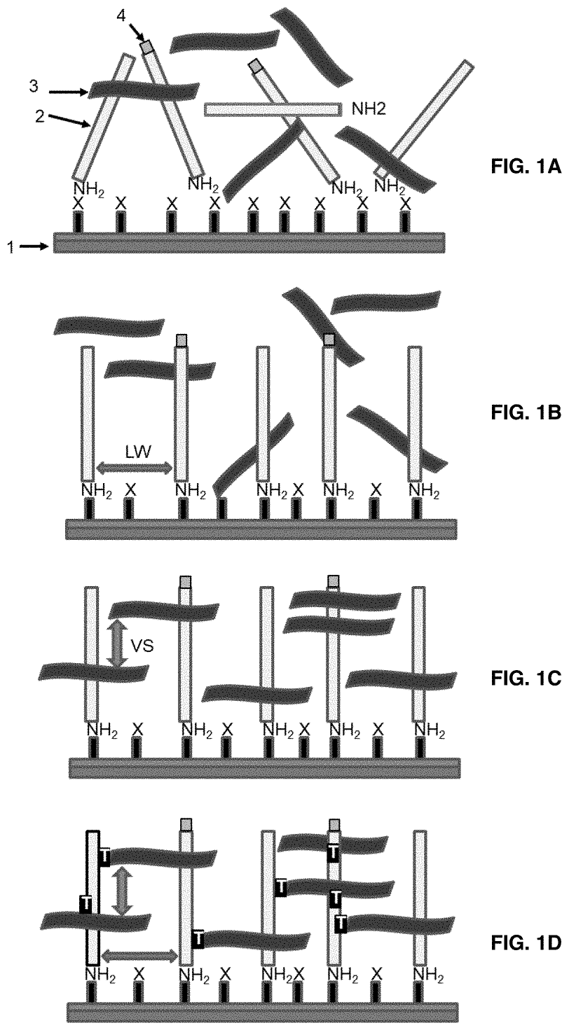

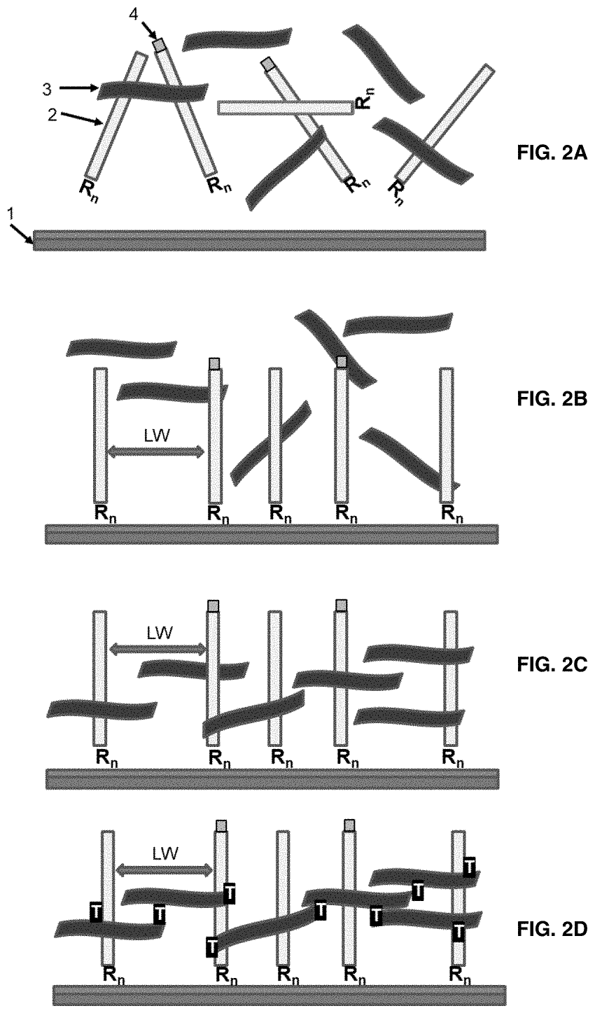

The covalent attachment of any one of these nucleic acid probes does not occur to the underlying surface directly, but is instead mediated through a relatively long, bi-functional polymeric linker that is capable of both chemical reaction with the surface and also capable of efficient UV-initiated crosslinking with the nucleic acid probe. The mechanics of this process is spontaneous 3D self assembly and is illustrated in FIG. 1A-FIG. 1D. As seen in FIG. 1A, the components required to fabricate this microarray system are:

(a) an unmodified nucleic acid probe 3 such as an oligonucleotide, PCR or isothermal amplicon, plasmid or genomic DNA;

(b) a chemically activatable surface 1 with chemically activatable groups (designated “X”) compatible for reacting with a primary amine such as. epoxysilane, isocyanate, succinimide, carbodiimide, aldehyde.

(c) bifunctional polymer linkers 2 such as a natural or modified OligodT, amino polysaccharide, amino polypeptide suitable for coupling to chemically activatable groups on the support surface, each attached with a fluorescent label 4; and

(d) a solvent comprising water and a high boiling point, water-miscible liquid such as glycerol, DMSO or propanediol (water to solvent ratio between 10:1 and 100:1).

Table 1 shows examples of chemically activatable groups and matched reactive groups on the bifunctional polymer linker for mere illustration purposes only and does not in any way preclude use of other combinations of matched reactive pairs.

TABLE 1
Covalent Attachment of Bifunctional Polymeric
Linker to an Activated Surfaces

| | Matched Reactive | | |
|---|---|---|---|
| | Group on | Specific Implementation | |
| Activated Surface | Bifunctional | as Bifunctional polymeric | |
| Moiety | Linker | linker | |
| Epoxysilane | Primary Amine | (1) | Amine-modified OligodT |
| | | | (20-60 bases) |
| | | (2) | Chitosan (20-60 subunits) |
| | | (3) | Lysine containing |
| | | | polypeptide (20-60aa) |
| EDC Activated | Primary Amine | (4) | Amine-modified OligodT |
| Carboxylic Acid | | | (20-60 bases) |
| | | (5) | Chitosan (20-60 subunits) |
| | | (6) | Lysine containing |
| | | | polypeptide (20-60aa) |
| N-hydroxy- | Primary Amine | (7) | Amine-modified OligodT |
| succinimide | | | (20-60 bases) |
| (NHS) | | (8) | Chitosan (20-60 subunits) |
| | | (9) | Lysine containing |
| | | | polypeptide (20-60aa) |

When used in the present invention, the chemically activatable surface, bifunctional polymer linkers and unmodified nucleic acid probes are included as a solution to be applied to a chemically activated surface 4 by ordinary methods of fabrication used to generate DNA Hybridization tests such as contact printing, piezo electric printing, ink jet printing, or pipetting.

Microarray fabrication begins with application of a mixture of the chemically activatable surface, bifunctional polymer linkers and unmodified nucleic acid probes to the surface. The first step is reaction and covalent attachment of the bifunctional linker to the activated surface (FIG. 1B). In general, the chemical concentration of the bi-functional linker is set to be such that less than 100% of the reactive sites on the surface form a covalent linkage to the bi-functional linker. At such low density, the average distance between bi-functional linker molecules defines a spacing denoted lattice width (“LW” in FIG. 1B).

In the second step, the water in the solvent is evaporated to concentrate the DNA and bifunctional linker via evaporation of water from the solvent (FIG. 1C). Generally, use of pure water as the solvent during matrix fabrication is disadvantageous because water is very quickly removed by evaporation due to a high surface area/volume ratio. To overcome this, in the present invention, a mixture of water with a high boiling point water-miscible solvent such as glycerin, DMSO or propanediol was used as solvent. In this case, upon evaporation, the water component will evaporate but not the high boiling point solvent. As a result, molecular reactants—DNA and bifunctional linker are progressively concentrated as the water is lost to evaporation. In the present invention, the ratio or water to high boiling point solvent is kept between 10:1 and 100:1. Thus, in the two extreme cases, upon equilibrium, volume of the fluid phase will reduce due to water evaporation to between 1/100th and 1/10^{th} the original volume, thus giving rise to a 100-fold to 10-fold increase in reactant concentration. Such controlled evaporation is crucial to the present invention since it controls the vertical spacing (Vertical Separation, “VG” in FIG. 1C) between nucleic acid probes, which is inversely related to the extent of evaporative concentration.

In the third step, the terminal Thymidine bases in the nucleic acid probes are UV crosslinked to the bifunctional linker within the evaporated surface (FIG. 1D). This process is mediated by the well-known photochemical reactivity of the Thymidine base that leads to the formation of covalent linkages to other thymidine bases in DNA or photochemical reaction with proteins and carbohydrates. If the bifunctional crosslinker is OligodT, then the crosslinking reaction will be bi-directional, that is, the photochemistry can be initiated in either the nucleic acid probe or the bifunctional OligodT linker. On the other hand, if the bifunctional linker is an amino polysaccharide such as chitosan or a polyamino acid, with a lysine or histidine in it, then the photochemistry will initiate in the nucleic acid probe, with the bifunctional linker being the target of the photochemistry.

Microarray System with Unmodified Solid Support for Non-Covalent Attachment

In this microarray system, attachment of the nucleic acid probes does not occur to the underlying surface directly, but is instead mediated through a relatively long, bi-functional polymeric linker that binds non-covalently with the solid support, but covalently with the nucleic acid probes via UV-initiated crosslinking. The mechanics of this process is spontaneous 3D self assembly and is illustrated in FIGS. 2A-2D. As seen in FIG. 2A, the components required to fabricate this microarray system are:

(1) an unmodified nucleic acid probe 3 such as an oligonucleotide, PCR or isothermal amplicon, plasmid or genomic DNA;

(2) an unmodified solid support 1;

(3) bifunctional polymer linkers 2 such as OligodT or a amino polysaccharide, amino polypeptide, that inherently have or are modified to have functional groups (designated “R”) compatible for adsorptive binding to the solid support, each having a fluorescent label 4; and

(4) a solvent comprising water and a high boiling point, water-miscible liquid such as glycerol, DMSO or propanediol (water to solvent ratio between 10:1 and 100:1);

Table 2 shows examples of unmodified support surfaces and matched absorptive groups on the bifunctional polymer linker for mere illustration purposes only and does not in any way precludes the use of other combinations of these.

TABLE 2
Non-Covalent Attachment of Bi-Functional
Polymeric Linker to an Inert Surface

| Representative | Matched Adsorptive | |
|---|---|---|
| support | Group on Bifunctional | Specific Bifunctional |
| surface | Linker (Rₙ) | polymeric linker |
| glass | Single Stranded Nucleic | OligodT (30-60 bases) |
| | Acid > 10 bases | |
| glass | Amine-Polysaccharide | Chitosan (30-60 subunits) |
| glass | Extended Planar | OligodT (30-60 bases)-5′- |
| | Hydrophobic Groups, | Digoxigenin |
| | e.g. Digoxigenin | |
| polycarbonate | Single Stranded Nucleic | Oligo-dT (30-60 bases) |
| | Acid > 10 bases | |
| polycarbonate | Amine-Polysaccharide | Chitosan (30-60 subunits) |
| polycarbonate | Extended Planar | OligodT (30-60 bases)-5′- |
| | Hydrophobic Groups, | Digoxigenin |
| | e.g. Digoxigenin | |
| graphene | Extended Planar | OligodT (30-60 bases)-5′ |
| | Hydrophobic Groups, | pyrene |
| | e.g. pyrene | |
| graphene | Extended Planar | OligodT (30-60 bases)-5′- |
| | Hydrophobic Groups, | CY-5 dye |
| | e.g. CY-5 dye | |
| graphene | Extended Planar | OligodT (30-60 bases)-5′- |
| | Hydrophobic Groups, | Digoxigenin |
| | e.g. Digoxigenin | |
| gold | Extended Planar | OligodT (30-60 bases)-5′ |
| | Hydrophobic Groups, | pyrene |
| | e.g. pyrene | |
| gold | Extended Planar | OligodT (30-60 bases)-5′- |
| | Hydrophobic Groups, | CY-5 dye |
| | e.g. CY-5 dye | |
| gold | Extended Planar | OligodT (30-60 bases)-5′ |
| | Hydrophobic Groups, | Digoxigenin |
| | e.g. Digoxigenin | |

When used in the present invention, components 1-3 are included as a solution to be applied to the solid support surface by ordinary methods of fabrication used to generate DNA Hybridization tests such as contact printing, piezo electric printing, ink jet printing, or pipetting.

Microarray fabrication begins with application of a mixture of the components (1)-(3) to the surface. The first step is adsorption of the bifunctional linker to the support surface (FIG. 2B). The concentration of the bi-functional linker is set so the average distance between bi-functional linker molecules defines a spacing denoted as lattice width (“LW” in FIG. 2B).

In the second step, the water in the solvent is evaporated to concentrate the DNA and bifunctional linker via evaporation of water from the solvent (FIG. 2C). Generally, use of pure water as the solvent during matrix fabrication is disadvantageous because water is very quickly removed by evaporation due to a high surface area/volume ratio. To overcome this, in the present invention, a mixture of water with a high boiling point water-miscible solvent such as glycerin, DMSO or propanediol was used as solvent. In this case, upon evaporation, the water component will evaporate but not the high boiling point solvent. As a result, molecular reactants—DNA and bifunctional linker are progressively concentrated as the water is lost to evaporation. In the present invention, the ratio or water to high boiling point solvent is kept between 10:1 and 100:1. Thus, in the two extreme cases, upon equilibrium, volume of the fluid phase will reduce due to water evaporation to between 1/100th and 1/10^{th} the original volume, thus giving rise to a 100-fold to 10-fold increase in reactant concentration.

In the third step, the terminal Thymidine bases in the nucleic acid probes are UV crosslinked to the bifunctional linker within the evaporated surface (FIG. 2D). This process is mediated by the well-known photochemical reactivity of the Thymidine base that leads to the formation of covalent linkages to other thymidine bases in DNA or photochemical reaction with proteins and carbohydrates. If the bifunctional crosslinker is OligodT, then the crosslinking reaction will be bi-directional, that is, the photochemistry can be initiated in either the nucleic acid probe or the bifunctional OligodT linker. On the other hand, if the bifunctional linker is an amino polysaccharide such as chitosan or a polyamino acid, with a lysine or histidine in it, then the photochemistry will initiate in the nucleic acid probe, with the bifunctional linker being the target of the photochemistry.

Although such non-covalent adsorption described in the first step is generally weak and reversible, when occurring in isolation, in the present invention it is taught that if many such weak adsorptive events between the bifunctional polymeric linker and the underlying surface occur in close proximity, and if the closely packed polymeric linkers are subsequently linked to each other via Thymidine-mediated photochemical crosslinking, the newly created extended, multi-molecular (crosslinked) complex will be additionally stabilized on the surface, thus creating a stable complex with the surface in the absence of direct covalent bonding to that surface.

The present invention works very efficiently for the linkage of synthetic oligonucleotides as nucleic acid probes to form a microarray-based hybridization device for the analysis of microbial DNA targets. However, it is clear that the same invention may be used to link PCR amplicons, synthetic oligonucleotides, isothermal amplification products, plasmid DNA or genomic DNA fragment as nucleic acid probes. It is also clear that the same technology could be used to manufacture hybridization devices that are not microarrays.

DNA nucleic acid probes were formulated as described in Table 3, to be deployed as described above and illustrated in FIG. 1 or 2. A set of 48 such probes (Table 4) were designed to be specific for various sequence determinants of microbial DNA and each was fabricated so as to present a string of 5-7 T bases at each end, to facilitate their UV-crosslinking to form a covalently linked microarray element, as described above and illustrated in FIG. 1. Each of the 48 different probes was printed in triplicate to form a 144 element (12×12) microarray having sequences shown in Table 3.

TABLE 3
Representative Conditions of use of the Present Invention

| | Unique sequence | |
|---|---|---|
| | Oligonucleotide | 5′ labelled OligodT |
| Nucleic acid | 30-38 bases Long | Fluorescent marker 30 |
| probe Type | 7 T's at each end | bases Long(marker) |
| Nucleic acid | 50 mM | 0.15 mM |
| probe Concentration | | |
| Bifunctional Linker | OligodT 30 bases long | |
| | Primary amine at 3′ | |
| | terminus | |
| Bifunctional Linker | 1 mM | |
| Concentration | | |
| High Boiling | Water:Propanediol, | |
| point Solvent | 100:1 | |
| Surface | Epoxysilane on | |
| | borosilicate glass | |
| UV Crosslinking | 300 millijoule | |
| Dose (mjoule) | | |

TABLE 4
Nucleic acid probes Linked to the Microarray
Surface via the Present Invention

| | | |
|---|---|---|
| SEQ ID NO: 132 | Negative control | TTTTTTCTACTACCTATGCTGATTCACTCTTTT |
| | | T |
| SEQ ID NO: 129 | Imager Calibration | TTTTCTATGTATCGATGTTGAGAAATTTTTTT |
| | (High) | |
| SEQ ID NO: 130 | Imager Calibration | TTTTCTAGATACTTGTGTAAGTGAATTTTTTT |
| | (Low) | |
| SEQ ID NO: 131 | Imager Calibration | TTTTCTAAGTCATGTTGTTGAAGAATTTTTTT |
| | (Medium) | |
| SEQ ID NO: 126 | Cannabis ITS1 DNA | TTTTTTAATCTGCGCCAAGGAACAATATTTTT |
| | Control 1 | TT |
| SEQ ID NO: 127 | Cannabis ITS1 DNA | TTTTTGCAATCTGCGCCAAGGAACAATATTTT |
| | Control 2 | TT |
| SEQ ID NO: 128 | Cannabis ITS1 DNA | TTTATTTCTTGCGCCAAGGAACAATATTTTAT |
| | Control 3 | TT |
| SEQ ID NO: 86 | Total Yeast and | TTTTTTTTGAATCATCGARTCTTTGAACGCAT |
| | Mold (High | TTTTTT |
| | sensitivity) | |
| SEQ ID NO: 87 | Total Yeast and | TTTTTTTTGAATCATCGARTCTCCTTTTTTT |
| | Mold (Low | |
| | sensitivity) | |
| SEQ ID NO: 88 | Total Yeast and | TTTTTTTTGAATCATCGARTCTTTGAACGTTTT |
| | Mold (Medium | TTT |
| | sensitivity) | |
| SEQ ID NO: 132 | Negative control | TTTTTTCTACTACCTATGCTGATTCACTCTTTT |
| | | T |
| SEQ ID NO: 92 | Aspergillus | TTTCTTTTCGACACCCAACTTTATTTCCTTATT |
| | fumigatus 1 | T |
| SEQ ID NO: 90 | Aspergillus flavus 1 | TTTTTTCGCAAATCAATCTTTTTCCAGTCTTTT |
| | | T |
| SEQ ID NO: 95 | Aspergillus niger 1 | TTTTTTCGACGTTTTCCAACCATTTCTTTT |
| SEQ ID NO: 100 | Botrytis spp. | TTTTTTTCATCTCTCGTTACAGGTTCTCGGTT |
| | | CTTTTTTT |
| SEQ ID NO: 108 | Fusarium spp. | TTTTTTTTAACACCTCGCRACTGGAGATTTTT |
| | | TT |
| SEQ ID NO: 89 | Alternaria spp | TTTTTTCAAAGGTCTAGCATCCATTAAGTTTTT |
| | | T |
| SEQ ID NO: 123 | Rhodoturula spp. | TTTTTTCTCGTTCGTAATGCATTAGCACTTTTT |
| | | T |
| SEQ ID NO: 117 | Penicillium paxilli | TTTTTTCCCCTCAATCTTTAACCAGGCCTTTTT |
| | | T |
| SEQ ID NO: 116 | Penicillium oxalicum | TTTTTTACACCATCAATCTTAACCAGGCCTTT |
| | | TT |
| SEQ ID NO: 118 | Penicillium spp. | TTTTTTCAACCCAAATTTTTATCCAGGCCTTTT |
| | | T |
| SEQ ID NO: 102 | Candida spp. | TTTTTTTGTTTGGTGTTGAGCRATACGTATTTT |
| | Group 1 | T |
| SEQ ID NO: 103 | Candida spp. | TTTTACTGTTTGGTAATGAGTGATACTCTCAT |
| | Group 2 | TTT |
| SEQ ID NO: 124 | Stachybotrys spp | TTTCTTCTGCATCGGAGCTCAGCGCGTTTTAT |
| | | TT |
| SEQ ID NO: 125 | Trichoderma spp. | TTTTTCCTCCTGCGCAGTAGTTTGCACATCTT |
| | | TT |
| SEQ ID NO: 105 | Cladosporium spp. | TTTTTTTTGTGGAAACTATTCGCTAAAGTTTTT |
| | | T |
| SEQ ID NO: 121 | Podosphaera spp. | TTTTTTTTAGTCAYGTATCTCGCGACAGTTTTT |
| | | T |
| SEQ ID NO: 132 | Negative control | TTTTTTCTACTACCTATGCTGATTCACTCTTTT |
| | | T |
| SEQ ID NO: 37 | Total Aerobic | TTTTTTTTTCCTACGGGAGGCAGTTTTTTT |
| | bacteria (High) | |
| SEQ ID NO: 38 | Total Aerobic | TTTTTTTTCCCTACGGGAGGCATTTTTTTT |
| | bacteria (Medium) | |
| SEQ ID NO: 39 | Total Aerobic | TTTATTTTCCCTACGGGAGGCTTTTATTTT |
| | bacteria (Low) | |
| SEQ ID NO: 47 | Bile-tolerant Gram- | TTTTTCTATGCAGTCATGCTGTGTGTRTGTCT |
| | negative (High) | TTTT |
| SEQ ID NO: 48 | Bile-tolerant Gram- | TTTTTCTATGCAGCCATGCTGTGTGTRTTTTT |
| | negative (Medium) | TT |
| SEQ ID NO: 49 | Bile-tolerant Gram- | TTTTTCTATGCAGTCATGCTGCGTGTRTTTTT |
| | negative (Low) | TT |
| SEQ ID NO: 53 | Coliform/ | TTTTTTCTATTGACGTTACCCGCTTTTTTT |
| | Enterobacteriaceae | |
| SEQ ID NO: 81 | stx1 gene | TTTTTTCTTTCCAGGTACAACAGCTTTTTT |
| SEQ ID NO: 82 | stx2 gene | TTTTTTGCACTGTCTGAAACTGCCTTTTTT |
| SEQ ID NO: 59 | etuf gene | TTTTTTCCATCAAAGTTGGTGAAGAATCTTTT |
| | | TT |
| SEQ ID NO: 132 | Negative control | TTTTTTCTACTACCTATGCTGATTCACTCTTTT |
| | | T |
| SEQ ID NO: 65 | Listeria spp. | TTTTCTAAGTACTGTTGTTAGAGAATTTTT |
| SEQ ID NO: 56 | Aeromonas spp. | TTATTTTCTGTGACGTTACTCGCTTTTATT |
| SEQ ID NO: 78 | Staphylococcus | TTTATTTTCATATGTGTAAGTAACTGTTTTATT |
| | aureus 1 | T |
| SEQ ID NO: 49 | Campylobacter spp. | TTTTTTATGACACTTTTCGGAGCTCTTTTT |
| SEQ ID NO: 72 | Pseudomonas | TTTATTTTAAGCACTTTAAGTTGGGATTTTATT |
| | spp. 3 | T |
| SEQ ID NO: 53 | Clostridium spp. | TTTTCTGGAMGATAATGACGGTACAGTTTT |
| SEQ ID NO: 42 | Escherichia coli/ | TTTTCTAATACCTTTGCTCATTGACTCTTT |
| | Shigella 1 | |
| SEQ ID NO: 74 | Salmonella enterica/ | TTTTTTTGTTGTGGTTAATAACCGATTTTT |
| | Enterobacter 1 | |
| SEQ ID NO: 61 | invA gene | TTTTTTTATTGATGCCGATTTGAAGGCCTTTTT |
| | | T |

The set of 48 different probes of Table 4 were formulated as described in Table 3, then printed onto epoxysilane coated borosilicate glass, using an Gentics Q-Array mini contact printer with Arrayit SMP pins, which deposit about 1 nL of formulation per spot. As described in FIG. 1, the arrays thus printed were then allowed to react with the epoxisilane surface at room temperature, and then evaporate to remove free water, also at room temperature. Upon completion of the evaporation step (typically overnight) the air-dried microarrays were then UV treated in a Statolinker UV irradiation system: 300 mjoules of irradiation at 254 nm to initiate thymidine-mediated crosslinking. The microarrays are then ready for use, with no additional need for washing or capping.

### Example 2

Using the 3-dimensional lattice microarray system for DNA analysis

### Sample Processing

Harvesting Pathogens from plant surface comprises the following steps:

1) Wash the plant sample or tape pull in 1× phosphate buffered saline (PBS);

2) Remove plant material/tape;

3) Centrifuge to pellet cells & discard supernatant;

4) Resuspend in PathogenDx (PathogenDX, Inc.) Sample Prep Buffer pre-mixed with Sample Digestion Buffer;

5) Heat at 55° C. for 45 minutes;

6) Vortex to dissipate the pellet;

7) Heat at 95° C. for 15 minutes; and

8) Vortex and centrifuge briefly before use in PCR.

### Amplification by PCR

The sample used for amplification and hybridization analysis was a Cannabis flower wash from a licensed Cannabis lab. The washed flower material was then pelleted by centrifugation. The pellet was then digested with proteinaseK, then spiked with a known amount of Salmonella DNA before PCR amplification.

The Salmonella DNA spiked sample was then amplified with PCR primers (P1-Table 5) specific for the 16S region of Enterobacteriaceae in a tandem PCR reaction to first isolate the targeted region (PCR Reaction #1) and also PCR primers (P1-Table 5) which amplify a segment of Cannabis DNA (ITS) used as a positive control.

The product of PCR Reaction #1 (1 μL) was then subjected to a second PCR reaction (PCR Reaction #2) which additionally amplified and labelled the two targeted regions (16S, ITS) with green CY3 fluorophore labeled primers (P2-Table 5). The product of the PCR Reaction #2 (50 μL) was then diluted 1-1 with hybridization buffer (4×SSC+5×Denhardt's solution) and then applied directly to the microarray for hybridization.

TABLE 5
PCR Primers and PCR conditions used in amplification
PCR primers (P1) for PCR Reaction #1
Cannabis ITS1 1 ° FP*- TTTGCAACAGCAGAACGACCCGTGA
Cannabis ITS1 1 ° RP*- TTTCGATAAACACGCATCTCGATTG
Enterobacteriaceae 16S 1 ° FP- TTACCTTCGGGCCTCTTGCCATCRGATGTG
Enterobacteriaceae 16S 1 RP- TTGGAATTCTACCCCCCTCTACRAGACTCAAGC
PCR primers (P2) for PCR Reaction #2
Cannabis ITS1 2 ° FP- TTTCGTGAACACGTTTTAAACAGCTTG
Cannabis ITS1 2 ° RP- (Cy3)TTTTCCACCGCACGAGCCACGCGAT
Enterobacteriaceae 16S 2 ° FP- TTATATTGCACAATGGGCGCAAGCCTGATG
Enterobacteriaceae 16S 2 °°RP-(Cy3)TTTTGTATTACCGCGGCTGCTGGCA

| PCR Reagent | Primary PCR Concentration | Secondary PCR Concentration |
|---|---|---|
| PCR Buffer | 1X | 1X |
| MgCl₂ | 2.5 mM | 2.5 mM |
| BSA | 0.16 mg/mL | 0.16 mg/mL |
| dNTP's | 200 mM | 200 mM |
| Primer mix | 200 nM each | 50 nM - FP/200 nM RP |
| Taq Polymerase | 1.5 Units | 1.5 Units |

Program for PCR Reaction #1

| | | | | |
|---|---|---|---|---|
| 95 ° C., 4 min | 98 ° C., 30s | 61 ° C., 30s | 72 ° C., 60s | 72 ° C., 7 min |

25X

Program for PCR Reaction #2

| | | | | |
|---|---|---|---|---|
| 95 ° C., 4 min | 98 ° C., 20s | 61 ° C., 20s | 72 ° C., 30s | 72 ° C., 7 min |

25X

*FP, Forward Primer;
*RP, Reverse Primer

### Hybridization

Because the prior art method of microarray manufacture allows DNA to be analyzed via hybridization without the need for pre-treatment of the microarray surface, the use of the microarray is simple, and involves 6 manual or automated pipetting steps.

1) Pipette the amplified DNA+binding buffer onto the microarray

2) Incubate for 30 minutes to allow DNA binding to the microarray (typically at room temperature, RT)

3) Remove the DNA+binding buffer by pipetting

4) Pipette 50 uL of wash buffer onto the microarray (0.4×SSC+0.5×Denhardt's) and incubate 5 min at RT.

5) Remove the wash buffer by pipetting

6) Repeat steps 4 and 5

7) Perform image analysis at 532 nm and 635 nm to detect the probe spot location (532 nm) and PCR product hybridization (635 nm).

### Image Analysis

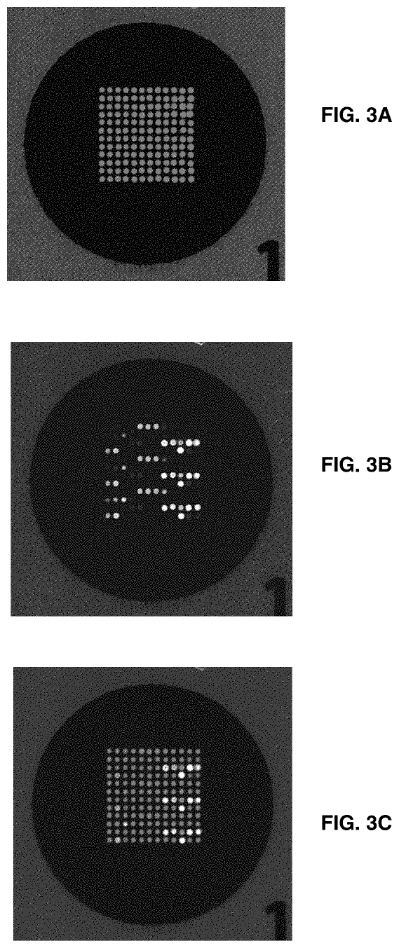

Image Analysis was performed at two wavelengths (532 nm and 635 nm) on a raster-based confocal scanner: GenePix 4000B Microarray Scanner, with the following imaging conditions: 33% Laser power, 400PMT setting at 532 nm/33% Laser Power, 700PMT setting at 635 nm. FIG. 3 shows an example of the structure and hybridization performance of the microarray.

FIG. 3A reveals imaging of the representative microarray, described above, after hybridization and washing, as visualized at 635 nm. The 635 nm image is derived from signals from the (red) CY5 fluor attached to the 5′ terminus of the bifunctional polymer linker OligodT which had been introduced during microarray fabrication as a positional marker in each microarray spot (see FIG. 1 and Table 3). The data in FIG. 3A confirm that the Cy5-labelled OligodT has been permanently linked to the microarray surface, via the combined activity of the bi-functional linker and subsequent UV-crosslinking, as described in FIG. 1.

FIG. 3B reveals imaging of the representative microarray described above after hybridization and washing as visualized at 532 nm. The 532 nm image is derived from signals from the (green) CY3 fluor attached to the 5′ terminus of PCR amplified DNA obtained during PCR Reaction #2. It is clear from FIG. 3B that only a small subset of the 48 discrete probes bind to the Cy3-labelled PCR product, thus confirming that the present method of linking nucleic acid probes to form a microarray (FIG. 1) yields a microarray product capable of sequence specific binding to a (cognate) solution state target. The data in FIG. 3B reveal the underlying 3-fold repeat of the data (i.e., the array is the same set of 48 probes printed three times as 3 distinct sub-arrays to form the final 48×3=144 element microarray. The observation that the same set of 48 probes can be printed 3-times, as three repeated sub-domains show that the present invention generates microarray product that is reproducible.

FIG. 3C reveals imaging of the representative microarray, described above, after hybridization and washing, as visualized with both the 532 nm and 635 nm images superimposed. The superimposed images display the utility of parallel attachment of a Cy5-labelled OligodT positional marker relative to the sequence specific binding of the CY3-labelled PCR product.

### Example 3

### Position of Pathogen Specific PCR Primers

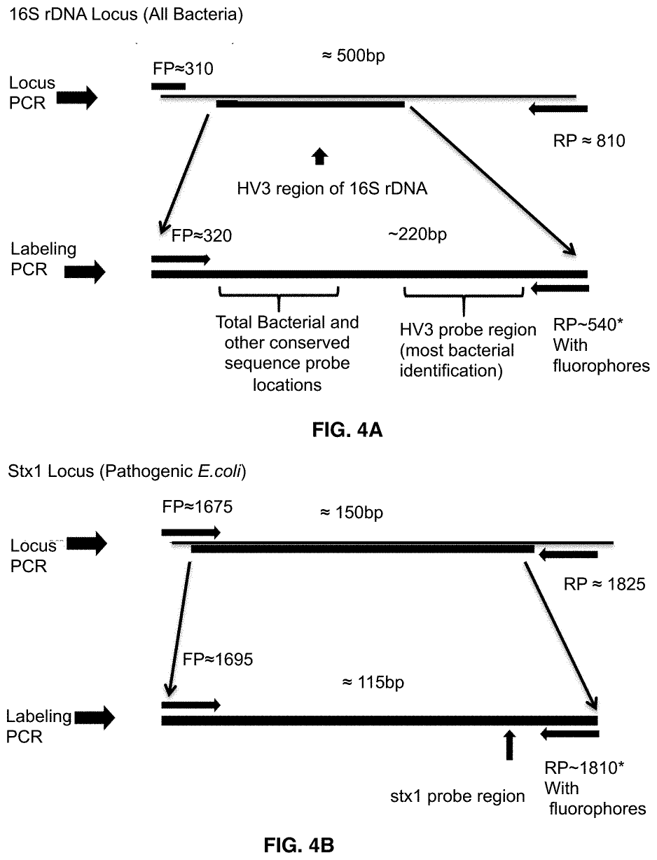

FIG. 4A shows an exemplar of the first PCR step. As is standard, such PCR reactions are initiated by the administration of PCR Primers. Primers define the start and stopping point of the PCR based DNA amplification reaction. In this embodiment, a pair of

PCR reactions is utilized to support the needed DNA amplification. In general, such PCR amplification is performed in series: a first pair of PCRs, with the suffix “P1” in FIG. 4A are used to amplify about 1 μL of any unpurified DNA sample, such as a raw Cannabis leaf wash for example. About 1 μL of the product of that first PCR reaction is used as the substrate for a second PCR reaction that is used to affix a fluorescent dye label to the DNA, so that the label may be used to detect the PCR product when it binds by hybridization to the microarray. The primer sequences for the first and second PCRs are shown in Table 6. The role of this two-step reaction is to avert the need to purify the pathogen DNA to be analyzed. The first PCR reaction, with primers “P1” is optimized to accommodate the raw starting material, while the second PCR primer pairs “P2” are optimized to obtain maximal DNA yield, plus dye labeling from the product of the first reaction. Taken in the aggregate, the sum of the two reactions obviates the need to either purify or characterize the pathogen DNA of interest.

FIG. 4A reveals at low resolution the 16S rDNA region which is amplified in an embodiment, to isolate and amplify a region which may be subsequently interrogated by hybridization. The DNA sequence of this 16S rDNA region is known to vary greatly among different bacterial species. Consequently, having amplified this region by two step PCR, that sequence variation may be interrogated by the subsequent microarray hybridization step.

FIG. 4B displays the stx1 gene locus which is present in the most important pathogenic strains of E coli and which encodes Shigatoxin 1. Employing the same two-step PCR approach, a set of two PCR primer pairs were designed which, in tandem, can be used to amplify and label unprocessed bacterial samples to present the stx1 locus for analysis by microarray-based DNA hybridization.

TABLE 6
First and Second PCR Primers

| | | |
|---|---|---|
| SEQ ID NO. | Primer target | Primer sequence |

First PCR Primers (P1) for the first amplification step

| | | |
|---|---|---|
| SEQ ID NO: 1 | 16S rDNA HV3 Locus | TTTCACAYTGGRACTGAGACACG |
| | (Bacteria) | |
| SEQ ID NO: 2 | 16S rDNA HV3 Locus | TTTGACTACCAGGGTATCTAATCCTG |
| | (Bacteria) | T |
| SEQ ID NO: 3 | Stx1 Locus | TTTATAATCTACGGCTTATTGTTGAA |
| | (Pathogenic E. coli) | CG |
| SEQ ID NO: 4 | Stx1 Locus | TTTGGTATAGCTACTGTCACCAGACA |
| | (Pathogenic E. coli) | ATG |
| SEQ ID NO: 5 | Stx2 Locus | TTTGATGCATCCAGAGCAGTTCTGC |
| | (Pathogenic E. coli) | G |
| SEQ ID NO: 6 | Stx2 Locus | TTTGTGAGGTCCACGTCTCCCGGCG |
| | (Pathogenic E. coli) | TC |
| SEQ ID NO: 7 | InvA Locus (Salmonella) | TTTATTATCGCCACGTTCGGGCAATT |
| | | CG |
| SEQ ID NO: 8 | InvA Locus (Salmonella) | TTTCTTCATCGCACCGTCAAAGGAAC |
| | | CG |
| SEQ ID NO: 9 | tuf Locus (All E. coli) | TTTCAGAGTGGGAAGCGAAAATCCT |
| | | G |
| SEQ ID NO: 10 | tuf Locus (All E. coli) | TTTACGCCAGTACAGGTAGACTTCTG |
| SEQ ID NO: 11 | 16S rDNA | TTACCTTCGGGCCTCTTGCCATCRG |
| | Enterobacteriaceae HV3 | ATGTG |
| | Locus | |
| SEQ ID NO: 12 | 16S rDNA | TTGGAATTCTACCCCCCTCTACRAGA |
| | Enterobacteriaceae HV3 | CTCAAGC |
| | Locus | |
| SEQ ID NO: 13 | ITS2 Locus | TTTACTTTYAACAAYGGATCTCTTGG |
| | (All Yeast, Mold/Fungus) | |
| SEQ ID NO: 14 | ITS2 Locus | TTTCTTTTCCTCCGCTTATTGATATG |
| | (All Yeast, Mold/Fungus) | |
| SEQ ID NO: 15 | ITS2 Locus | TTTAAAGGCAGCGGCGGCACCGCGT |
| | (Aspergillus species) | CCG |
| SEQ ID NO: 16 | ITS2 Locus | TTTTCTTTTCCTCCGCTTATTGATATG |
| | (Aspergillus species) | |
| SEQ ID NO: 17 | ITS1 Locus | TTTGCAACAGCAGAACGACCCGTGA |
| | (Cannabis/Plant) | |
| SEQ ID NO: 18 | ITS1 Locus | TTTCGATAAACACGCATCTCGATTG |
| | (Cannabis/Plant) | |

Second PCR Primers (P2) for the second labeling amplification step

| | | |
|---|---|---|
| SEQ ID NO: 19 | 16S rDNA HV3 Locus | TTTACTGAGACACGGYCCARACTC |
| | (All Bacteria) | |
| SEQ ID NO: 20 | 16S rDNA HV3 Locus | TTTGTATTACCGCGGCTGCTGGCA |
| | (All Bacteria) | |
| SEQ ID NO: 21 | Stx1 Locus | TTTATGTGACAGGATTTGTTAACAGG |
| | (Pathogenic E. coli) | AC |
| SEQ ID NO: 22 | Stx1 Locus | TTTCTGTCACCAGACAATGTAACCGC |
| | (Pathogenic E. coli) | TG |
| SEQ ID NO: 23 | Stx2 Locus | TTTTGTCACTGTCACAGCAGAAG |
| | (Pathogenic E. coli) | |
| SEQ ID NO: 24 | Stx2 Locus | TTTGCGTCATCGTATACACAGGAGC |
| | (Pathogenic E. coli) | |
| SEQ ID NO: 25 | InvA Locus | TTTTATCGTTATTACCAAAGGTTCAG |
| | (All Salmonella) | |
| SEQ ID NO: 26 | InvA Locus | TTTCCTTTCCAGTACGCTTCGCCGTT |
| | (All Salmonella) | CG |
| SEQ ID NO: 27 | tuf Locus (All E. coli) | TTTGTTGTTACCGGTCGTGTAGAAC |
| SEQ ID NO: 28 | tuf Locus (All E. coli) | TTTCTTCTGAGTCTCTTTGATACCAA |
| | | CG |
| SEQ ID NO: 29 | 16S rDNA | TTATATTGCACAATGGGCGCAAGCCT |
| | Enterobacteriaceae HV3 | GATG |
| | Locus | |
| SEQ ID NO: 30 | 16S rDNA | TTTTGTATTACCGCGGCTGCTGGCA |
| | Enterobacteriaceae HV3 | |
| | Locus | |
| SEQ ID NO: 31 | ITS2 Locus | TTTGCATCGATGAAGARCGYAGC |
| | (All Yeast, Mold/Fungus) | |
| SEQ ID NO: 32 | ITS2 Locus | TTTCCTCCGCTTATTGATATGC |
| | (All Yeast, Mold/Fungus) | |
| SEQ ID NO: 33 | ITS2 Locus | TTTCCTCGAGCGTATGGGGCTTTGT |
| | (Aspergillus species) | C |
| SEQ ID NO: 34 | ITS2 Locus | TITTTCCTCCGCTTATIGATATGC |
| | (Aspergillus species) | |
| SEQ ID NO: 133 | ITS2 Locus | TTTGCATCGATGAAGAACGCAGC |
| | (All Yeast, Mold/Fungus) | |
| SEQ ID NO: 134 | IT52 Locus (All Yeast, | TTTTCCTCCGCTTATTGATATGC |
| | Mold/Fungus) | |
| SEQ ID NO: 135 | Fungal RSG Primers | TTTACTTTCAACAAYGGATCTCTTG |
| | (All Fungus) | G |
| SEQ ID NO: 35 | ITS1 Locus | TTTCGTGAACACGTTTTAAACAGCTT |
| | (Cannabis/Plant) | G |
| SEQ ID NO: 36 | ITS1 Locus | TTTCCACCGCACGAGCCACGCGAT |
| | (Cannabis/Plant) | |

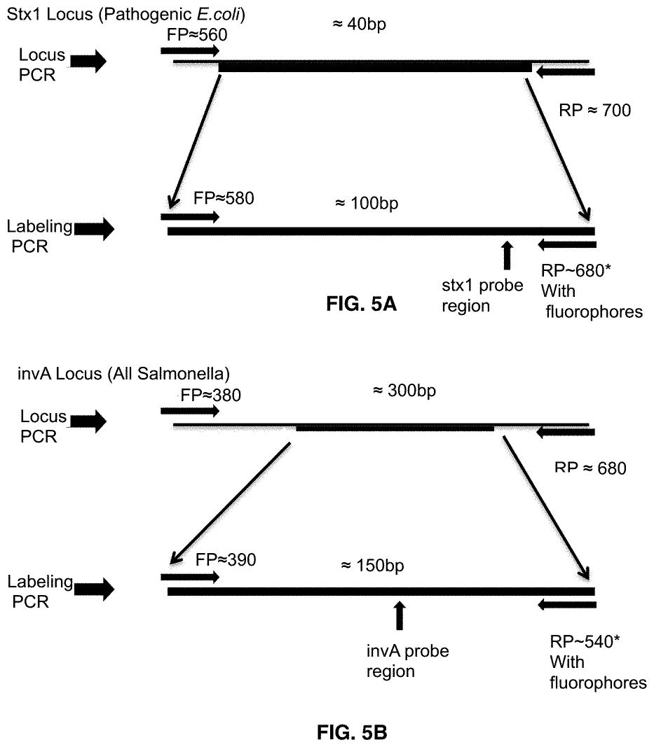

FIG. 5A displays the stx2 gene locus which is also present in the most important pathogenic strains of E coli and which encodes Shigatoxin 2. Employing the same two-step PCR approach, a set of two PCR primer pairs were designed which, in tandem, can be used to amplify and label unprocessed bacterial samples so as to present the stx2 locus for analysis by microarray-based DNA hybridization.

FIG. 5B displays the invA gene locus which is present in all strains of Salmonella and which encodes the InvAsion A gene product. Employing the same two-step PCR approach, a set of two PCR primer pairs were designed which, in tandem, can be used to amplify and label unprocessed bacterial samples so as to present the invA locus for analysis by microarray-based DNA hybridization.

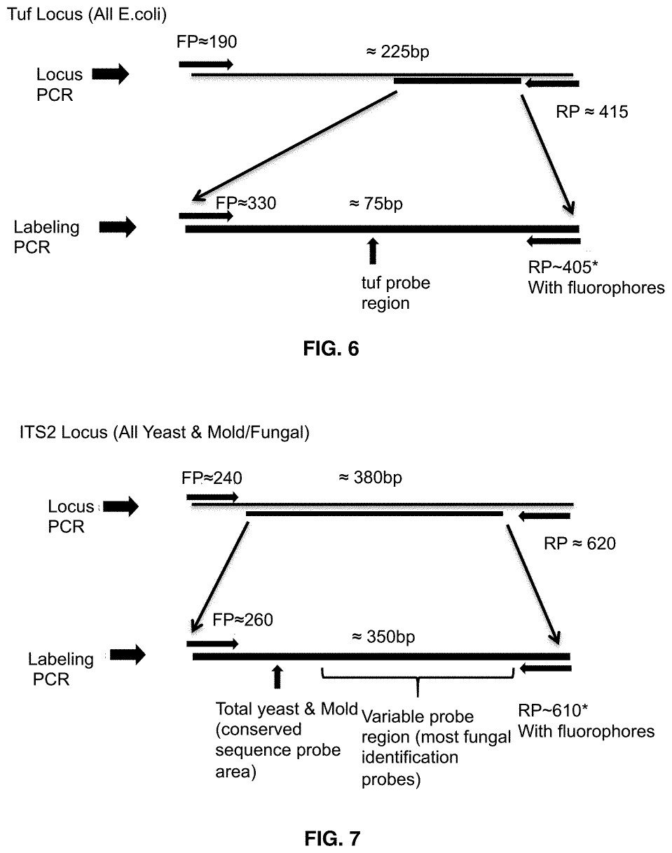

FIG. 6 displays the tuf gene locus which is present in all strains of E coli and which encodes the ribosomal elongation factor Tu. Employing the same two-step PCR approach, a set of two PCR primer pairs were designed which, in tandem, can be used to amplify and label unprocessed bacterial samples so as to present the tuf locus for analysis by microarray-based DNA hybridization.

FIG. 7 displays the ITS2 locus which is present in all eukaryotes, including all strains of yeast and mold and which encodes the intergenic region between ribosomal genes 5.8S and 28S. ITS2 is highly variable in sequence and that sequence variation can be used to resolve strain differences in yeast, and mold. Employing the same two-step PCR approach, a set of two PCR primer pairs were designed which, in tandem, can be used to amplify and label unprocessed yeast and mold samples so as to present the ITS2 locus for analysis by microarray-based DNA hybridization.

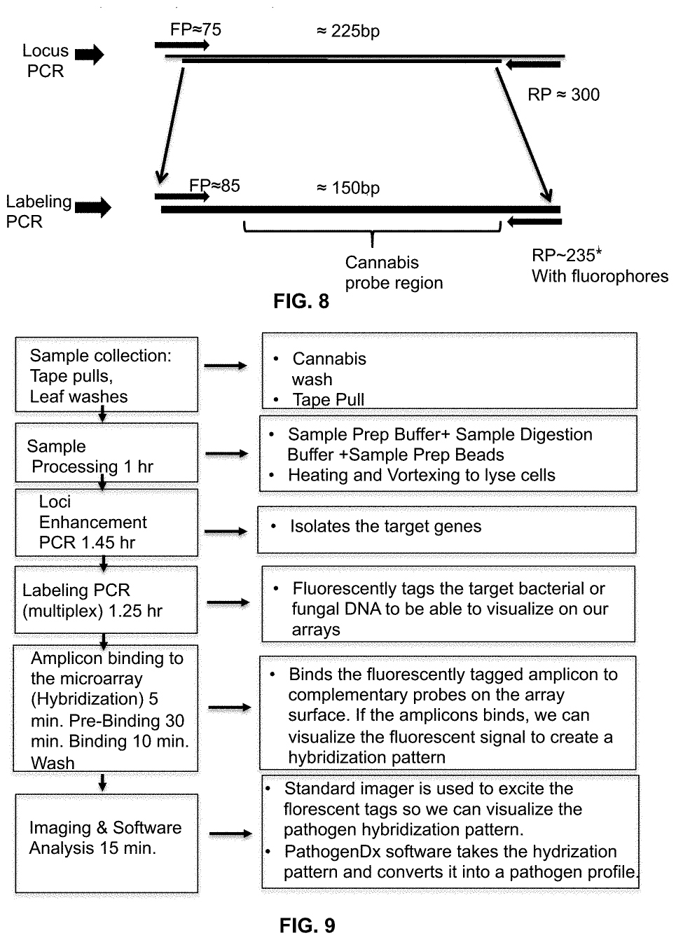

FIG. 8 displays the ITS1 gene locus which is present in all eukaryotes, including all plants and animals, which encodes the intergenic region between ribosomal genes 18S and 5.8S. ITS1 is highly variable in sequence among higher plants and that sequence variation can be used to identify plant species. Employing the same two-step PCR approach, a set of two PCR primer pairs were designed which, in tandem, can be used to amplify and label unprocessed Cannabis samples so as to present the ITS1 locus for analysis by microarray-based DNA hybridization. The identification and quantitation of the Cannabis sequence variant of ITS1 is used as an internal normalization standard in the analysis of pathogens recovered from the same Cannabis samples.

Table 7 displays representative oligonucleotide sequences which are used as microarray probes in an embodiment for DNA microarray-based analysis of bacterial 16S locus as described in FIG. 4. The sequence of those probes has been varied to accommodate the cognate sequence variation which occurs as a function of species difference among bacteria. In all cases, the probe sequences are terminated with a string of T's at each end, to enhance the efficiency of probe attachment to the microarray surface, at time of microarray manufacture. Table 8 shows sequences of the Calibration and Negative controls used in the microarray.

Table 9 displays representative oligonucleotide sequences which are used as microarray probes in an embodiment for DNA microarray-based analysis of eukaryotic pathogens (fungi, yeast & mold) based on their ITS2 locus as described in FIG. 7. Sequences shown in Table 8 are used as controls. The sequence of those probes has been varied to accommodate the cognate sequence variation which occurs as a function of species difference among fungi, yeast & mold. In all cases, the probe sequences are terminated with a string of T's at each end, to enhance the efficiency of probe attachment to the microarray surface, at time of microarray manufacture.

Table 10 displays representative oligonucleotide sequences which are used as microarray probes in an embodiment for DNA microarray-based analysis of Cannabis at the ITS1 locus (Cannabis spp.).

TABLE 7
Oligonucleotide probe sequence for the 16S Locus

| | | |
|---|---|---|
| SEQ ID NO: 37 | Total Aerobic bacteria (High) | TTTTTTTTTCCTACGGGAGGCAG |
| | | TTTTTTT |
| SEQ ID NO: 38 | Total Aerobic bacteria | TTTTTTTTCCCTACGGGAGGCATT |
| | (Medium) | TTTTTT |
| SEQ ID NO: 39 | Total Aerobic bacteria (Low) | TTTATTTTCCCTACGGGAGGCTTT |
| | | TATTTT |
| SEQ ID NO: 40 | Enterobacteriaceae (Low | TTTATTCTATTGACGTTACCCATT |
| | sensitivity) | TATTTT |
| SEQ ID NO: 41 | Enterobacteriaceae (Medium | TTTTTTCTATTGACGTTACCCGTT |
| | sensitivity) | TTTTTT |
| SEQ ID NO: 42 | Escherichia coli/Shigella 1 | TTTTCTAATACCTTTGCTCATTGA |
| | | CTCTTT |
| SEQ ID NO: 43 | Escherichia coli/Shigella 2 | TTTTTTAAGGGAGTAAAGTTAATA |
| | | TTTTTT |
| SEQ ID NO: 44 | Escherichia coli/Shigella 3 | TTTTCTCCTTTGCTCATTGACGTT |
| | | ATTTTT |
| SEQ ID NO: 45 | Bacillus spp. Group1 | TTTTTCAGTTGAATAAGCTGGCA |
| | | CTCTTTT |
| SEQ ID NO: 46 | Bacillus spp. Group2 | TTTTTTCAAGTACCGTTCGAATAG |
| | | TTTTTT |
| SEQ ID NO: 47 | Bile-tolerant Gram-negative | TTTTTCTATGCAGTCATGCTGTGT |
| | (High) | GTRTGTCTTTTT |
| SEQ ID NO: 48 | Bile-tolerant Gram-negative | TTTTTCTATGCAGCCATGCTGTGT |
| | (Medium) | GTRTTTTTTT |
| SEQ ID NO: 49 | Bile-tolerant Gram-negative | TTTTTCTATGCAGTCATGCTGCGT |
| | (Low) | GTRTTTTTTT |
| SEQ ID NO: 50 | Campylobacter spp. | TTTTTTATGACACTTTTCGGAGCT |
| | | CTTTTT |
| SEQ ID NO: 51 | Chromobacterium spp. | TTTTATTTTCCCGCTGGTTAATAC |
| | | CCTTTATTTT |
| SEQ ID NO: 52 | Citrobacter spp. Group1 | TTTTTTCCTTAGCCATTGACGTTA |
| | | TTTTTT |
| SEQ ID NO: 53 | Clostridium spp. | TTTTCTGGAMGATAATGACGGTA |
| | | CAGTTTT |
| SEQ ID NO: 54 | Coliform/Enterobacteriaceae | TTTTTTCTATTGACGTTACCCGCT |
| | | TTTTTT |
| SEQ ID NO: 55 | Aeromonas | TTTTTGCCTAATACGTRTCAACTG |
| | salmonicida/hydrophilia | CTTTTT |
| SEQ ID NO: 56 | Aeromonas spp. | TTATTTTCTGTGACGTTACTCGCT |
| | | TTTATT |
| SEQ ID NO: 57 | Alkanindiges spp. | TTTTTAGGCTACTGRTACTAATAT |
| | | CTTTTT |
| SEQ ID NO: 58 | Bacillus pumilus | TTTATTTAAGTGCRAGAGTAACTG |
| | | CTATTTTATT |
| SEQ ID NO: 59 | etuf gene | TTTTTTCCATCAAAGTTGGTGAAG |
| | | AATCTTTTTT |
| SEQ ID NO: 60 | Hafnia spp. | TTTTTTCTAACCGCAGTGATTGAT |
| | | CTTTTT |
| SEQ ID NO: 61 | invA gene | TTTTTTTATTGATGCCGATTTGAA |
| | | GGCCTTTTTT |
| SEQ ID NO: 62 | Klebsiella oxytoca | TTTTTTCTAACCTTATTCATTGAT |
| | | CTTTTT |
| SEQ ID NO: 63 | Klebsiella pneumoniae | TTTTTTCTAACCTTGGCGATTGAT |
| | | CTTTTT |
| SEQ ID NO: 64 | Legionella spp. | TTTATTCTGATAGGTTAAGAGCTG |
| | | ATCTTTATTT |
| SEQ ID NO: 65 | Listeria spp. | TTTTCTAAGTACTGTTGTTAGAGA |
| | | ATTTTT |
| SEQ ID NO: 66 | Panteoa agglomerans | TTTTTTAACCCTGTCGATTGACGC |
| | | CTTTTT |
| SEQ ID NO: 67 | Panteoa stewartii | TTTTTTAACCTCATCAATTGACGC |
| | | CTTTTT |
| SEQ ID NO: 68 | Pseudomonas aeruginosa | TTTTTGCAGTAAGTTAATACCTTG |
| | | TCTTTT |
| SEQ ID NO: 69 | Pseudomonas cannabina | TTTTTTTACGTATCTGTTTTGACT |
| | | CTTTTT |
| SEQ ID NO: 70 | Pseudomonas spp. 1 | TTTTTTGTTACCRACAGAATAAGC |
| | | ATTTTT |
| SEQ ID NO: 71 | Pseudomonas spp. 2 | TTTTTTAAGCACTTTAAGTTGGGA |
| | | TTTTTT |
| SEQ ID NO: 72 | Pseudomonas spp. 3 | TTTATTTTAAGCACTTTAAGTTGG |
| | | GATTTTATTT |
| SEQ ID NO: 73 | Salmonella bongori | TTTTTTTAATAACCTTGTTGATTG |
| | | TTTTTT |
| SEQ ID NO: 74 | Salmonella | TTTTTTTGTTGTGGTTAATAACCG |
| | enterica/Enterobacter 1 | ATTTTT |
| SEQ ID NO: 75 | Salmonella | TTTTTTTAACCGCAGCAATTGACT |
| | enterica/Enterobacter 2 | CTTTTT |
| SEQ ID NO: 76 | Salmonella | TTTTTTCTGTTAATAACCGCAGCT |
| | enterica/Enterobacter 3 | TTTTTT |
| SEQ ID NO: 77 | Serratia spp. | TTTATTCTGTGAACTTAATACGTT |
| | | CATTTTTATT |
| SEQ ID NO: 78 | Staphylococcus aureus 1 | TTTATTTTCATATGTGTAAGTAAC |
| | | TGTTTTATTT |
| SEQ ID NO: 79 | Staphylococcus aureus 2 | TTTTTTCATATGTGTAAGTAACTG |
| | | TTTTTT |
| SEQ ID NO: 80 | Streptomyces spp. | TTTTATTTTAAGAAGCGAGAGTGA |
| | | CTTTTATTTT |
| SEQ ID NO: 81 | stx1 gene | TTTTTTCTTTCCAGGTACAACAGC |
| | | TTTTTT |
| SEQ ID NO: 82 | stx2 gene | TTTTTTGCACTGTCTGAAACTGCC |
| | | TTTTTT |
| SEQ ID NO: 83 | Vibrio spp. | TTTTTTGAAGGTGGTTAAGCTAAT |
| | | TTTTTT |
| SEQ ID NO: 84 | Xanthamonas spp. | TTTTTTGTTAATACCCGATTGTTC |
| | | TTTTTT |
| SEQ ID NO: 85 | Yersinia pestis | TTTTTTTGAGTTTAATACGCTCAA |
| | | CTTTTT |

TABLE 8
Calibration and Negative Controls

| SEQ ID NO: | Imager | TTTTCTATGTATCGATGTTGAGAAAT |
|---|---|---|
| 129 | Calibration | TTTTTT |
| | (High) | |
| SEQ ID NO: | Imager | TTTTCTAGATACTTGTGTAAGTGAAT |
| 130 | Calibration | TTTTTT |
| | (Low) | |
| SEQ ID NO: | Imager | TTTTCTAAGTCATGTTGTTGAAGAAT |
| 131 | Calibration | TTTTTT |
| | (Medium) | |
| SEQ ID NO: | Negative | TTTTTTCTACTACCTATGCTGATTCA |
| 132 | control | CTCTTTTT |

TABLE 9
Oligonucleotide probe sequence for the ITS2 Locus

| | | |
|---|---|---|
| SEQ ID NO: 86 | Total Yeast and | TTTTTTTTGAATCATCGARTCTTTGAACG |
| | Mold (High | CATTTTTTT |
| | sensitivity) | |
| SEQ ID NO: 87 | Total Yeast and | TTTTTTTTGAATCATCGARTCTCCTTTTTT |
| | Mold (Low | T |
| | sensitivity) | |
| SEQ ID NO: 88 | Total Yeast and | TTTTTTTTGAATCATCGARTCTTTGAACG |
| | Mold (Medium | TTTTTTT |
| | sensitivity) | |
| SEQ ID NO: 89 | Alternaria spp. | TTTTTTCAAAGGTCTAGCATCCATTAAGT |
| | | TTTTT |
| SEQ ID NO: 90 | Aspergillus flavus 1 | TTTTTTCGCAAATCAATCTTTTTCCAGTCT |
| | | TTTT |
| SEQ ID NO: 91 | Aspergillus flavus 2 | TTTTTTTCTTGCCGAACGCAAATCAATCT |
| | | TTTTTTTTTTT |
| SEQ ID NO: 92 | Aspergillus | TTTCTTTTCGACACCCAACTTTATTTCCTT |
| | fumigatus 1 | ATTT |
| SEQ ID NO: 93 | Aspergillus | TTTTTTTGCCAGCCGACACCCATTCTTTT |
| | fumigatus 2 | T |
| SEQ ID NO: 94 | Aspergillus | TTTTTTGGCGTCTCCAACCTTACCCTTTT |
| | nidulans | T |
| SEQ ID NO: 95 | Aspergillus niger 1 | TTTTTTCGACGTTTTCCAACCATTTCTTTT |
| SEQ ID NO: 96 | Aspergillus niger 2 | TTTTTTTTCGACGTTTTCCAACCATTTCTT |
| | | TTTT |
| SEQ ID NO: 97 | Aspergillus niger 3 | TTTTTTTCGCCGACGTTTTCCAATTTTTTT |
| SEQ ID NO: 98 | Aspergillus terreus | TTTTTCGACGCATTTATTTGCAACCCTTT |
| | | T |
| SEQ ID NO: 99 | Blumeria | TTTATTTGCCAAAAMTCCTTAATTGCTCT |
| | | TTTTT |
| SEQ ID NO: 100 | Botrytis spp | TTTTTTTCATCTCTCGTTACAGGTTCTCG |
| | | GTTCTTTTTTT |
| SEQ ID NO: 101 | Candida albicans | TTTTTTTTTGAAAGACGGTAGTGGTAAGT |
| | | TTTTT |
| SEQ ID NO: 102 | Candida spp. | TTTTTTTGTTTGGTGTTGAGCRATACGTA |
| | Group 1 | TTTTT |
| SEQ ID NO: 103 | Candida spp. | TTTTACTGTTTGGTAATGAGTGATACTCT |
| | Group 2 | CATTTT |
| SEQ ID NO: 104 | Chaetomium spp. | TTTCTTTTGGTTCCGGCCGTTAAACCATT |
| | | TTTTT |
| SEQ ID NO: 105 | Cladosporium spp | TTTTTTTTGTGGAAACTATTCGCTAAAGT |
| | | TTTTT |
| SEQ ID NO: 106 | Erysiphe spp. | TTTCTTTTTACGATTCTCGCGACAGAGTT |
| | | TTTTT |
| SEQ ID NO: 107 | Fusarium | TTTTTTTCTCGTTACTGGTAATCGTCGTT |
| | oxysporum | TTTTT |
| SEQ ID NO: 108 | Fusarium spp | TTTTTTTTAACACCTCGCRACTGGAGATT |
| | | TTTTT |
| SEQ ID NO: 109 | Golovinomyces | TTTTTTCCGCTTGCCAATCAATCCATCTC |
| | | TTTTT |
| SEQ ID NO: 110 | Histoplasma | TTTATTTTTGTCGAGTTCCGGTGCCCTTT |
| | capsulatum | TATTT |
| SEQ ID NO: 111 | Isaria spp. | TTTATTTTTCCGCGGCGACCTCTGCTCTT |
| | | TATTT |
| SEQ ID NO: 112 | Monocillium spp. | TTTCTTTTGAGCGACGACGGGCCCAATT |
| | | TTCTTT |
| SEQ ID NO: 113 | Mucor spp. | TTTTCTCCAWTGAGYACGCCTGTTTCTTT |
| | | T |
| SEQ ID NO: 114 | Myrothecium spp. | TTTATTTTCGGTGGCCATGCCGTTAAATT |
| | | TTATT |
| SEQ ID NO: 115 | Oidiodendron spp. | TTTTTTTGCGTAGTACATCTCTCGCTCAT |
| | | TTTTT |
| SEQ ID NO: 116 | Penicillium | TTTTTTACACCATCAATCTTAACCAGGCC |
| | oxalicum | TTTTT |
| SEQ ID NO: 117 | Penicillium paxilli | TTTTTTCCCCTCAATCTTTAACCAGGCCT |
| | | TTTTT |
| SEQ ID NO: 118 | Penicillium spp | TTTTTTCAACCCAAATTTTTATCCAGGCC |
| | | TTTTT |
| SEQ ID NO: 119 | Phoma/Epicoccum | TTTTTTTGCAGTACATCTCGCGCTTTGAT |
| | spp. | TTTTT |
| SEQ ID NO: 120 | Podosphaera spp | TTTTTTGACCTGCCAAAACCCACATACCA |
| | | TTTTT |
| SEQ ID NO: 121 | Podosphaera spp. | TTTTTTTTAGTCAYGTATCTCGCGACAGT |
| | | TTTTT |
| SEQ ID NO: 122 | Pythium | TTTTATTTAAAGGAGACAACACCAATTTT |
| | oligandrum | TATTT |
| SEQ ID NO: 123 | Rhodoturula spp | TTTTTTCTCGTTCGTAATGCATTAGCACT |
| | | TTTTT |
| SEQ ID NO: 124 | Stachybotrys spp | TTTCTTCTGCATCGGAGCTCAGCGCGTT |
| | | TTATTT |
| SEQ ID NO: 125 | Trichoderma spp | TTTTTCCTCCTGCGCAGTAGTTTGCACAT |
| | | CTTTT |
| SEQ ID NO: 136 | Total Yeast and | TTTTTTTTGCATCATAGAAACTTTGTAC |
| | Mold Quantitative | GCATTT TTTT |
| | Control (internal | |
| | reference | |
| | standard) | |
| SEQ ID NO: 137 | Golovinomyces | TTTATTTAATCAATCCATCATCTCAAGT |
| | spp. | CTTTTT |
| SEQ ID NO: 138 | Mucor spp. | TTTTTTCTCCAWTGAGYACGCCTGTTTC |
| | | AGTAT CTTTTTT |
| SEQ ID NO: 139 | Aspergillus terreus | TTTTTTACGCATTTATTTGCAACTTGCCT |
| | | TTTTT |
| SEQ ID NO: 140 | Podosphaera spp. | TTTTTCGTCCCCTAAACATAGTGGCTTT |
| | | TT |

Table 11 displays representative oligonucleotide sequences which are used as microarray probes in an embodiment for DNA microarray-based analysis of bacterial pathogens (stx1, stx2, invA, tuf) and for DNA analysis of the presence host Cannabis at the ITS1 locus (Cannabis spp.). It should be noted that this same approach, with modifications to the ITS1 sequence, could be used to analyze the presence of other plant hosts in such extracts.

TABLE 10
Oligonucleotide probe sequence for
the Cannabis ITS1 Locus

| | | |
|---|---|---|
| SEQ ID | Cannabis ITS1 | TTTTTTAATCTGCGCCAAGGAACAATA |
| NO: 126 | DNA Control 1 | TTTTTTT |
| SEQ ID | Cannabis ITS1 | TTTTTGCAATCTGCGCCAAGGAACAAT |
| NO: 127 | DNA Control 2 | ATTTTTT |
| SEQ ID | Cannabis ITS1 | TTTATTTCTTGCGCCAAGGAACAATAT |
| NO: 128 | DNA Control 3 | TTTATTT |

TABLE 11
Representative Microarray Probe Design
for the Present Invention:
Bacterial Toxins, ITS1 (Cannabis)

| SEQ ID NO: 81 | stx1 gene | TTTTTTCTTTCCAGGTACAACAG |
|---|---|---|
| | | CTTTTTT |
| SEQ ID NO: 82 | stx2 gene | TTTTTTGCACTGTCTGAAACTGC |
| | | CTTTTTT |
| SEQ ID NO: 59 | etuf gene | TTTTTTCCATCAAAGTTGGTGAA |
| | | GAATCTTTTTT |
| SEQ ID NO: 61 | invA gene | TTTTTTTATTGATGCCGATTTGA |
| | | AGGCCTTTTTT |
| SEQ ID NO: | Cannabis ITS1 | TTTTTTAATCTGCGCCAAGGAAC |
| 126 | DNA Control 1 | AATATTTTTTT |

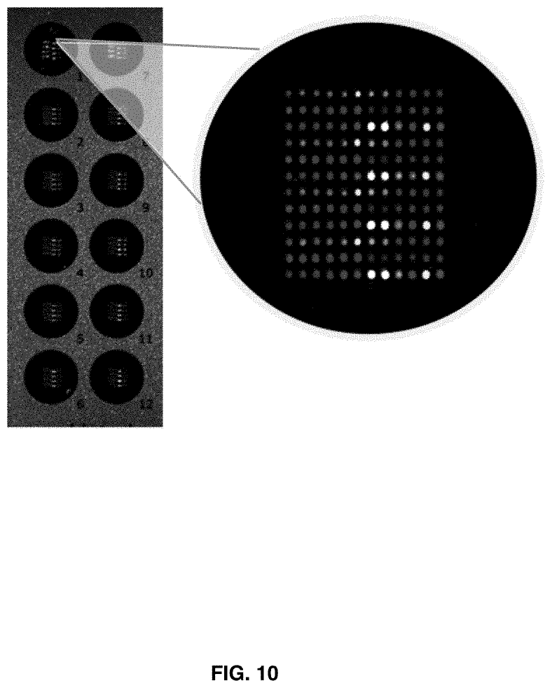

FIG. 9 shows a flow diagram to describe how an embodiment is used to analysis the bacterial pathogen or yeast and mold complement of a Cannabis or related plant sample. Pathogen samples can be harvested from Cannabis plant material by tape pulling of surface bound pathogen or by simple washing of the leaves or buds or stems, followed by a single multiplex “Loci Enhancement” Multiplex PCR reaction, which is then followed by a single multiplex “Labelling PCR”. A different pair of two step PCR reactions is used to analyze bacteria, than the pair of two step PCR reactions used to analyze fungi, yeast & mold. In all cases, the DNA of the target bacteria or fungi, yeast & mold are PCR amplified without extraction or characterization of the DNA prior to two step PCR. Subsequent to the Loci Enhancement and Labelling PCR steps, the resulting PCR product is simply diluted into binding buffer and then applied to the microarray test. The subsequent microarray steps required for analysis (hybridization and washing) are performed at lab ambient temperature. FIG. 10 provide images of a representative implementation of microarrays used in an embodiment. In this implementation, all nucleic acid probes required for bacterial analysis, along with Cannabis DNA controls (Tables 7 and 10) are fabricated into a single 144 element (12×12) microarray, along with additional bacterial and Cannabis probes such as those in Table 10. In this implementation, all nucleic acid probes required for fungi, yeast & mold analysis along with Cannabis DNA controls were fabricated into a single 144 element (12×12) microarray, along with additional fungal probes shown in Table 9. The arrays are manufactured on PTFE coated glass slides as two columns of 6 identical microarrays. Each of the 12 identical microarrays is capable of performing, depending on the nucleic acid probes employed, a complete microarray-based analysis bacterial analysis or a complete microarray-based analysis of fungi, yeast & mold. Nucleic acid probes were linked to the glass support via microfluidic printing, either piezoelectric or contact based or an equivalent. The individual microarrays are fluidically isolated from the other 11 in this case, by the hydrophobic PTFE coating, but other methods of physical isolation can be employed.

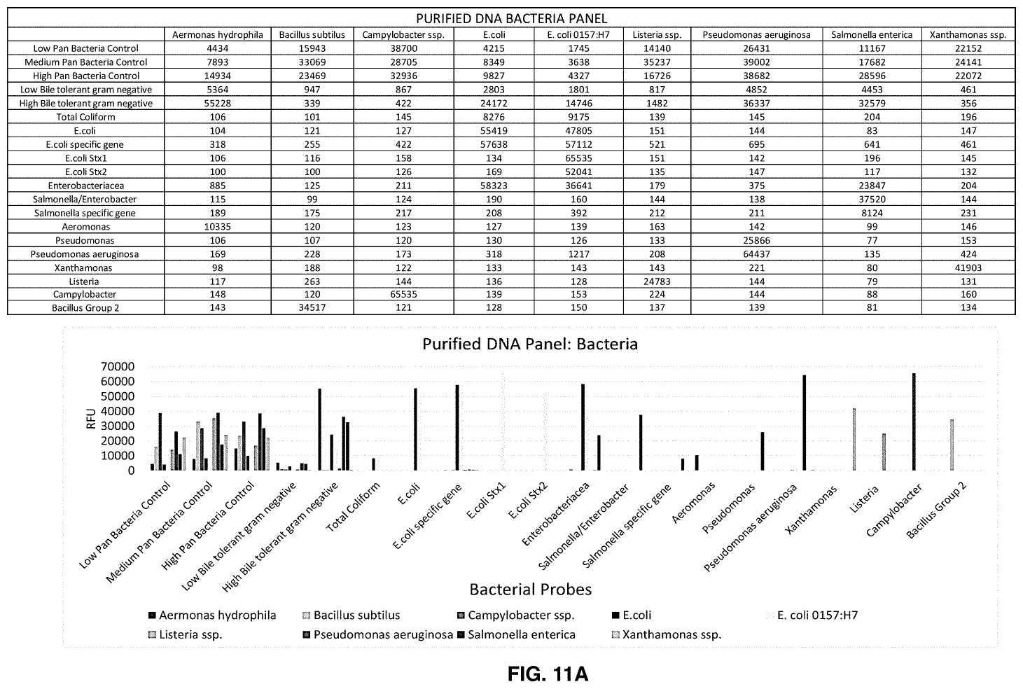

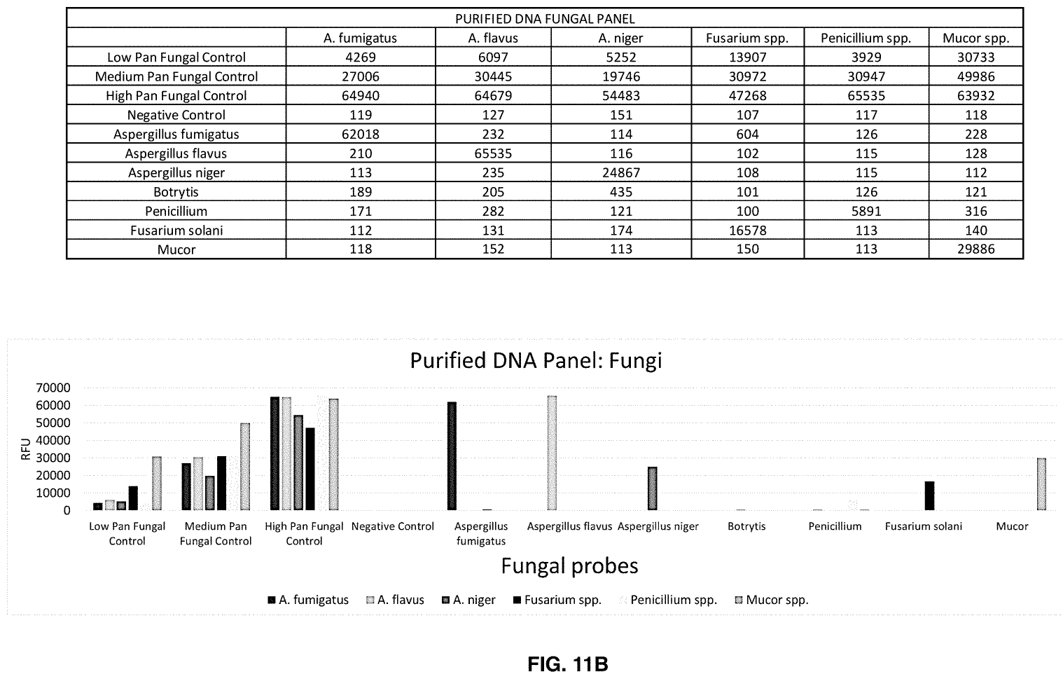

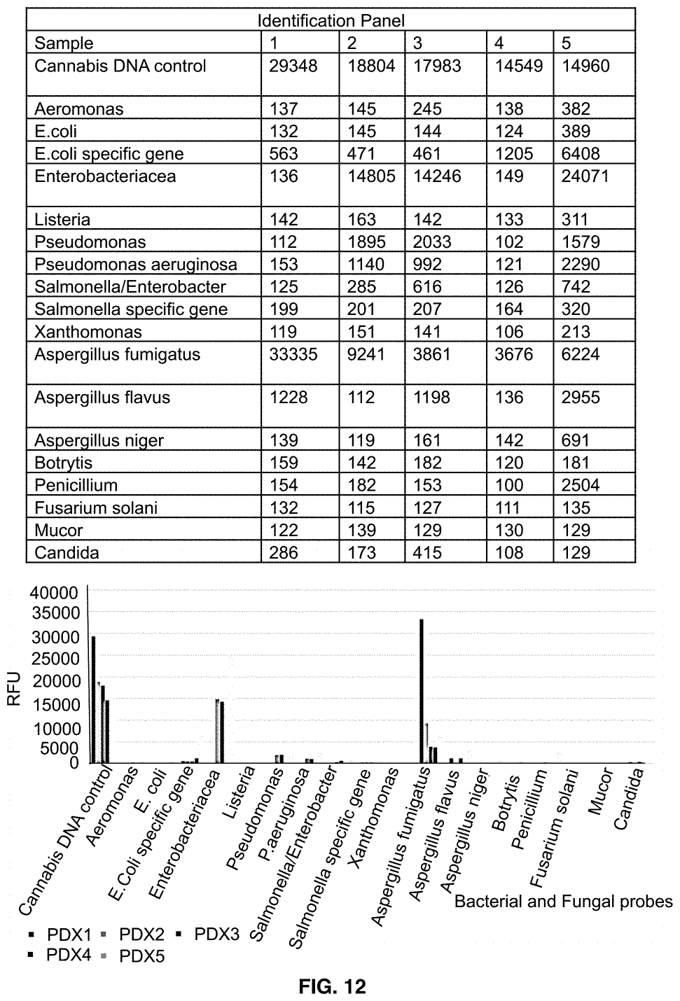

FIGS. 11A-11B display representative DNA microarray analysis of an embodiment. In this case, purified bacterial DNA or purified fungal DNA has been used, to test for affinity and specificity subsequent to the two-step PCR reaction and microarray-based hybridization analysis. As can be seen, the nucleic acid probes designed to detect each of the bacterial DNA (top) or fungal DNA (bottom) have bound to the target DNA correctly via hybridization and thus have correctly detected the bacterium or yeast. FIG. 12 displays representative DNA microarray analysis of an embodiment. In this case, 5 different unpurified raw Cannabis leaf wash samples were used to test for affinity and specificity subsequent to the two-step PCR reaction and microarray-based hybridization analysis. Both bacterial and fungal analysis has been performed on all 5 leaf wash samples, by dividing each sample into halves and subsequently processing them each for analysis of bacteria or for analysis of fungi, yeast & mold. The data of FIG. 12 were obtained by combining the outcome of both assays. FIG. 12 shows that the combination of two step PCR and microarray hybridization analysis, as described in FIG. 9, can be used to analyze the pathogen complement of a routine Cannabis leaf wash. It is expected, but not shown that such washing of any plant material could be performed similarly.

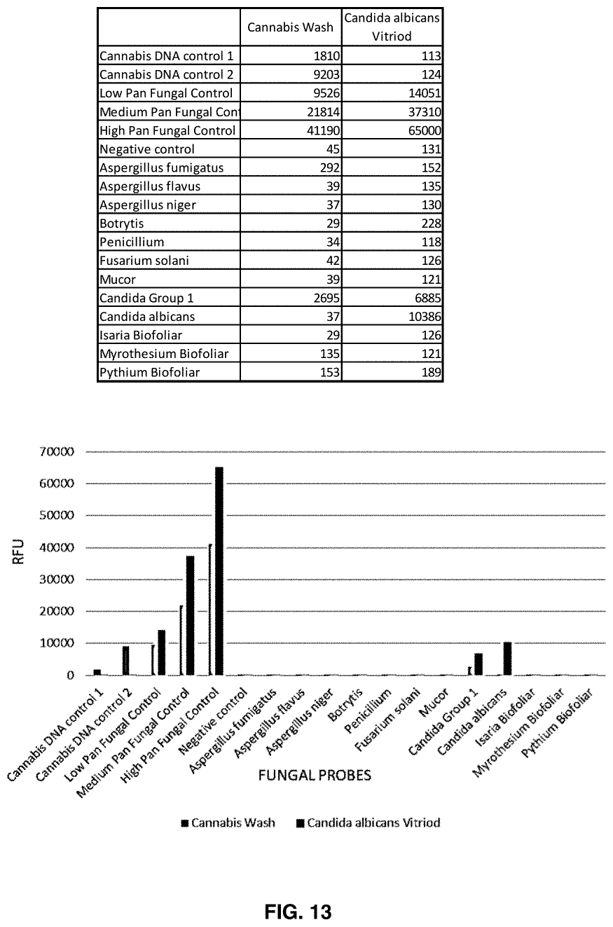

FIG. 13 displays representative DNA microarray analysis of an embodiment. In this case, one unpurified (raw) Cannabis leaf wash sample was used and was compared to data obtained from a commercially-obtained homogenous yeast vitroid culture of live Candida to test for affinity and specificity subsequent to the two-step PCR reaction and microarray-based hybridization analysis. Both Cannabis leaf wash and cultured fungal analysis have been performed by processing them each for analysis via probes specific for fungi (see Tables 9 and 11).

The data of FIG. 13 were obtained by combining the outcome of analysis of both the leaf wash and yeast vitroid culture samples. The data of FIG. 13 show that the combination of two step PCR and microarray hybridization analysis, as described in FIG. 9, can be used to interrogate the fungal complement of a routine Cannabis leaf wash as adequately as can be done with a pure (live) fungal sample. It is expected that fungal analysis via such washing of any plant material could be performed similarly.

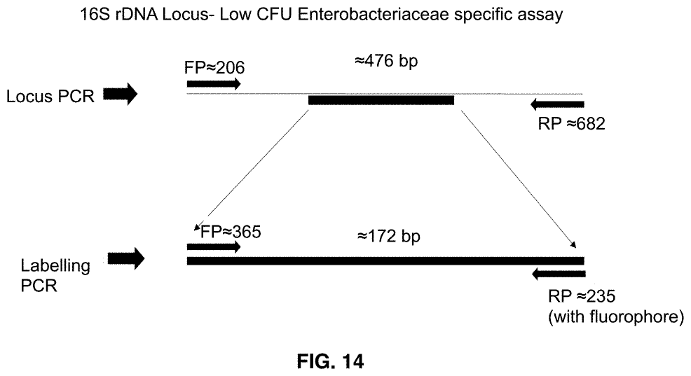

FIG. 14 shows a graphical representation of the position of PCR primers employed in a variation of an embodiment for low level detection of Bacteria in the Family Enterobacteriaceae including E. coll. These PCR primers are used to selectively amplify and dye label DNA from targeted organisms for analysis via microarray hybridization.

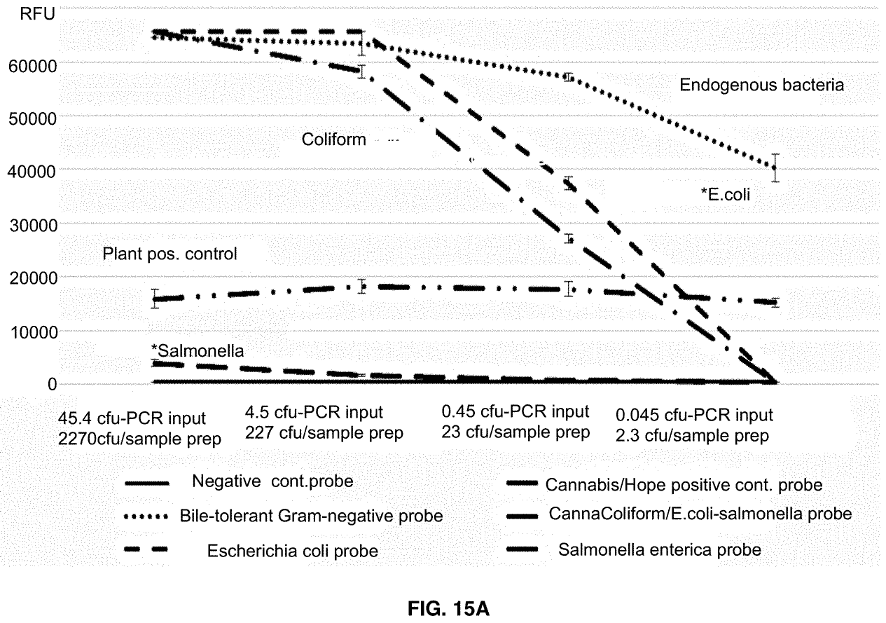

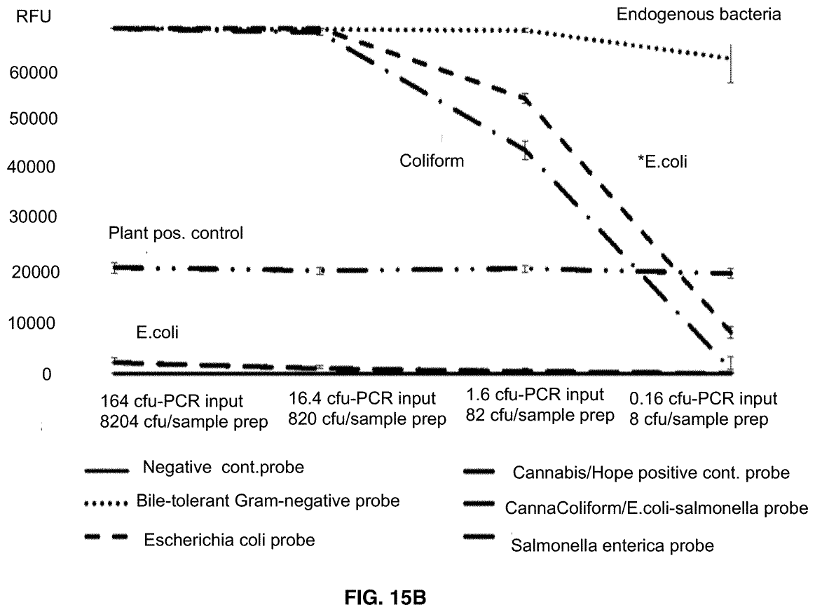

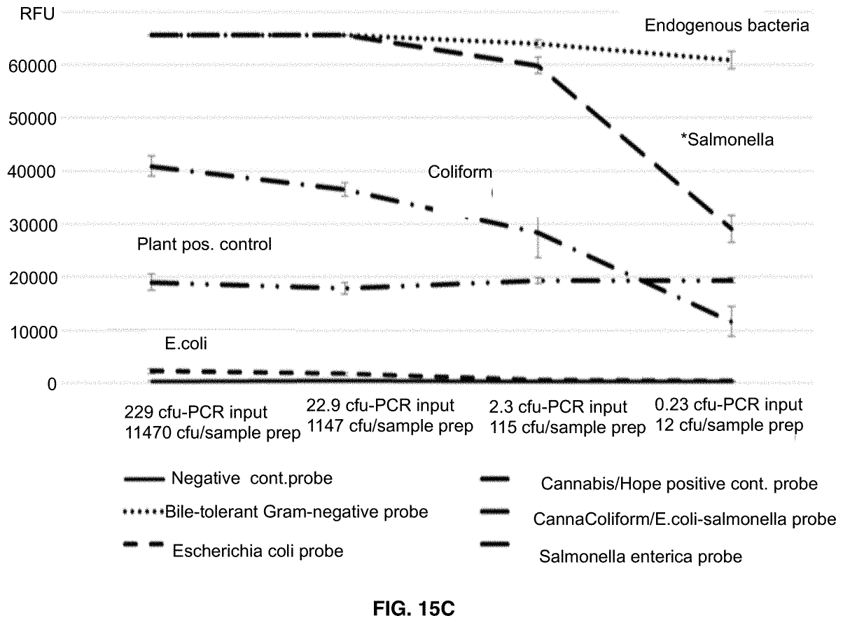

FIGS. 15A-15C illustrate representative DNA microarray analysis demonstrating assay sensitivity over a range of microbial inputs. In this case, certified reference material consisting of enumerated bacterial colonies of E. coli O157:H7, E. coli O111 (FIGS. 15A, 15B) and Salmonella enterica (FIG. 15C) were spiked as a dilution series onto a hops plant surrogate matrix then processed using the assay version described for FIG. 14. Hybridization results from relevant probes from FIG. 7 are shown. The larger numbers on the x-axis represents the total number of bacterial colony forming units (CFU) that were spiked onto each hops plant sample, whereas the smaller numbers on the x-axis represent the number of CFU's of the spiked material that were actually inputted into the assay. Only about 1/50 of the original spiked hops sample volume was actually analyzed. The smaller numbers upon the x-axis of FIGS. 15A-15C are exactly 1/50^{th} that of the total (lower) values. As is seen, FIGS. 15A-15C show that the microarray test of an embodiment can detect less than 1 CFU per microarray assay. The nucleic acid targets within the bacterial genomes displayed in FIGS. 15A-15C comprise 16S rDNA. There are multiple copies of the 16S rDNA gene in each of these bacterial organisms, which enables detection at <1 CFU levels. Since a colony forming unit approximates a single bacterium in many cases, the data of FIGS. 15A-15C demonstrate that the present microarray assay has sensitivity which approaches the ability to detect a single (or a very small number) of bacteria per assay. Similar sensitivity is expected for all bacteria and eukaryotic microbes in that it is known that they all present multiple copies of the ribosomal rDNA genes per cell.

Tables 12A and 12B show a collection of representative microarray hybridization data obtained from powdered dry food samples with no enrichment and 18-hour enrichment for comparison. The data shows that bacterial microbes were successfully detected on the microarrays of the present invention without the need for enrichment.

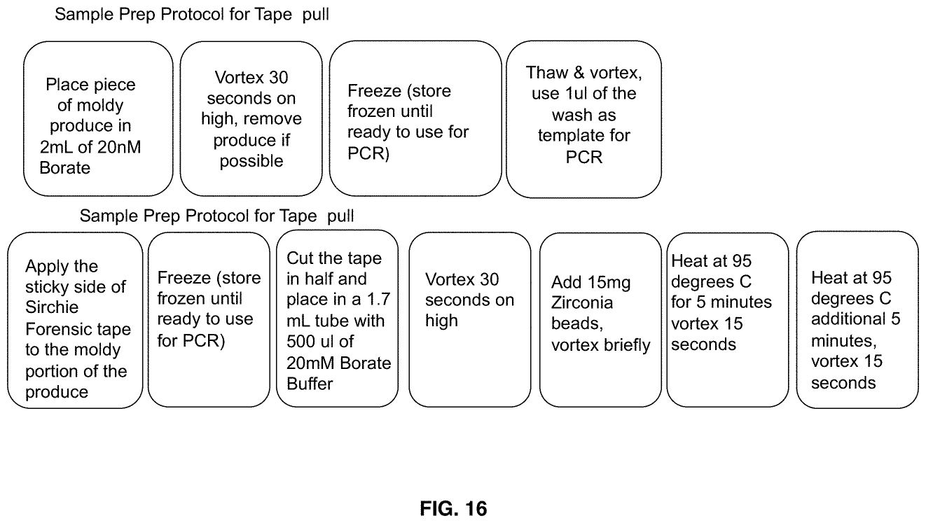

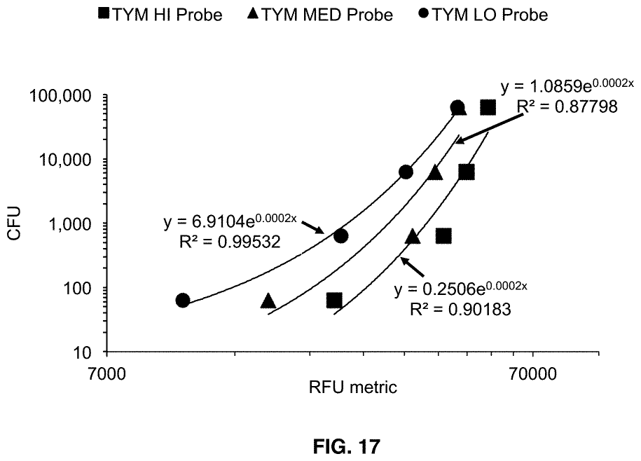

FIG. 16 and Tables 13-15 describes embodiments for the analysis of fruit, embodiments for the analysis of vegetables and embodiments for the analysis of other plant matter. The above teaching shows, by example, that unprocessed leaf and bud samples in Cannabis and hops may be washed in an aqueous water solution, to yield a water-wash containing microbial pathogens which can then be analyzed via the present combination of RSG and microarrays.

If fresh leaf, flower, stem or root materials from fruit and vegetables are also washed in a water solution in that same way (when fresh, or after drying and grinding or other types or processing, then the present combination of RSG and microarray analysis would be capable of recovering and analyzing the DNA complement of those microbes in those other plant materials.

At least two methods of sample collection are possible for fruit and vegetables. One method is the simple rinsing of the fruit, exactly as described for Cannabis, above. Another method of sample collection from fruits and vegetables is a “tape pull”, wherein a piece of standard forensic tape is applied to the surface of the fruit, then pulled off. Upon pulling, the tape is then soaked in the standard wash buffer described above, to suspend the microbes attached to the tape. Subsequent to the tape-wash step, all other aspects of the processing and analysis (i.e., raw sample genotyping, PCR, then microarray analysis) are exactly as described above.

TABLE 12A
Representative microarray data obtained from powdered dry food samples.

| | Sample Type | | | | | | | | | |
|---|---|---|---|---|---|---|---|---|---|---|
| | Whey Protein | | Whey Protein | | Chewable | | | | | |
| | Shake | | Shake | | Berry | | Vanilla | | | |
| | Vanilla | | Chocolate | | Tablet | | Shake | | Pea Protein | |
| | Enrichment time (hours) | | | | | | | | | |
| | 0 | 18 | 0 | 18 | 0 | 18 | 0 | 18 | 0 | 18 |
| Negative Control | 289 | 318 | 349 | 235 | 327 | 302 | 358 | 325 | 321 | 299 |
| Probe | | | | | | | | | | |
| Total Aerobic | | | | | | | | | | |
| Bacteria Probes | | | | | | | | | | |
| High sensitivity | 26129 | 38896 | 16629 | 11901 | 3686 | 230 | 32747 | 12147 | 41424 | 40380 |
| Medium sensitivity | 5428 | 6364 | 3308 | 2794 | 876 | 215 | 7310 | 2849 | 15499 | 8958 |
| Low sensitivity | 2044 | 3419 | 1471 | 990 | 446 | 181 | 2704 | 1062 | 4789 | 3887 |
| Bile-tolerant | | | | | | | | | | |
| Gram-negative | | | | | | | | | | |
| Probes | | | | | | | | | | |
| High sensitivity | 2639 | 350 | 1488 | 584 | 307 | 305 | 1041 | 472 | 15451 | 8653 |
| Medium sensitivity | 1713 | 328 | 892 | 493 | 322 | 362 | 615 | 380 | 6867 | 4997 |
| Low sensitivity | 974 | 600 | 749 | 621 | 595 | 688 | 821 | 929 | 2459 | 1662 |
| Total | | | | | | | | | | |
| Enterobacteriaceae | | | | | | | | | | |
| Probes | | | | | | | | | | |
| High sensitivity | 1131 | 306 | 363 | 310 | 346 | 318 | 273 | 331 | 4260 | 3149 |
| Medium sensitivity | 479 | 296 | 320 | 297 | 329 | 339 | 314 | 342 | 1489 | 990 |
| Low sensitivity | 186 | 225 | 203 | 165 | 205 | 181 | 207 | 200 | 216 | 259 |
| 16S rDNA | | | | | | | | | | |
| Species Probes | | | | | | | | | | |
| Escherichia | 233 | 205 | 255 | 219 | 207 | 255 | 215 | 214 | 242 | 198 |
| coli/Shigella spp. | | | | | | | | | | |
| S. enterica/ | 203 | 183 | 186 | 281 | 212 | 299 | 197 | 257 | 308 | 303 |
| enterobacter spp. | | | | | | | | | | |
| Bacillus spp. | 154 | 172 | 189 | 114 | 307 | 156 | 169 | 153 | 233 | 259 |
| Pseudomonas | 549 | 201 | 202 | 251 | 148 | 216 | 303 | 276 | 2066 | 983 |
| spp. | | | | | | | | | | |
| Organism Specific | | | | | | | | | | |
| Gene Probes | | | | | | | | | | |
| tuf gene(E. coli) | 204 | 129 | 180 | 272 | 158 | 190 | 191 | 183 | 186 | 192 |
| stx1 gene(E. coli) | 241 | 178 | 171 | 240 | 289 | 304 | 195 | 245 | 149 | 191 |
| stx2 gene(E. coli) | 145 | 96 | 136 | 125 | 182 | 224 | 130 | 142 | 85 | 127 |
| invA (Salmonella | 215 | 265 | 210 | 284 | 204 | 256 | 239 | 285 | 237 | 229 |
| spp.) | | | | | | | | | | |

TABLE 12B
Representative microarray data obtained from powdered dry food samples.

| | Sample Type | | | | | | | |
|---|---|---|---|---|---|---|---|---|
| | | | Work-out | | Work-out | | | |
| | Rice Protein | | Shake FP | | Shake BR | | Vanilla Shake | |
| | Enrichment time (hours) | | | | | | | |
| | 0 | 18 | 0 | 18 | 0 | 18 | 0 | 18 |
| Negative Control | 351 | 351 | 271 | 309 | 299 | 332 | 246 | 362 |
| Probe | | | | | | | | |
| Total Aerobic | | | | | | | | |
| Bacteria Probes | | | | | | | | |
| High sensitivity | 471 | 288 | 17146 | 266 | 19207 | 261 | 41160 | 47198 |
| Medium sensitivity | 161 | 187 | 3120 | 229 | 3309 | 311 | 10060 | 22103 |
| Low sensitivity | 186 | 239 | 1211 | 261 | 1223 | 264 | 3673 | 6750 |
| Bile-tolerant | | | | | | | | |
| Gram-negative | | | | | | | | |
| Probes | | | | | | | | |
| High sensitivity | 326 | 372 | 375 | 380 | 412 | 363 | 1418 | 358 |
| Medium | 304 | 362 | 341 | 391 | 308 | 356 | 699 | 394 |
| sensitivity | | | | | | | | |
| Low sensitivity | 683 | 942 | 856 | 689 | 698 | 864 | 848 | 665 |
| Total | | | | | | | | |
| Enterobacteriaceae | | | | | | | | |
| Probes | | | | | | | | |
| High sensitivity | 277 | 329 | 317 | 327 | 298 | 326 | 290 | 349 |
| Medium sensitivity | 326 | 272 | 296 | 291 | 297 | 263 | 262 | 307 |
| Low sensitivity | 215 | 207 | 204 | 288 | 213 | 269 | 195 | 247 |
| 16S rDNA | | | | | | | | |
| Species Probes | | | | | | | | |
| Escherichia | 228 | 229 | 216 | 267 | 221 | 253 | 220 | 207 |
| coli/Shigella spp. | | | | | | | | |
| S. enterica/ | 226 | 281 | 238 | 268 | 197 | 254 | 255 | 216 |
| enterobacter spp. | | | | | | | | |
| Bacillus spp. | 157 | 166 | 812 | 208 | 915 | 216 | 415 | 168 |
| Pseudomonas | 199 | 225 | 247 | 251 | 211 | 259 | 277 | 225 |
| spp. | | | | | | | | |
| Organism Specific | | | | | | | | |
| Gene Probes | | | | | | | | |
| tuf gene(E. coli) | 150 | 149 | 126 | 206 | 163 | 212 | 215 | 166 |
| stx1 gene(E. coli) | 270 | 247 | 211 | 299 | 239 | 307 | 175 | 185 |
| stx2 gene(E. coli) | 158 | 178 | 127 | 205 | 138 | 175 | 128 | 100 |
| invA (Salmonella | 257 | 241 | 249 | 264 | 220 | 258 | 239 | 245 |
| spp.) | | | | | | | | |

The data of Tables 13-15 demonstrates that simple washing of the fruit and tape pull sampling of the fruit generate similar microbial data. The blueberry sample is shown to be positive for Botrytis, as expected, since Botrytis is a well-known fungal contaminant on blueberries. The lemon sample is shown to be positive for Penicillium, as expected, since Penicillium is a well-known fungal contaminant for lemons.

TABLE 13
Representative microarray hybridization data
obtained from blueberry and lemon washes.

| | Sample | | | |
|---|---|---|---|---|
| | Blueberry | | Lemon | |
| | Collection Type | | | |
| | Produce Wash | | | |
| | Protocol | | | |
| | | | Wash 1 piece moldy | |
| | Wash 1 blueberry in 2 ml | | lemon in 2 ml 20 mM | |
| | 20 mM Borate, vortex 30 | | Borate, vortex 30 | |
| | seconds | | seconds | |
| Dilution Factor | NONE | 1:20 | NONE | 1:20 |
| A. fumigatus 1 | 65 | 61 | 62 | 57 |
| A. fumigatus 2 | 66 | 61 | 58 | 131 |
| A. fumigatus 3 | 69 | 78 | 55 | 127 |
| A. fumigatus 4 | 80 | 198 | 63 | 161 |
| A. fumigatus 5 | 98 | 68 | 59 | 70 |
| A. flavus 1 | 111 | 65 | 197 | 58 |
| A. flavus 2 | 64 | 66 | 71 | 49 |
| A. flavus 3 | 72 | 79 | 54 | 49 |
| A. flavus 4 | 95 | 71 | 66 | 125 |
| A. flavus 5 | 59 | 55 | 45 | 47 |
| A. niger 1 | 91 | 75 | 61 | 61 |
| A. niger 2 | 185 | 68 | 61 | 57 |
| A. niger 3 | 93 | 66 | 62 | 61 |
| A. niger 4 | 1134 | 74 | 75 | 64 |
| Botrytis spp. 1 | 26671 | 27605 | 60 | 55 |
| Botrytis spp. 2 | 26668 | 35611 | 59 | 57 |
| Penicillium spp. 1 | 63 | 69 | 2444 | 4236 |
| Penicillium spp. 2 | 71 | 69 | 4105 | 7426 |
| Fusarium spp. 1 | 175 | 69 | 59 | 78 |
| Fusarium spp. 2 | 71 | 73 | 84 | 62 |
| Mucor spp. 1 | 71 | 57 | 58 | 61 |
| Mucor spp. 2 | 61 | 290 | 66 | 61 |
| Total Y & M 1 | 20052 | 21412 | 8734 | 7335 |
| Total Y & M 2 | 17626 | 8454 | 5509 | 5030 |

The data embodied in FIG. 16 and Tables 13-15 demonstrate the use of an embodiment, for the recovery and analysis of yeast microbes on the surface of fruit (blueberries and lemons in this case), but an embodiment could be extended to other fruits and vegetables for the analysis of both bacterial and fungal contamination.

TABLE 14
Representative microarray hybridization data obtained from blueberry washes and tape pulls.

| Sample | Moldy Blueberry | | | | | | | | | | | |
|---|---|---|---|---|---|---|---|---|---|---|---|---|
| Collection Type | Tape Pull | | | | | | | | | | | |
| ID | 1A1 | 1A1 | 1A2 | 1A2 | 1A3 | 1A3 | 1B1 | 1B1 | 1B2 | 1B2 | 1B3 | 1B3 |
| Collection Point 1 | 500 ul 20 mM Borate Buffer, vortex 30 seconds | | | | | | 500 ul 20 mM Borate + Triton Buffer, vortex 30 seconds | | | | | |
| Collection Point 2 | | | Add 15 mg zirconia beads, vortex, | | | | | | Add 15 mg zirconia beads, vortex, | | | |
| | | | Heat 5 min 95° C., Vortex 15 seconds | | | | | | Heat 5 min 95° C., Vortex 15 seconds | | | |
| Collection Point 3 | | | | | Heat 5 min 95° C. | | | | | | Heat 5 min 95° C. | |
| | | | | | vortex 15 seconds | | | | | | vortex 15 seconds | |
| Dilution Factor | NONE | 1:20 | NONE | 1:20 | NONE | 1:20 | NONE | 1:20 | NONE | 1:20 | NONE | 1:20 |
| A. fumigatus 1 | 66 | 388 | 83 | 77 | 97 | 313 | 95 | 68 | 76 | 55 | 75 | 60 |
| A. fumigatus 2 | 97 | 100 | 82 | 118 | 69 | 56 | 87 | 67 | 185 | 76 | 58 | 52 |
| A. fumigatus 3 | 77 | 94 | 82 | 1083 | 87 | 61 | 93 | 84 | 75 | 378 | 73 | 64 |
| A. fumigatus 4 | 84 | 151 | 94 | 118 | 96 | 80 | 115 | 85 | 85 | 93 | 190 | 88 |
| A. fumigatus 5 | 63 | 75 | 96 | 71 | 78 | 61 | 98 | 74 | 68 | 98 | 70 | 533 |
| A. flavus 1 | 200 | 107 | 113 | 61 | 204 | 58 | 105 | 73 | 62 | 68 | 64 | 65 |
| A. flavus 2 | 70 | 104 | 64 | 57 | 133 | 281 | 111 | 78 | 377 | 314 | 57 | 50 |
| A. flavus 3 | 83 | 90 | 94 | 150 | 99 | 90 | 96 | 222 | 1162 | 86 | 80 | 73 |
| A. flavus 4 | 76 | 125 | 92 | 146 | 87 | 174 | 241 | 78 | 115 | 69 | 105 | 85 |
| A. flavus 5 | 80 | 153 | 77 | 72 | 78 | 439 | 71 | 86 | 280 | 58 | 62 | 57 |
| A. niger 1 | 409 | 178 | 122 | 72 | 80 | 70 | 76 | 71 | 152 | 117 | 65 | 53 |
| A. niger 2 | 78 | 292 | 79 | 65 | 715 | 666 | 74 | 70 | 68 | 731 | 70 | 54 |
| A. niger 3 | 86 | 76 | 87 | 558 | 78 | 60 | 70 | 81 | 96 | 63 | 478 | 58 |
| A. niger 4 | 164 | 70 | 92 | 108 | 197 | 69 | 130 | 75 | 76 | 148 | 73 | 65 |
| Botrytis spp. 1 | 41904 | 26549 | 28181 | 29354 | 25304 | 25685 | 57424 | 33783 | 57486 | 49803 | 33176 | 32153 |
| Botrytis spp. 2 | 36275 | 25518 | 29222 | 27076 | 26678 | 27675 | 49480 | 32899 | 52817 | 34322 | 29693 | 32026 |
| Penicillium spp. 1 | 80 | 81 | 83 | 64 | 96 | 60 | 79 | 80 | 176 | 60 | 385 | 53 |
| Penicillium spp. 2 | 90 | 93 | 81 | 80 | 114 | 59 | 98 | 69 | 470 | 65 | 478 | 56 |
| Fusarium spp. 1 | 77 | 71 | 69 | 62 | 112 | 55 | 61 | 274 | 617 | 81 | 59 | 757 |
| Fusarium spp. 2 | 91 | 82 | 107 | 74 | 101 | 65 | 91 | 66 | 123 | 63 | 71 | 583 |
| Mucor spp. 1 | 90 | 314 | 73 | 88 | 105 | 61 | 77 | 79 | 741 | 180 | 172 | 74 |
| Mucor spp. 2 | 83 | 69 | 73 | 69 | 91 | 67 | 111 | 102 | 455 | 88 | 70 | 133 |
| Total Y & M 1 | 23637 | 18532 | 15213 | 17668 | 18068 | 19762 | 18784 | 15550 | 20625 | 17525 | 25813 | 18269 |
| Total Y & M 2 | 12410 | 8249 | 9281 | 11526 | 8543 | 13007 | 14180 | 14394 | 9905 | 8972 | 15112 | 12678 |

TABLE 15
Representative microarray hybridization data obtained from lemon washes and tape pulls.

| Sample | Moldy Lemon | | | | |
|---|---|---|---|---|---|
| Collection Type | Tape Pull | | | | |
| ID | 1A1 Lemon | 1A2 Lemon | 1A3 Lemon | 1B1 Lemon | 1B2 Lemon |
| Collection Point 1 | 500 ul 20 mM Borate + Triton Buffer, vortex 30 seconds | | | | |
| Collection Point 2 | | Add 15 mg | | | Add 15 mg zirconia |
| | | zirconia beads, | | | beads, vortex, |
| | | vortex, Heat 5 | | | Heat 5 min 95° C., |
| | | min 95° C., Vortex | | | Vortex 15 seconds |
| | | 15 seconds | | | |
| Collection Point 3 | | | Heat 5 min | | |
| | | | 95° C. vortex | | |
| | | | 15 seconds | | |
| Dilution Factor | NONE | | | | |
| A. fumigatus 1 | 96 | 83 | 75 | 83 | 64 |
| A. fumigatus 2 | 221 | 73 | 71 | 66 | 101 |
| A. fumigatus 3 | 87 | 88 | 85 | 92 | 122 |
| A. fumigatus 4 | 83 | 85 | 91 | 72 | 97 |
| A. fumigatus 5 | 448 | 100 | 84 | 114 | 78 |
| A. flavus 1 | 85 | 79 | 70 | 66 | 63 |
| A. flavus 2 | 77 | 82 | 77 | 79 | 63 |
| A. flavus 3 | 133 | 66 | 86 | 60 | 67 |
| A. flavus 4 | 96 | 85 | 81 | 98 | 88 |
| A. flavus 5 | 68 | 62 | 65 | 106 | 59 |
| A. niger 1 | 73 | 88 | 77 | 73 | 73 |
| A. niger 2 | 74 | 84 | 81 | 71 | 103 |
| A. niger 3 | 90 | 86 | 87 | 74 | 78 |
| A. niger 4 | 82 | 93 | 104 | 86 | 161 |
| Botrytis spp. 1 | 82 | 75 | 75 | 77 | 68 |
| Botrytis spp. 2 | 91 | 74 | 83 | 67 | 62 |
| Penicillium spp. 1 | 3824 | 5461 | 5500 | 4582 | 5290 |
| Penicillium spp. 2 | 7586 | 8380 | 11177 | 6528 | 8167 |
| Fusarium spp. 1 | 101 | 62 | 61 | 70 | 279 |
| Fusarium spp. 2 | 77 | 122 | 78 | 68 | 233 |
| Mucor spp. 1 | 74 | 110 | 89 | 76 | 57 |
| Mucor spp. 2 | 132 | 1302 | 90 | 84 | 61 |
| Total Y & M 1 | 8448 | 12511 | 9249 | 12844 | 8593 |
| Total Y & M 2 | 9275 | 8716 | 11585 | 10758 | 4444 |

Table 16 shows embodiments for the analysis of environmental water samples/specimens. The above teaching shows by example that unprocessed leaf and bud samples in Cannabis and hops may be washed in an aqueous water solution, to yield a water-wash containing microbial pathogens which can then be analyzed via the present combination of Raw Sample Genotyping (RSG) and microarrays. If a water sample containing microbes were obtained from environmental sources (such as well water, or sea water, or soil runoff or the water from a community water supply) and then analyzed directly, or after ordinary water filtration to concentrate the microbial complement onto the surface of the filter, that the present combination of RSG and microarray analysis would be capable of recovering and analyzing the DNA complement of those microbes.

The data embodied in Table 16 were obtained from 5 well-water samples (named 2H, 9D, 21, 23, 25) along with 2 samples of milliQ laboratory water (obtained via reverse osmosis) referred to as “Negative Control”. All samples were subjected to filtration on a sterile 0.4 um filter. Subsequent to filtration, the filters, with any microbial contamination that they may have captured, were then washed with the standard wash solution, exactly as described above for the washing of Cannabis and fruit. Subsequent to that washing, the suspended microbes in wash solution were then subjected to exactly the same combination of centrifugation (to yield a microbial pellet) then lysis and PCR of the unprocessed pellet-lysate (exactly as described above for Cannabis), followed by PCR and microarray analysis, also as described for Cannabis.

TABLE 16
Representative microarray data from raw water filtrate.

| | | | | | | | | | | | Negative |
|---|---|---|---|---|---|---|---|---|---|---|---|
| Sample ID | 2H | 2H | 9D | 9D | 21 | 21 | 23 | 23 | 25 | 25 | Control |
| Imager Calibration High | 311 | 335 | 322 | 379 | 341 | 348 | 345 | 325 | 354 | 343 | 333 |
| Imager Calibration Med | 280 | 314 | 268 | 286 | 288 | 231 | 253 | 295 | 267 | 295 | 244 |
| Imager Calibration Low | 245 | 296 | 302 | 324 | 254 | 268 | 293 | 285 | 271 | 340 | 275 |
| Cannabis cont. | 310 | 330 | 313 | 255 | 323 | 368 | 313 | 322 | 274 | 332 | 322 |
| Cannabis cont. | 313 | 237 | 298 | 271 | 298 | 288 | 296 | 280 | 249 | 284 | 297 |
| Cannabis cont. | 208 | 265 | 276 | 250 | 267 | 327 | 255 | 258 | 253 | 282 | 370 |
| Total Yeast & Mold | 284 | 324 | 290 | 307 | 272 | 361 | 296 | 288 | 271 | 321 | 469 |
| Total Yeast & Mold | 251 | 259 | 294 | 290 | 309 | 308 | 285 | 281 | 275 | 299 | 293 |
| Total Yeast & Mold | 282 | 280 | 294 | 280 | 299 | 284 | 275 | 286 | 299 | 259 | 232 |
| Total Aerobic bacteria High | 40101 | 42007 | 47844 | 47680 | 45102 | 44041 | 43520 | 41901 | 46459 | 46783 | 135 |
| Total Aerobic bacteria Medium | 14487 | 12314 | 24189 | 26158 | 19712 | 16210 | 17943 | 15474 | 25524 | 18507 | 157 |
| Total Aerobic bacteria Low | 4885 | 5629 | 7625 | 6456 | 5807 | 4505 | 5316 | 6022 | 6264 | 6974 | 159 |
| Negative Control | 293 | 359 | 303 | 339 | 312 | 329 | 306 | 377 | 307 | 335 | 307 |
| Aspergillus fumigatus | 285 | 291 | 284 | 268 | 289 | 265 | 271 | 281 | 269 | 248 | 228 |
| Aspergillus flavus | 184 | 211 | 201 | 344 | 237 | 179 | 212 | 213 | 163 | 204 | 171 |
| Aspergillus niger | 226 | 213 | 228 | 273 | 190 | 195 | 245 | 206 | 222 | 209 | 172 |
| Botrytis spp. | 219 | 285 | 258 | 302 | 275 | 219 | 202 | 288 | 221 | 248 | 214 |
| Alternaria spp. | 81 | 97 | 76 | 89 | 58 | 76 | 75 | 175 | 117 | 174 | 167 |
| Penicillium paxilli | 135 | 162 | 215 | 142 | 127 | 161 | 103 | 115 | 238 | 190 | 200 |
| Penicillium oxalicum | 119 | 107 | 161 | 131 | 135 | 241 | 178 | 158 | 140 | 143 | 194 |
| Penicillium spp. | 50 | 123 | 179 | 177 | 128 | 138 | 146 | 163 | 148 | 115 | 184 |
| Can. alb/trop/dub | 261 | 236 | 235 | 230 | 250 | 213 | 276 | 244 | 245 | 237 | 194 |
| Can. glab/Sach & Kluv spp. | 146 | 165 | 196 | 128 | 160 | 215 | 185 | 217 | 215 | 177 | 225 |
| Podosphaera spp. | 111 | 119 | 100 | 122 | 192 | 105 | 95 | 43 | 169 | 27 | 143 |
| Bile-tolerant Gram-negative | 16026 | 9203 | 13309 | 8426 | 16287 | 14116 | 10557 | 17558 | 15343 | 14285 | 183 |
| High | | | | | | | | | | | |
| Bile-tolerant Gram-negative | 12302 | 11976 | 9259 | 10408 | 13055 | 10957 | 11242 | 8416 | 9322 | 11785 | 196 |
| Medium | | | | | | | | | | | |
| Bile-tolerant Gram-negative | 5210 | 7921 | 3818 | 3984 | 7224 | 6480 | 4817 | 6933 | 5021 | 5844 | 240 |
| Low | | | | | | | | | | | |
| Total Enterobacteriaceae High | 193 | 248 | 389 | 357 | 215 | 214 | 198 | 220 | 276 | 208 | 210 |
| Total Enterobacteriaceae Med | 246 | 214 | 297 | 246 | 244 | 224 | 219 | 245 | 252 | 229 | 207 |
| Total Enterobacteriaceae Low | 165 | 140 | 158 | 119 | 151 | 180 | 150 | 167 | 182 | 174 | 132 |
| Total Coliform | 121 | 148 | 158 | 117 | 129 | 117 | 155 | 157 | 125 | 178 | 152 |
| Escherichia coli specific gene | 31821 | 115 | 132 | 155 | 127 | 62 | 86 | 121 | 59 | 90 | 234 |
| stx1 gene | 67 | 0 | 2 | 0 | 0 | 23 | 21 | 28 | 0 | 0 | 116 |
| stx2 gene | 17 | 36 | 174 | 0 | 61 | 47 | 0 | 51 | 33 | 0 | 85 |
| Salmonella specific gene | 181 | 172 | 245 | 172 | 178 | 212 | 157 | 243 | 174 | 156 | 146 |
| Bacillus spp. | 137 | 135 | 174 | 112 | 164 | 143 | 163 | 182 | 168 | 152 | 149 |
| Pseudomonas spp. | 271 | 74 | 332 | 56 | 366 | 133 | 91 | 114 | 60 | 179 | 555 |
| Escherichia coli/Shigella spp. | 103 | 124 | 221 | 124 | 90 | 144 | 130 | 121 | 137 | 143 | 158 |
| Salmonella | 124 | 98 | 131 | 119 | 136 | 88 | 121 | 77 | 128 | 140 | 124 |
| enterica/enterobacter spp. | | | | | | | | | | | |
| Erysiphe Group 2 | 278 | 221 | 237 | 230 | 245 | 254 | 250 | 220 | 205 | 236 | 233 |
| Trichoderma spp. | 105 | 157 | 204 | 152 | 180 | 154 | 130 | 161 | 201 | 180 | 150 |
| Escherichia coli | 429 | 431 | 551 | 576 | 549 | 406 | 407 | 484 | 556 | 551 | 293 |
| Aspergillus niger | 218 | 212 | 216 | 297 | 255 | 312 | 221 | 202 | 238 | 231 | 209 |
| Escherichia coli/Shigella spp. | 163 | 193 | 220 | 202 | 308 | 280 | 121 | 271 | 341 | 317 | 124 |
| Aspergillus fumigatus | 713 | 865 | 862 | 830 | 784 | 657 | 827 | 803 | 746 | 812 | 793 |
| Aspergillus flavus | 155 | 261 | 198 | 156 | 239 | 171 | 250 | 218 | 210 | 258 | 219 |
| Salmonella enterica | 136 | 98 | 85 | 43 | 109 | 47 | 23 | 123 | 70 | 100 | 135 |
| Salmonella enterica | 68 | 53 | 52 | 41 | 60 | 92 | 26 | 28 | 55 | 81 | 116 |

The data seen in Table 16 demonstrate that microbes collected on filtrates of environmental water samples can be analyzed via the same combination of raw sample genotyping, then PCR and microarray analysis used for Cannabis and fruit washes. The italicized elements of Table 16 demonstrate that the 5 unprocessed well-water samples all contain aerobic bacteria and bile tolerant gram-negative bacteria. The presence of both classes of bacteria is expected for unprocessed (raw) well water. Thus, the data of Table 16 demonstrate that this embodiment of the present invention can be used for the analysis of environmentally derived water samples.

The above teaching shows that unprocessed leaf and bud samples in Cannabis and hops may be washed in an aqueous water solution to yield a water-wash containing microbial pathogens which can then be analyzed via the present combination of RSG and microarrays. The above data also show that environmentally-derived well water samples may be analyzed by an embodiment. Further, if a water sample containing microbes were obtained from industrial processing sources (such as the water effluent from the processing of fruit, vegetables, grain, meat) and then analyzed directly, or after ordinary water filtration to concentrate the microbial complement onto the surface of the filter, that the present combination of RSG and microarray analysis would be capable of recovering and analyzing the DNA complement of those microbes.

Further, if an air sample containing microbes as an aerosol or adsorbed to airborne dust were obtained by air filtration onto an ordinary air-filter (such as used in the filtration of air in an agricultural or food processing plant, or on factory floor, or in a public building or a private home) that such air-filters could then be washed with a water solution, as has been demonstrated for plant matter, to yield a microbe-containing filter eluate, such that the present combination of Raw Sample Genotyping (RSG) and microarray analysis would be capable of recovering and analyzing the DNA complement of those microbes.

While the foregoing written description of an embodiments enables one of ordinary skill to make and use what is considered presently to be the best mode thereof, those of ordinary skill will understand and appreciate the existence of variations, combinations, and equivalents of the specific embodiment, method, and examples herein. The present disclosure should therefore not be limited by the above described embodiments, methods, and examples, but by all embodiments and methods within the scope and spirit of the present disclosure.

### Example 4

PathogenDx QuantX assay for the detection of fungal contaminants in plants.

### Definitions

Probability of Detection (POD): The proportion of positive analytical outcomes for a qualitative method for a given matrix at a given analyte level or concentration. POD is concentration dependent. Several POD measures can be calculated; POD_{R} (reference method POD), POD_{C} (confirmed candidate method POD), POD_{CP} (candidate method presumptive result POD) and POD_{CC} (candidate method confirmation result POD).

Difference of Probabilities of Detection (dPOD): Difference of probabilities of detection is the difference between any two POD values. If the confidence interval of a dPOD does not contain zero, then the difference is statistically significant at the 5% level.

Microarray: A laboratory tool used to detect the expression of thousands of genes at the same time. DNA microarrays are 96-well plates that are printed as a matrix of oligonucleotide probe “Spots” in defined positions, with each spot containing a known DNA sequence.

### Materials and Methods

### Test Kit—PathogenDx QuantX Assay (Catalog Number.—QF-003 PathogenDx, LLC).

### Test Kit Components

a) QuantX Sample Preparation Kit
1) Lysis Buffer, 1 bottle (4 mL)
2) Neutralization Buffer, 1 vial (700 μL)
3) Sample Prep Buffer, 1 bottle (3.2 mL)
4) Sample Digestion Buffer, 1 vial (200 μL)
5) Promega RELIAPREP DNA Clean-up and Concentration System—100 reaction kit

b) PCR Master Mix
1) PCR Master Mix, 1 bottle (9 mL)
2) Primer Set 2: Quant Fungal, 1 vial (250 μL)
3) Standard, 1 vial (12 μL)
4) Taq polymerase, 1 vial (75 μL)

c) Hybridization and Analysis
1) Buffer 1, 1 bottle (7.5 mL)
2) Buffer 2, 1 bottle (3.5 mL)
3) QuantX Bacterial 96 well plate, 96 per 96-well plate
4) AUGURY software
   Key Equipment (Not part of the kit)

a) SENSOSPOT Fluorescence Microarray Analyzer (Sensospot Milteny Imaging GmbH, Germany)

b) MiniAmp Thermocycler, PN A37834 (ThermoFisher Scientific)

c) PCR Plate Spinner Centrifuge, PN C2000 (Light Labs)

### Sample Preparation

a) Cannabis flower (10 g). Mix 10 g of sample with 90 mL of PBS in a Whirl-Pak filter bag.

b) Perform a wash of the matrix by homogenizing for 10 sec.

c) Serially dilute the sample to the action level required for analysis (e.g. 1:1,000, 1:10,000, 1:100,000).

TABLE 17
Sample Buffer Mix volumes

| | Sample Prep Buffer | Sample Digestion Buffer |
|---|---|---|
| Number of Samples | (μL) | (μL) |
| 1 | 23.8 | 1.2 |
| 8 | 238 | 12 |
| 16 | 428.4 | 21.6 |
| 24 | 666.4 | 33.6 |
| 32 | 856.8 | 43.2 |
| 40 | 1047.2 | 52.8 |
| 48 | 1285.2 | 64.8 |
| 56 | 1475.6 | 74.4 |
| 64 | 1666 | 84 |
| 72 | 1856.4 | 93.6 |
| 88 | 2237.2 | 112.8 |
| 96 | 2427.6 | 122.4 |

### Analysis

a) Transfer 1 mL of the PBS suspension into a clean 1.5 mL conical tube, then centrifuge tube at 50×g for 3 minutes to pellet the excess matrix material.

b) Transfer the supernatant to a clean 1.5 mL tube, being careful to avoid matrix material. Discard the matrix pellet.

c) Centrifuge samples at 14,000×g for 3 minutes to pellet the cells.

d) Decant the supernatant and retain the cell pellet. Remove as much supernatant as possible without disturbing the pellet. It may be necessary to remove excess with a pipette.

e) Add 35 μL of Lysis Buffer to each tube, vortex to dislodge the pellet and quick spin.

f) Heat Sample tubes at 95+1° C. for 10 minutes.

g) Remove the tubes from the heat, vortex and briefly centrifuge.

h) To each tube add 5 μL of Neutralization Buffer and vortex thoroughly to mix.

i) Sample buffer Mix (make fresh each time) is prepared as shown in Table 17 by adding volumes of Sample Digestion Buffer and Sample Prep Buffer based on the number of samples being prepared.

j) Add 25 μL of Sample Buffer Mix to each tube, vortex to mix.

k) Heat sample tubes at 55+1° C. for 45 minutes.

l) Vortex for 10 seconds and briefly centrifuge samples to bring the fluid to bottom of the tube.

m) Heat sample tubes at 95+1° C. for 15 minutes.

n) Perform the Promega ReliaPrep DNA Clean-Up and Concentration System protocol using the following instructions:
1) Make sure the Column Wash Solution and Buffer B have had Molecular Biology Grade Ethanol (not provided with the kit) added to them following the instructions for the varying kit sizes.
2) Add 32.5 μL of Membrane Binding Solution to each prepped lysate and vortex for 5 seconds.
3) Add 97.5 μL of 100% isopropanol, not provided in the kit, to each prepped lysate, vortex to mix.
4) Load the sample onto the RELIAPREP Minicolumn seated in a collection tube and centrifuge for 30 seconds at 10,000×g.
5) Remove the column and discard the contents in the collection tube. Reseat the column into the collection tube.
6) Add 200 μL of Column Wash Solution (CWE) and centrifuge at 10,000×g for 15 seconds. Remove the column and discard the contents in the collection tube. Reseat the column into the collection tube.
7) Wash with 300 μL of Buffer B (BWB) and centrifuge at 10,000×g for 15 seconds. Repeat wash with 300 uL of Buffer B and centrifuge at 10,000×g again.
8) Remove column and discard the contents in the collection tube. Reseat the column into the same collection tube and centrifuge at 10,000×g for 1 minute to dry the column.
9) Transfer the column to a labelled Elution Tube.
10) Pipet 15 μL of Nuclease-Free water or TE Buffer, not provided, into the center of the RELIAPREP Minicolumn. The color should change from light to dark tan. Centrifuge at 10,000×g for 30 seconds.
11) For maximum recovery, repeat elution with an additional 15 μL of Nuclease Free Water or TE Buffer for a final volume of 30 μL.

c) Samples are now ready for PCR. Vortex and briefly centrifuge the tubes before removing 2 μL for PCR.

PCR amplification

a) Thaw PCR Master Mix and Primer Set.

b) Thaw the Standard tube on the Sample Prep Area bench top.
1) The High Standard is the stock tube.
2) The Low Standard is prepared by removing 5 μL of the vortexed High Standard tube to a new sterile tube.
3) Add 495 μL of Molecular Biology Grade Water, vortex to mix.
4) The Low Standard must be made fresh each time. Discard after use.
   Table 18 shows calculations for the appropriate volumes needed for the reaction. Labeling PCR Master Mix is made fresh each run.

TABLE 18
Labeling PCR Master Mix Volumes

| | | | Taq | Total |
|---|---|---|---|---|
| # of Reactions | PCR Master | Primer Set | Polymerase | Volume |
| per Primer | Mix (μL) | Fungal (μL) | (μL) | (μL) |
| 1 | 45.5 | 2 | 0.5 | 48 |
| 8 | 455 | 20 | 5 | 480 |
| 16 | 819 | 36 | 9 | 864 |
| 24 | 1183 | 52 | 13 | 1248 |
| 32 | 1638 | 72 | 18 | 1728 |
| 40 | 2002 | 88 | 22 | 2112 |
| 48 | 2366 | 104 | 26 | 2496 |
| 56 | 2730 | 120 | 30 | 2880 |
| 64 | 3185 | 140 | 35 | 3360 |
| 72 | 3549 | 156 | 39 | 3744 |
| 80 | 3913 | 172 | 43 | 4128 |
| 88 | 4277 | 188 | 47 | 4512 |
| 96 | 4641 | 204 | 51 | 4896 |

(a) Vortex all reagents except the Taq Polymerase for 15 seconds; centrifuge at 1000×g speed for 3-5 seconds.
(b) Mix the indicated reagent volumes (calculated from Table 18) in a microfuge tube to prepare PCR Master Mix.
(c) Briefly vortex PCR Master Mix and centrifuge at 1000×g for 3-5 seconds.
(d) Store all reagents at −20° C. after use.
(e) Pipette 48 μL of Labeling PCR Master Mix into the bottom of PCR tubes or wells of a PCR plate.
(f) In the Sample Prep area, pipette 2 μL of sample lysate, 2 μL of the High Standard and 2 μL of the Low Standard into the bottom of the corresponding tube or well for a final volume of 50 μL per PCR reaction. Pipette up and down to mix.
(g) Cap tubes, or seal plates with PCR film ensuring every well is completely sealed.
(h) Centrifuge at 1000×g for 3-5 second.
(i) Move to the Hybridization Area/Post PCR Area. Place tubes or plate into the thermal cycler with a pressure pad if necessary, before closing the thermal cycler lid.
(j) Enter the PCR Program into your thermal cycler as shown in Table 19. Confirm all parameters.
(k) Once the PCR is complete, the plate may be stored at 4° C. for up to 1 weeks.

TABLE 19
Labeling PCR Program

| Steps | Temp. | Time | | Cycles |
|---|---|---|---|---|
| 1 | 95° C. | 4 | Minutes | 1 |
| 2 | 95° C. | 30 | seconds | 40 |
| 3 | 55° C. | 30 | seconds | |
| 4 | 72° C. | 1 | minute | |
| 5 | 72° C. | 7 | minutes | 1 |

| | | | |
|---|---|---|---|
| 6 | 15° C. | ∞ | 1 |

### Hybridization

a) Perform all steps in the Hybridization/Post PCR Area.

b) Before starting, thaw Buffer 2 at room temperature.
1) Place the plate to be used in the Hybridization Chamber.
2) Ensure the wells to be used have been clearly tracked.
3) Carefully remove the foil seal from only the wells that will be hybridized.
   Use a clean razor blade or other precision blade to carefully cut the seal between the wells to be used and the wells that should remain covered for future use. Gently peel the seal from the wells you are going to use.
4) Leave the remainder of the wells covered to avoid any contact with moisture.

c) Prepare the Pre-hybridization Buffer and Hybridization Buffers in sterile tubes for the number of wells that will be hybridized as per Tables 20 and 21. The tables shown below have the volumes required to make one well. Multiply the reagent volumes by the number of wells to be run. Add extra wells to account for pipetting loss. Vortex briefly to mix.

d) Apply 200 μL of Molecular Biology Grade Water to each well while being careful to avoid contact with the array.

e) Aspirate and then again, dispense 200 μL of Molecular Biology Grade Water to each well and allow to sit covered in the Hybridization Chamber for 5 minutes before aspirating water from the plate.

f) Aspirate the water wash and add 200 μL of Pre-hybridization Buffer to each designated well of the PathogenDx plate without touching the pipette tip to the array surface. Close the Hybridization Chamber box lid.

g) Allow Pre-hybridization Buffer to stay on the arrays for 5 minutes; do not remove the plate from the Hybridization Chamber.

h) Briefly centrifuge the tubes or plate containing the Labeling PCR product.

i) Add 18 μL of Hybridization Buffer to each well of the Labeling PCR product for hybridization within the 96-well PCR plate or tubes, pipette up and down to mix. It is important that no cross-contamination occurs during this step. The PCR product and the Hybridization Buffer mix constitute the Hybridization Cocktail.

j) Aspirate the Pre-hybridization Cocktail from the arrays.

k) Immediately add 68 μL of the Hybridization Cocktail to each array being careful not to touch the array surface with the pipette tip. Ensure that the sample ID and location are recorded.

l) Close the Hybridization Chamber lid.

m) Allow to hybridize for 30 minutes at room temperature in the Hybridization Chamber.

TABLE 20
Reagent volumes for preparation of Pre-hybridization Buffer

| | Volumes corresponding to the number of wells | | | | | | | | | | | | |
|---|---|---|---|---|---|---|---|---|---|---|---|---|---|
| Pre-hybridization | 1 | 8 | 16 | 24 | 32 | 40 | 48 | 56 | 64 | 72 | 80 | 88 | 96 |
| reagents | well | wells | wells | wells | wells | wells | wells | wells | wells | wells | wells | wells | wells |
| Molecular biology | 137.6 | 1651 | 2752 | 3853 | 5229 | 6330 | 7430 | 8531 | 9907 | 11008 | 12109 | 13210 | 14310 |
| grade water (μL) | | | | | | | | | | | | | |
| Buffer 1 (μL) | 40.9 | 490.8 | 818 | 1145 | 1554 | 1881 | 2209 | 2536 | 2945 | 3272 | 3599 | 3926 | 4254 |
| Buffer 2 (μL) | 21.5 | 258 | 430 | 602 | 817 | 989 | 1161 | 1333 | 1548 | 1720 | 1892 | 2064 | 2236 |

TABLE 21
Reagent volumes for preparation of Hybridization Buffer

| | Volumes corresponding to the number of wells | | | | | | | | | | | | |
|---|---|---|---|---|---|---|---|---|---|---|---|---|---|
| Hybridization | 1 | 8 | 16 | 24 | 32 | 40 | 48 | 56 | 64 | 72 | 80 | 88 | 96 |
| reagents | well | wells | wells | wells | wells | wells | wells | wells | wells | wells | wells | wells | wells |
| Buffer 1 (μL) | 11.8 | 141.6 | 236 | 330.4 | 448.4 | 542.8 | 637.2 | 731.6 | 849.6 | 944 | 1038 | 1133 | 1227 |
| Buffer 2 (μL) | 6.2 | 74.4 | 124 | 173.6 | 235.6 | 285.2 | 334.8 | 384.4 | 446.4 | 496 | 545.6 | 595.2 | 644.8 |

TABLE 22
Reagent volumes for preparation of Wash Buffer

| | Volumes corresponding to the number of wells | | | | | | | | | | | | |
|---|---|---|---|---|---|---|---|---|---|---|---|---|---|
| Wash Buffer | 1 | 8 | 16 | 24 | 32 | 40 | 48 | 56 | 64 | 72 | 80 | 88 | 96 |
| reagents | well | wells | wells | wells | wells | wells | wells | wells | wells | wells | wells | wells | wells |
| Buffer 1 (μL) | 4.5 | 54 | 90 | 126 | 171 | 207 | 243 | 279 | 324 | 360 | 396 | 432 | 468 |
| Molecular biology | 0.5955 | 6.714 | 11.19 | 15.666 | 21.261 | 25.737 | 30.213 | 34.689 | 40.284 | 44.76 | 49.236 | 53.712 | 58.188 |
| grade water (μL) | | | | | | | | | | | | | |

Post hybridization PathogenDx slide processing

a) Prepare Wash buffer according to the number of wells to be used (Table 22). Washing must be performed according to the protocol to ensure detectable signal and adequate washing to prevent elevated background signals.

b) Aspirate the Hybridization Cocktail from the slides.

c) Add 200 μL of Wash Buffer to each array, then aspirate.

d) Add 200 μL of Wash Buffer a second time to each array, close the Hybridization Chamber lid and allow buffer to remain on the slides for 10 minutes.

e) Aspirate the Wash Buffer.

f) Perform a final wash by dispensing and aspirating 200 μL of Wash Buffer, aspirate immediately.

g) Following the last aspiration step, remove the slides from the Hybridization Chamber.

h) Dry the plate using the plate centrifuge for 1 minute.
1) Place the plate face down with the open wells against paper towels to absorb liquid during centrifugation.
2) After 1 minute, remove the plate and inspect for any remaining moisture. If moisture is present, repeat the centrifugation step until completely dry.

i) Prior to scanning, clean the back of the glass microarray with lens paper or Kim wipe (never use paper towels which leave an excess of fibers and interferes with scanning).
1) If the back of the slide still shows dust and/or streaks, lightly spray the back of the plate with 70% ethanol and wipe dry.

j) PathogenDx plates should be placed back into a moisture barrier bag with desiccant until scanning may be performed in order to protect the arrays from light. Plates should be scanned within two weeks of hybridization.

Scanning conditions and Data Acquisition

a) Access the Sensovation scanner desktop, select the application “Array Reader”.

b) Open the tray, select “Open Tray”.

c) Place the microarray into the tray oriented with the plate face up and aligned with A1 in the top left corner.

d) Close the tray, select “Close Tray”.

e) Select “Scan”.

f) From the dropdown menu for “Rack Layout” select the Full Slide (96 wells) PDx.

g) From the dropdown menu for assay layout, select “PathogenDx Assay 002”.

h) Click on the three dots icon to the right of “Scan Position”.

i) To scan a full plate, double click the asterisk at the top left of the plate image.

j) To scan a partial plate, click the desired wells or click on the column number.

k) Select the Blue Arrow to begin the scanning process.

l) While the plate is being scanned, select “Result overview” to review the images of the wells.

m) When the plate is finished scanning and the screen displays the digital image of a plate with all green wells, select the Red X to exit the scanning process.

n) Open the tray, select “Open Tray”.

o) Remove the microarray and store inside the moisture barrier bag with the desiccant packets.

p) Close the tray, select “Close Tray”.

q) Exit the Array Reader application, select “Exit”.

r) On the Sensovation Scanner desktop, select the folder “Scan Results”.

s) Locate the folder associated with your plate and rename the folder with the plate barcode number y scanning the barcode located either on the outside of the barrier bag or on the plate itself.
1) If a full plate was scanned, rename the scan file to reflect the plate barcode. For example, rename “ScanJob-191108130334_1” to “7024001001”.
2) If a partial plate was scanned, add the wells scanned to the end of the barcode. For example, if the first two columns were scanned rename“ScanJob-191108130334_1” to “7024001001.well001-well016”.

t) Submit the whole barcode labeled folder to Portal.

u) Refer to the Portal instructions for Analysis.

### Interpretation and Test Results Report

a) Data is analyzed automatically by the software.

b) Table 23 was used to determine the final interpretation.

### Confirmation

For samples that fail an action limit, confirm by streaking the test aliquot onto Dichloran Rose Bengal Chloramphenicol (DRBC) agar. DRBC plates should be incubated for 5-7 days at 25±1° C. Growth on the plate is confirmation that the sample is positive at that action limit level.

TABLE 23
Interpretation of Results

| | TOTAL YEAST and MOLD | |
|---|---|---|
| Action Limit Evaluated | Result (CFU/g) | Interpretation |
| 1:1,000 | <1,000 | Pass |
| | >1,000 | Fail |
| 1:10,000 | <10,000 | Pass |
| | >10,000 | Fail |
| 1:100,000 | <100,000 | Pass |
| | >100,000 | Fail |

### Example 5

### AOAC Validation Study

### Study Overview

This validation study was conducted under the AOAC Research Institute Performance Tested Method (PTM) ERV program and the AOAC INTERNATIONAL Methods Committee Guidelines for Validation of Microbiological Methods for Food and Environmental Surfaces (6). The QuantX method was compared to plating on DRBC for the detection of total viable yeast and mold in cannabis flower at specific dilution thresholds. Inclusivity and exclusivity was also performed. The matrix study was performed by an independent laboratory, SV Laboratories (Kalamazoo, Mich.). The inclusivity and exclusivity analysis was performed by Q Laboratories (Cincinnati, Ohio).

### Inclusivity/Exclusivity

Inclusivity Methodology. Inclusivity and exclusivity strains were evaluated to meet the requirements of the AOAC ERV PTM study protocol. For the ERV study, 50 strains of yeast and mold, and 30 exclusivity strains were evaluated. We are currently in the process of evaluating the remaining exclusive strains. Target strains were cultured in potato dextrose broth or on potato dextrose agar until appropriate growth was observed. After incubation, cultures were diluted in PBS to levels of 100-1000 CFU/mL. Exclusivity strains were cultured onto non-selective agar under optimal conditions for growth and tested undiluted.

A 1.0 mL aliquot from the diluted target or undiluted non-target culture were randomized, blind coded and analyzed by the QuantX method.

### Results

Of the additional inclusivity strains tested, all were correctly detected. All exclusivity cultures were non-detected. Tables 24 and 25 presents a summary of the results.

TABLE 24
Results for Inclusivity of the QuantX Method

| No. | Organism | QuantX Result |
|---|---|---|
| 1 | Kluyveromyces lactis | Pass |
| 2 | Saccharomyces kudriavzevii | Pass |
| 3 | Zygosaccharomyces bailii | Pass |
| 4 | Kloeckera species | Pass |
| 5 | Candida albicans | Pass |
| 6 | Candida lusitaniae | Pass |
| 7 | Candida tropicalis | Pass |
| 8 | Dekkera bruxellensis | Pass |
| 9 | Aureobasidium pullulans | Pass |
| 10 | Rhodotorula mucilaginosa | Pass |
| 11 | Cryptococcus neoformans | Pass |
| 12 | Debaromyces hansenii | Pass |
| 13 | Purpureocillium lilacinum | Pass |
| 14 | Yarrowia lipolytica | Pass |
| 15 | Wickerhamomyces anomala | Pass |
| 16 | Stemphylium species | Pass |
| 17 | Penicillium venetum | Pass |
| 18 | Paecilomyces marquandii | Pass |
| 19 | Scopulariopsis acremonium | Pass |
| 20 | Mucor hiemalis | Pass |
| 21 | Mucor circinelloides | Pass |
| 22 | Talaromyces pinophilus | Pass |
| 23 | Aspergillus fumigatus | Pass |
| 24 | Talaromyces flavus | Pass |
| 25 | Rhizopus stolonifera | Pass |
| 26 | Cladosporium halotolerans | Pass |
| 27 | Rhizopus oryzae | Pass |
| 28 | Cladosporium herbarum | Pass |
| 29 | Aspergillus aculeatus | Pass |
| 30 | Penicillium chrysogenum | Pass |
| 31 | Chaetomium globosum | Pass |
| 32 | Arthrinium aureum | Pass |
| 33 | Aspergillus brasilliensis | Pass |
| 34 | Aspergillus caesiellus | Pass |
| 35 | Curvularia lunata | Pass |
| 36 | Cryptococcus laurentii | Pass |
| 37 | Aspergillus terreus | Pass |
| 38 | Byssochlamys fulva | Pass |
| 39 | Penicillium rubens | Pass |
| 40 | Geotrichum candidum | Pass |
| 41 | Aspergillus flavus | Pass |
| 42 | Fusarium solani | Pass |
| 43 | Botrytis cinerea | Pass |
| 44 | Aspergillus niger | Pass |
| 45 | Aspergillus oryzae | Pass |
| 46 | Fusarium proliferatum | Pass |
| 47 | Fusarium oxysporum | Pass |
| 48 | Paecilomyces variotii | Pass |
| 49 | Geotrichum silvicola | Pass |
| 50 | Alternaria alternata | Pass |

TABLE 25
Results for Exclusivity of the QuantX Method

| No. | Organism | QuantX Result |
|---|---|---|
| 1 | Acinetobacter baumanii | Pass |
| 2 | Aeromonas hydrophila | Pass |
| 3 | Burkholderia cepacia | Pass |
| 4 | Citrobacter braakii | Pass |
| 5 | Citrobacter farmeri | Pass |
| 6 | Edwardsiella tarda | Pass |
| 7 | Enterobacter cloacae | Pass |
| 8 | Escherichia coli | Pass |
| 9 | Hafnia alvei | Pass |
| 10 | Listeria monocytogenes | Pass |
| 11 | Pantoea agglomerans | Pass |
| 12 | Proteus mirabilis | Pass |
| 13 | Pseudomonas aeruginosa | Pass |
| 14 | Pseudomonas gessardii | Pass |
| 15 | Rahnella aquatilis | Pass |
| 16 | Stenotrophomonas maltophilia | Pass |

### Matrix Studies—Methodology

Cannabis test portions were prepared from Steadfast Analytical Laboratory's inventory of retained samples from its Michigan-licensed grower, patient, and caregiver customers. The samples were screened for yeast and mold prior to the study, using a rapid automated enumeration method in order to prepare matrix batches at the target contamination levels of <1000, ˜1000, ˜10000, and ˜100000 CFU/g.

Using sterilized aluminum containers, individual samples that produced results within a specified contamination level were combined to produce four batches (control, low, medium and high). Batches were manually mixed in an aseptic manner until homogeneous.

For each contamination level, five replicates were quantified by spread plating aliquots of the samples onto DRBC agar. Plating results indicated that yeast and mold levels for the control, low, medium, and high batches prepared for analysis were 350, 890, 13000, and 100000 CFU/g, respectively.

Five replicate test portions at the control and high levels, and 20 replicate test portions at the low and medium levels, were tested. A fractional positive data set (25-75% of test portions positive) was required for at least one of the intermediate levels at a minimum of one test threshold. Individual 10 g test portions from each contamination level were prepared in sterile filter Whirl-Pak bags. Test portions were assigned identification tags following Michigan's Marijuana Regulatory Agency (MRA) seed-to-sale system for distribution and tracking, including blind coding the contamination level of the test portions. The individual samples were also assigned random sample numbers for reporting results to the AOAC Research Institute. A technician at the independent laboratory not involved in the coding process performed the analyses.

Each test portion was combined with 90 mL PBS. Test portions were homogenized by hand and further 1:100, 1:1000 and 1:10,000 dilutions prepared using PBS as the diluent. From the final 1:1000 and 1:10000 dilutions, 1 mL aliquots were analyzed by the QuantX method.

For confirmation, 10 μL aliquots of the dilutions evaluated were streaked to DRBC agar. Plates were incubated at 25±1° C. for 5-7 days after which they were examined for yeast or mold growth.

### Results

As per criteria outlined in Appendix J of the Official Methods of Analysis Manual and specified in the study protocol, fractional positive results were obtained for one of the dilution levels evaluated. Fractional positive data sets were obtained for the low level at the >1000 CFU/g test threshold. At this threshold, all control-level test portions produced negative results and all high-level test portions produced positive results.

Of the 100 data points encompassing all levels and test thresholds, there were seven instances of disagreement between presumptive and confirmed results: three low-level test portions at the >1000 CFU/g threshold were presumptive positive/confirmed negative, one medium-level test portion at the >1000 CFU/g threshold was presumptive positive/confirmed negative, one medium-level test portions at the >1000 CFU/g threshold were presumptive negative confirmed positive, and two high-level test portion at the >10000 CFU/g threshold was presumptive negative/confirmed positive.

The probability of detection (POD) was calculated for the candidate presumptive results, POD_{CP} and the candidate confirmed results, POD_{CC}, as well as the difference in the presumptive and confirmed results, dPOD_{CP}. The POD analysis between the QuantX assay presumptive and confirmed results indicated that there was not a statistically significant difference. A summary of POD analyses are presented in Table 26.

### Discussion

In the matrix study, the QuantX⁻Fungal assay successfully detected the target analyte from cannabis flower samples. The QuantX method demonstrated a high level of specificity in detecting the 50 inclusive organisms and no detection of the 30 exclusive organisms (Table 8 and 9). The POD statistical analysis in Table 10, indicated that the candidate method performance was identical to the reference method at low levels (320 CFU/g) but at the 890 CFU/g was statistically different than the reference method (95% CI −0.05, 0.35) with the candidate method detecting more positive samples. The two methods performed identical at the 13,000 CFU/g, both detecting 90% of the samples at the >1000 threshold and 0% at the >10,000 threshold. While it should be noted that the samples used in this study were held longer for analysis and may have resulted in the lower detection at the high level, the results of the QuantX and DRBC plating method align closely.

Thus, data from this study supports the product claim that the QuantX assay can detect total yeast and mold from cannabis flower at specific action thresholds used by state regulatory agencies. Data from the inclusivity and exclusivity analysis indicates the method is highly specific and can detect a wide range of target organisms and discriminate them from background organisms and near neighbors. The results obtained by the POD analysis of the method comparison study demonstrated that the candidate methods performance was not statistically different than that of the culture confirmation method.

TABLE 26
QuantX TYM presumptive and confirmed results fortesting of dried cannabis
flower. Comparison between QuantX assay and plating (MH/PU).

| | | | Test | | | | | | | | | |
|---|---|---|---|---|---|---|---|---|---|---|---|---|
| | | Level | Threshold | | QuantX TYM Presumptive | | | Quant TYM Confirmed | | | | |
| Matrix | Strain | (CFU/g)^{a} | (CFU/g)^{b} | N^{c} | x^{d} | POD_{CP}^{e} | 95% CI | x | POD_{CC}^{f} | 95% CI | dPOD_{CP}^{g} | 95% CI^{h} |
| Dried | Naturally | 320 | >1000 | 5 | 0 | 0 | 0.00, | 0 | 0 | 0.00, | 0.00 | −0.47, |
| Cannabis | Contaminated | | | | | | 0.43 | | | 0.43 | | 0.47 |
| Flower | | | >10000 | 5 | 0 | 0 | 0.00, | 0 | 0 | 0.00, | 0.00 | −0.47, |
| | | | | | | | 0.43 | | | 0.43 | | 0.47 |
| | | 890 | >1000 | 20 | 9 | 0.45 | 0.26, | 6 | 0.30 | 0.14, | 0.15 | −0.05, |
| | | | | | | | 0.66 | | | 0.52 | | 0.35 |
| | | | >10000 | 20 | 0 | 0.00 | 0.00, | 0 | 0.00 | 0.00, | 0.00 | −0.13, |
| | | | | | | | 0.16 | | | 0.16 | | 0.13 |
| | | 13000 | >1000 | 20 | 18 | 0.90 | 0.70, | 18 | 0.90 | 0.70, | 0.00 | −0.19, |
| | | | | | | | 0.97 | | | 0.97 | | 0.19 |
| | | | >10000 | 20 | 0 | 0.00 | 0.00, | 0 | 0.00 | 0.00, | −0.05 | −0.13, |
| | | | | | | | 0.16 | | | 0.16 | | 0.13 |
| | | 100000 | >1000 | 5 | 5 | 1 | 0.57, | 5 | 1 | 0.57, | 0.00 | −0.47, |
| | | | | | | | 1.00 | | | 1.00 | | 0.47 |
| | | | >10000 | 5 | 0 | 1 | 0.00, | 2 | 0.40 | 0.12, | −0.40 | −1.00, |
| | | | | | | | 0.43 | | | 0.77 | | 0.21 |

^{a}From aerobic viable yeast and mold plate count (DRBC).
^{b}Based on dilution and volume of sample tested. A positive result indicates contamination above the test threshold level.
^{c}N = Number of test portions.
^{d}x = Number of positive test portions.
^{e}POD_{CP} = Candidate method presumptive positive outcomes divided by the total number of trials.
^{f}POD_{CC} = Candidate method confirmed positive outcomes divided by the total number of trials.
^{g}dPOD_{CP} = Difference between the candidate method presumptive result and candidate method confirmed result POD values.
^{h}95% CI = If the confidence interval of a dPOD does not contain zero, then the difference is statistically significant at the 5% level.

### Example 6

Detection of Fungus in a plant sample

The method described below shows the developed trendline used for mathematical modeling modifications to the Augury Software (Augury Technology, NY).

### Materials & Methods

### Extraction of Fungal Nucleic Acids

1 mL aliquots of A. nidulans (10\hat{ }5-10\hat{ }2) is transferred into a clean 1.5 mL tube and centrifuged (14,000×g for 3 minutes). The resulting supernatant from this step is decanted and the cell pellet retained. Lysis buffer (35 μl) is added to each tube, vortexed and heated at 95° C. for 10 min. The samples are removed from the heat source and centrifuged (2000×g for 5 seconds). To each tube, 5 μl of neutralization buffer is added and vortexed thoroughly to mix. Sample Buffer Mix (Table 17) is prepared and 25 μl added to each tube and vortexed to mix. The sample tubes are heated at 55° C. for 45 min to allow complete sample digestion. The samples are removed from the heat source and vortexed for 10 s. The sample tubes are then heated at 95° C. for 15 min.

Sample cleanup using RELIAPREP Kit

To each prepped lysate was added 32.5 μl of membrane binding solution and vortexed for 5 s. Isopropanol (97.54 of 100%) was added and vortexed for another 5 s. The sample was then loaded onto a RELIAPREP mini column seated in a collection tube, and centrifuged (10,000×g, 30 s). The contents in the collection tube were discarded, the column reseated into the collection tube and bound sample washed with 200 μL of Column Wash Solution (centrifuge at 10,000×g, 15 s). The contents were discarded, and the bound sample washed with 300 μL of Buffer B (centrifuge at 10,000×g, 15 s), repeating the wash one more with 300 μL of Buffer B. The contents were discarded, and the column centrifuged for 1 min to dry the column. The column was then transferred to a labelled Elution Tube, 154 of Nuclease-Free water or TE Buffer added and centrifuged for 30 s. Elution was repeated with an additional 154 of Nuclease Free Water or TE Buffer to maximize recovery.

Labeling PCR amplification

Reagents (PCR Master Mix, Primer Set, and High Standard) were thawed. The Low Standard was prepared by mixing 5 μl of the vortexed High Standard tube with 495 μl of Molecular Biology Grade Water and vortexed to mix. Table 18 was used as reference to calculate the appropriate reagent volumes needed based on the number of samples. All reagents (except Taq polymerase) were vortexed for 15 s and centrifuged (1000×g for 5 s). The indicated reagent volumes were mixed in a microfuge tube to prepare the Labeling PCR Master Mix. The PCR master mix was briefly vortexed and centrifuged (1000×g for 5 s). Amplification conditions were as shown in Table 19. The following primers were used—Forward primer SEQ ID NO:133, final concentration 50 nM) and Reverse primer (SEQ ID NO:134, 5′Cy3 labeled, final concentration 200 nM).

### Hybridize PCR Amplified Product to Microarray

The Pre-hybridization Buffer and Hybridization Buffers were prepared in sterile tubes for the number of wells that will be hybridized (Tables 27 and 28) and vortexed to mix. The plate was placed in the Hybridization Chamber and the foil seal carefully removes from the wells to be hybridized. Molecular Biology Grade water (200 μL) was applied to each well, aspirated and another 200 μL of Molecular Biology Grade water added to each well. The plate was incubated in the Hybridization Chamber for 5 min and the water aspirated. Pre-hybridization Buffer (200 μL) was added to each designated well and allowed to sit covered in the Hybridization Chamber for 5 min. Hybridization Buffer (18 μL) was added to each well for hybridization within the 96-well PCR plate and pipetted up and down to mix. The Pre-hybridization Cocktail was aspirated from the array and the Hybridization Cocktail (68 μL) added immediately to each array. The plate was allowed to hybridize for 30 min at room temperature in the Hybridization Chamber. Wash Buffer was prepared (Table 29) and vortexed briefly to mix prior to adding (200 μl) to each array followed by aspirating immediately. Another 200 μL of Wash Buffer was added and incubated for 10 min. A final wash was performed by dispensing 200 μL of Wash Buffer and aspirating immediately. The plate was dried using a plate centrifuge for 5 min.

TABLE 27
Pre-hybridization buffer volumes

| Pre-hybridization reagents | Volumes corresponding to one well | |
|---|---|---|
| Molecular Biology Grade water | 137.6 | μL |
| Buffer 1 | 40.9 | μL |
| Buffer 2 | 21.5 | μL |

TABLE 28
Hybridization buffer volumes

| Hybridization reagents | Volumes corresponding to one well |
|---|---|
| Buffer 1 | 40.9 μL |
| Buffer 2 | 21.5 μL |

TABLE 29
Wash buffer volumes

| Wash buffer reagents | Volumes corresponding to one well | |
|---|---|---|
| Buffer 1 | 5 | μL |
| Molecular Biology Grade water | 555 | μL |

### Results

A. nidulans cells prepared at 10⁵ down to 10² dilutions were run to establish a trendline for Augury software calculations. The high, medium, and low Total Yeast and Mold RFU values correspond to the CFU values in the cell curve data.

### Discussion

As the cannabis industry enters an era of acceptance at a national level, the methods developed by PathogenDx as disclosed in this invention are of direct relevance to cannabis testing at the national level. The suite of advanced testing and reporting technologies raises cannabis testing closer to the level of efficacy and standardization required of labs in other mainstream industries.

The one-step PCR for its QuantX fungal assay method described in this invention employs sample preparation step using RELIAPREP (Promega Corporation, WI). RELIAPREP shortens the assay process by consolidating the two-step PCR into a single PCR step, enabling results to be delivered in 4.5 hours instead of 6 hours, and helps concentrate the sample for improved sensitivity. Overall, the new methodology for preparing and analyzing cannabis improves assay reliability by reducing PCR inhibition and minimizing all types of dim signal.

Implementation of the expanded 96-well microarray format introduces to the cannabis industry a best practice commonly used in clinical labs. Instrumentation, reagents, and consumables are naturally fitted to a 96-well plate format for a higher level of efficiency, throughput, leading to economical scaling compared to prior 12-well formats. The methods described in this invention are supported by other improvements including, the industry-first foil-sealed wells that enable lab technicians to uncover only the wells needed to test samples received on that day or shift, thereby realizing significant cost savings from reduced waste of unused wells and test media. Moreover, the expanded microarray is made with higher quality glass that provides improved performance for both specificity and imaging accuracy.

To provide another level of granularity in test results reporting, PathogenDx is migrating from Dropbox to a custom PathogenDx Reporting Portal for cannabis compliance reporting. PathogenDx's intuitive, user-friendly portal drives customer ease and efficiency by reducing the number of steps necessary to obtain lab results and COAs. This also improves data visibility with multi-user access to real-time results tracking and prior history reports.

While the foregoing written description of an embodiments enables one of ordinary skill to make and use what is considered presently to be the best mode thereof, those of ordinary skill will understand and appreciate the existence of variations, combinations, and equivalents of the specific embodiment, method, and examples herein. The present disclosure should therefore not be limited by the above-described embodiments, methods, and examples, but by all embodiments and methods within the scope and spirit of the present disclosure.

The following references are cited herein.
1. Emerald Scientific Cannabis Testing Regulations by State: Increase Your Knowledge. Emerald scientific.com/blog/cannabis-testing-regulations-by-state-increase-your-knowledge/(2018).
2. Verweij, et al. JAMA 284:2875 (2000).
3. Thompson, et al. Clinical Microbiology and Infection. 23(4):269-70 (2017).
4. Kern, R. and Green, J. R. Cannabis Science and Technology, November/December 2:(6) (2019). Cannabissciencetech.com/view/its-not-too-late-post-harvest-solutions-microbial-contamination-issues.
5. Colorado Department of Revenue Enforcement Division-Marijuana. MED 2019 Annual Update (2019). Drive.google.com/file/d/1rCWw9AquV9Pr1STMbv8dySrU6L2wJHfl/view.
6. Official Methods of Analysis 21st Ed., Appendix J: AOAC INTERNATIONAL, Rockville, Md., (2019). Eoma.aoac.org/app_j.pdf.

What is claimed is:

1. A method for quantitating a fungus on a plant, comprising:
a) obtaining a sample from the plant;
b) isolating from the sample, total nucleic acids;
c) performing on the total nucleic acids an asymmetric PCR amplification reaction using at least one fluorescent labeled primer pair comprising an unlabeled primer, and a fluorescently labeled primer, selective for a target nucleotide sequence in the fungus to generate at least one fluorescent labeled fungal amplicon;
d) hybridizing the fluorescent labeled fungal amplicons to a plurality of nucleic acid probes each having a sequence corresponding to a sequence determinant in the fungus, each of said nucleic acid probes attached at a specific position on a solid microarray support;
e) washing the microarray at least once;
f) imaging the microarray to detect at least one fluorescent signal from the hybridized fluorescent labeled fungal amplicons; and
g) calculating an intensity of the fluorescent signal, said intensity correlating with a quantity of the fungus in the sample, thereby quantitating the fungus on the plant.

2. The method of claim 1, further comprising isolating a total DNA after step b, said step c comprising performing the asymmetric PCR amplification reaction on the total DNA.

3. The method of claim 1, wherein the fluorescently labeled primer is in an excess of about 4-fold to about 8-fold over the unlabeled primer in the fluorescent labeled primer pair.

4. The method of claim 1, wherein the fungus is a yeast or a mold.

5. The method of claim 4, wherein the fungus is an Aspergillus species.

6. The method of claim 1, wherein the unlabeled primer is a forward primer comprising the nucleotide sequences of SEQ ID: 13, SEQ ID: 15, SEQ ID: 31, SEQ ID: 33, SEQ ID: 133, or SEQ ID: 135.

7. The method of claim 1, wherein the fluorescently labeled primer is a reverse primer comprising the nucleotide sequences of SEQ ID: 14, SEQ ID: 16, SEQ ID: 32, SEQ ID: 34, or SEQ ID: 134.

8. The method of claim 1, wherein the nucleic acid probes have at least one probe nucleotide sequence selected from the group consisting of SEQ ID NOS: 86-126 and 136-140.

9. The method of claim 1, wherein the plant is a cannabis or a hemp, or a product derived therefrom.

10. The method of claim 9, wherein the product is an oil.

11. A method for quantitating at least one fungus in an agricultural product, comprising:
a) obtaining a sample of the agricultural product;
b) isolating total nucleic acids from the sample;
c) performing on the total nucleic acids an asymmetric PCR amplification reaction using at least one fluorescent labeled primer pair comprising an unlabeled primer, and a fluorescently labeled primer, selective for a target nucleotide sequence in the fungus to generate at least one fluorescent labeled fungal amplicon;
d) hybridizing the fluorescent labeled fungal amplicons to a plurality of nucleic acid probes each having a sequence corresponding to a sequence determinant in the fungus, each of said nucleic acid probes attached at a specific position on a solid microarray support;
e) washing the microarray at least once;
f) imaging the microarray to detect at least one fluorescent signal from the hybridized fluorescent labeled fungal amplicons; and
g) calculating an intensity of the fluorescent signal, said intensity correlating with a quantity of the fungus in the sample, thereby quantitating the at least one fungus in the agricultural product.

12. The method of claim 11, further comprising isolating a total DNA after step b, said step c comprising performing the asymmetric PCR amplification reaction on the total DNA.

13. The method of claim 11, wherein the fluorescently labeled primer is in an excess of about 4-fold to about 8-fold over the unlabeled primer in the fluorescent labeled primer pair.

14. The method of claim 11, wherein the fungus is a yeast or a mold.

15. The method of claim 14, wherein the fungus is an Aspergillus species.

16. The method of claim 11, wherein the unlabeled primer is a forward primer comprising the nucleotide sequences of SEQ ID: 13, SEQ ID: 15, SEQ ID: 31, SEQ ID: 33, SEQ ID: 133, or SEQ ID: 135.

17. The method of claim 11, wherein the fluorescently labeled primer is a reverse primer comprising the nucleotide sequences of SEQ ID: 14, SEQ ID: 16, SEQ ID: 32, SEQ ID: 34, or SEQ ID: 134.

18. The method of claim 11, wherein the nucleic acid probes have at least one probe nucleotide sequence selected from the group consisting of SEQ ID NOS: 86-126 and 136-140.

19. The method of claim 11, wherein the agricultural product is obtained from a cannabis, or a hemp.

20. The method of claim 11, wherein the agricultural product is an oil.


SEQUENCE LISTING (140 sequences)

SEQ ID NO: 1 - DNA, 23 bp, Artificial Sequence
  Forward primer for amplification of the 16S rDNA HV3 locus in all bacteria
tttcacaytggractgagacacg

SEQ ID NO: 2 - DNA, 27 bp, Artificial Sequence
  Reverse primer for amplification of the 16S rDNA HV3 locus in all bacteria
tttgactaccagggtatctaatcctgt

SEQ ID NO: 3 - DNA, 28 bp, Artificial Sequence
  Forward primer for amplification of the Stx1 locus in pathogenic Escherichia coli
tttataatctacggcttattgttgaacg

SEQ ID NO: 4 - DNA, 29 bp, Artificial Sequence
  Reverse primer for amplification of the Stx1 locus in pathogenic Escherichia coli
tttggtatagctactgtcaccagacaatg

SEQ ID NO: 5 - DNA, 26 bp, Artificial Sequence
  Forward primer for amplification of the Stx2 locus in pathogenic Escherichia coli
tttgatgcatccagagcagttctgcg

SEQ ID NO: 6 - DNA, 27 bp, Artificial Sequence
  Reverse primer for amplification of the Stx2 locus in pathogenic Escherichia coli
tttgtgaggtccacgtctcccggcgtc

SEQ ID NO: 7 - DNA, 28 bp, Artificial Sequence
  Forward primer for amplification of the InvA locus in all Salmonella
tttattatcgccacgttcgggcaattcg

SEQ ID NO: 8 - DNA, 28 bp, Artificial Sequence
  Reverse primer for amplification of the InvA locus in all Salmonella
tttcttcatcgcaccgtcaaaggaaccg

SEQ ID NO: 9 - DNA, 26 bp, Artificial Sequence
  Forward primer for amplification of the tuf locus in all Escherichia coli
tttcagagtgggaagcgaaaatcctg

SEQ ID NO: 10 - DNA, 26 bp, Artificial Sequence
  Reverse primer for amplification of the tuf locus in all Escherichia coli
tttacgccagtacaggtagacttctg

SEQ ID NO: 11 - DNA, 30 bp, Artificial Sequence
  Forward primer for amplification of the 16S rDNA HV3 locus in Enterobacteriaceae
ttaccttcgggcctcttgccatcrgatgtg

SEQ ID NO: 12 - DNA, 33 bp, Artificial Sequence
  Reverse primer for amplification of the 16S rDNA HV3 locus in Enterobacteriaceae
ttggaattctacccccctctacragactcaagc

SEQ ID NO: 13 - DNA, 26 bp, Artificial Sequence
  Forward primer for amplification of the ITS2 locus in all yeast and mold/fungi
tttactttyaacaayggatctcttgg

SEQ ID NO: 14 - DNA, 26 bp, Artificial Sequence
  Reverse primer for amplification of the ITS2 locus in all yeast and mold/fungi
tttcttttcctccgcttattgatatg

SEQ ID NO: 15 - DNA, 28 bp, Artificial Sequence
  Forward primer for amplification of the ITS2 locus in Aspergillus spp
tttaaaggcagcggcggcaccgcgtccg

SEQ ID NO: 16 - DNA, 27 bp, Artificial Sequence
  Reverse primer for amplification of the ITS2 locus in Aspergillus spp
ttttcttttcctccgcttattgatatg

SEQ ID NO: 17 - DNA, 25 bp, Artificial Sequence
  Forward primer for amplification of the ITS1 locus in Cannabis/plant
tttgcaacagcagaacgacccgtga

SEQ ID NO: 18 - DNA, 25 bp, Artificial Sequence
  Reverse primer for amplification of the ITS1 locus in Cannabis/plant
tttcgataaacacgcatctcgattg

SEQ ID NO: 19 - DNA, 24 bp, Artificial Sequence
  Forward primer for amplification of the 16S rDNA HV3 locus in all bacteria
tttactgagacacggyccaractc

SEQ ID NO: 20 - DNA, 24 bp, Artificial Sequence
  Reverse primer for amplification of the 16S rDNA HV3 Locus in all bacteria
tttgtattaccgcggctgctggca

SEQ ID NO: 21 - DNA, 28 bp, Artificial Sequence
  Forward primer for amplification of the Stx1 locus in pathogenic Escherichia coli
tttatgtgacaggatttgttaacaggac

SEQ ID NO: 22 - DNA, 28 bp, Artificial Sequence
  Reverse primer for amplification of the Stx1 locus in pathogenic Escherichia coli
tttctgtcaccagacaatgtaaccgctg

SEQ ID NO: 23 - DNA, 24 bp, Artificial Sequence
  Forward primer for amplification of the Stx2 locus in pathogenic Escherichia coli
tttttgtcactgtcacagcagaag

SEQ ID NO: 24 - DNA, 25 bp, Artificial Sequence
  Reverse primer for amplification of the Stx2 locus in pathogenic Escherichia coli
tttgcgtcatcgtatacacaggagc

SEQ ID NO: 25 - DNA, 26 bp, Artificial Sequence
  Forward primer for amplification of the InvA locus in all Salmonella us
ttttatcgttattaccaaaggttcag

SEQ ID NO: 26 - DNA, 28 bp, Artificial Sequence
  Reverse primer for amplification of the InvA locus in all Salmonella
tttcctttccagtacgcttcgccgttcg

SEQ ID NO: 27 - DNA, 25 bp, Artificial Sequence
  Forward primer for amplification of the tuf locus in all Escherichia coli
tttgttgttaccggtcgtgtagaac

SEQ ID NO: 28 - DNA, 28 bp, Artificial Sequence
  Reverse primer for amplification of the tuf locus in all Escherichia coli
tttcttctgagtctctttgataccaacg

SEQ ID NO: 29 - DNA, 30 bp, Artificial Sequence
  Forward primer for amplification of the 16S rDNA HV3 locus in Enterobacteriaceae
ttatattgcacaatgggcgcaagcctgatg

SEQ ID NO: 30 - DNA, 25 bp, Artificial Sequence
  Reverse primer for amplification of the 16S rDNA HV3 locus in Enterobacteriaceae
ttttgtattaccgcggctgctggca

SEQ ID NO: 31 - DNA, 23 bp, Artificial Sequence
  Forward primer for amplification of the ITS2 locus in all yeast and mold/fungi
tttgcatcgatgaagarcgyagc

SEQ ID NO: 32 - DNA, 22 bp, Artificial Sequence
  Reverse primer for amplification of the ITS2 locus in all yeast and mold/fungi
tttcctccgcttattgatatgc

SEQ ID NO: 33 - DNA, 26 bp, Artificial Sequence
  Forward primer for amplification of the ITS2 locus in Aspergillus spp
tttcctcgagcgtatggggctttgtc

SEQ ID NO: 34 - DNA, 24 bp, Artificial Sequence
  Reverse primer for amplification of the ITS2 locus in Aspergillus spp
tttttcctccgcttattgatatgc

SEQ ID NO: 35 - DNA, 27 bp, Artificial Sequence
  Forward primer for amplification of the ITS1 locus in Cannabis/plant
tttcgtgaacacgttttaaacagcttg

SEQ ID NO: 36 - DNA, 24 bp, Artificial Sequence
  Reverse primer for amplification of the ITS1 locus in Cannabis/plant
tttccaccgcacgagccacgcgat

SEQ ID NO: 37 - DNA, 30 bp, Artificial Sequence
  Probe sequence for the 16S locus for total aerobic bacteria with high sensitivity
tttttttttcctacgggaggcagttttttt

SEQ ID NO: 38 - DNA, 30 bp, Artificial Sequence
  Probe sequence for the 16S locus for total aerobic bacteria with medium sensitivity
ttttttttccctacgggaggcatttttttt

SEQ ID NO: 39 - DNA, 30 bp, Artificial Sequence
  Probe sequence for the 16S locus for total aerobic bacteria with low sensitivity
tttattttccctacgggaggcttttatttt

SEQ ID NO: 40 - DNA, 30 bp, Artificial Sequence
  Probe sequence for the 16S locus in Enterobacteriaceae with low sensitivity
tttattctattgacgttacccatttatttt

SEQ ID NO: 41 - DNA, 30 bp, Artificial Sequence
  Probe sequence for the 16S locus in Enterobacteriaceae with medium sensitivity
ttttttctattgacgttacccgtttttttt

SEQ ID NO: 42 - DNA, 30 bp, Artificial Sequence
  Probe sequence for the 16S locus in Escherichia coli/Shigella
ttttctaatacctttgctcattgactcttt

SEQ ID NO: 43 - DNA, 30 bp, Artificial Sequence
  Probe sequence for the 16S locus in Escherichia coli/Shigella
ttttttaagggagtaaagttaatatttttt

SEQ ID NO: 44 - DNA, 30 bp, Artificial Sequence
  Probe sequence for the 16S locus in Escherichia coli/Shigella
ttttctcctttgctcattgacgttattttt

SEQ ID NO: 45 - DNA, 30 bp, Artificial Sequence
  Probe sequence for the 16S locus in Bacillus spp. Group 1
tttttcagttgaataagctggcactctttt

SEQ ID NO: 46 - DNA, 30 bp, Artificial Sequence
  Probe sequence for the 16S locus in Bacillus spp. Group 2
ttttttcaagtaccgttcgaatagtttttt

SEQ ID NO: 47 - DNA, 36 bp, Artificial Sequence
  Probe sequence for the 16S locus in bile-tolerant gram negative bacteria with high sensitivity
tttttctatgcagtcatgctgtgtgtrtgtcttttt

SEQ ID NO: 48 - DNA, 34 bp, Artificial Sequence
  Probe sequence for the 16S locus in bile-tolerant gram negative bacteria with medium sensitivity
tttttctatgcagccatgctgtgtgtrttttttt

SEQ ID NO: 49 - DNA, 34 bp, Artificial Sequence
  Probe sequence for the 16S locus in bile-tolerant gram negative bacteria with low sensitivity
tttttctatgcagtcatgctgcgtgtrttttttt

SEQ ID NO: 50 - DNA, 30 bp, Artificial Sequence
  Probe sequence for the 16S locus in Campylobacter spp.
ttttttatgacacttttcggagctcttttt

SEQ ID NO: 51 - DNA, 34 bp, Artificial Sequence
  Probe sequence for the 16S locus in Chromobacterium spp.
ttttattttcccgctggttaataccctttatttt

SEQ ID NO: 52 - DNA, 30 bp, Artificial Sequence
  Probe sequence for the 16S locus in Citrobacter spp. Gtoup 1
ttttttccttagccattgacgttatttttt

SEQ ID NO: 53 - DNA, 30 bp, Artificial Sequence
  Probe sequence for the 16S locus in Clostridium spp.
ttttctggamgataatgacggtacagtttt

SEQ ID NO: 54 - DNA, 30 bp, Artificial Sequence
  Probe sequence for the 16S locus in Coliform/Enterobacteriaceae
ttttttctattgacgttacccgcttttttt

SEQ ID NO: 55 - DNA, 30 bp, Artificial Sequence
  Probe sequence for the 16S locus in Aeromonas salmonicida/hydrophilia
tttttgcctaatacgtrtcaactgcttttt

SEQ ID NO: 56 - DNA, 30 bp, Artificial Sequence
  Probe sequence for the 16S locus in Aeromonas spp.
ttattttctgtgacgttactcgcttttatt

SEQ ID NO: 57 - DNA, 30 bp, Artificial Sequence
  Probe sequence for the 16S locus in Alkanindiges spp.
tttttaggctactgrtactaatatcttttt

SEQ ID NO: 58 - DNA, 34 bp, Artificial Sequence
  Probe sequence for the 16S locus in Bacillus pumilus
tttatttaagtgcragagtaactgctattttatt

SEQ ID NO: 59 - DNA, 34 bp, Artificial Sequence
  Probe sequence for bacterial etuf gene
ttttttccatcaaagttggtgaagaatctttttt

SEQ ID NO: 60 - DNA, 30 bp, Artificial Sequence
  Probe sequence for the 16S locus in Hafnia spp.
ttttttctaaccgcagtgattgatcttttt

SEQ ID NO: 61 - DNA, 34 bp, Artificial Sequence
  Probe sequence for bacterial InvA gene
tttttttattgatgccgatttgaaggcctttttt

SEQ ID NO: 62 - DNA, 30 bp, Artificial Sequence
  Probe sequence for the 16S locus in Klebsiella oxytoca
ttttttctaaccttattcattgatcttttt

SEQ ID NO: 63 - DNA, 30 bp, Artificial Sequence
  Probe sequence for the 16S locus in Klebsiella pneumoniae
ttttttctaaccttggcgattgatcttttt

SEQ ID NO: 64 - DNA, 34 bp, Artificial Sequence
  Probe sequence for the 16S locus in Legionella spp.
tttattctgataggttaagagctgatctttattt

SEQ ID NO: 65 - DNA, 30 bp, Artificial Sequence
  Probe sequence for the 16S locus in Listeria spp.
ttttctaagtactgttgttagagaattttt

SEQ ID NO: 66 - DNA, 30 bp, Artificial Sequence
  Probe sequence for the 16S locus in Panteoa agglomerans
ttttttaaccctgtcgattgacgccttttt

SEQ ID NO: 67 - DNA, 30 bp, Artificial Sequence
  Probe sequence for the 16S locus in Panteoa stewartii
ttttttaacctcatcaattgacgccttttt

SEQ ID NO: 68 - DNA, 30 bp, Artificial Sequence
  Probe sequence for the 16S locus in Pseudomonas aeruginosa
tttttgcagtaagttaataccttgtctttt

SEQ ID NO: 69 - DNA, 30 bp, Artificial Sequence
  Probe sequence for the 16S locus in Pseudomonas cannabina
tttttttacgtatctgttttgactcttttt

SEQ ID NO: 70 - DNA, 30 bp, Artificial Sequence
  Probe sequence for the 16S locus in Pseudomonas spp.
ttttttgttaccracagaataagcattttt

SEQ ID NO: 71 - DNA, 30 bp, Artificial Sequence
  Probe sequence for the 16S locus in Pseudomonas spp.
ttttttaagcactttaagttgggatttttt

SEQ ID NO: 72 - DNA, 34 bp, Artificial Sequence
  Probe sequence for the 16S locus in Pseudomonas spp.
tttattttaagcactttaagttgggattttattt

SEQ ID NO: 73 - DNA, 30 bp, Artificial Sequence
  Probe sequence for the 16S locus in Salmonella bongori
tttttttaataaccttgttgattgtttttt

SEQ ID NO: 74 - DNA, 30 bp, Artificial Sequence
  Probe sequence for the 16S locus in Salmonella enterica
tttttttgttgtggttaataaccgattttt

SEQ ID NO: 75 - DNA, 30 bp, Artificial Sequence
  Probe sequence for the 16S locus in Salmonella enterica
tttttttaaccgcagcaattgactcttttt

SEQ ID NO: 76 - DNA, 30 bp, Artificial Sequence
  Probe sequence for the 16S locus in Salmonella enterica
ttttttctgttaataaccgcagcttttttt

SEQ ID NO: 77 - DNA, 34 bp, Artificial Sequence
  Probe sequence for the 16S locus in Serratia spp.
tttattctgtgaacttaatacgttcatttttatt

SEQ ID NO: 78 - DNA, 34 bp, Artificial Sequence
  Probe sequence for the 16S locus in Staphylococcus aureus
tttattttcatatgtgtaagtaactgttttattt

SEQ ID NO: 79 - DNA, 30 bp, Artificial Sequence
  Probe sequence for the 16S locus in Staphylococcus aureus
ttttttcatatgtgtaagtaactgtttttt

SEQ ID NO: 80 - DNA, 34 bp, Artificial Sequence
  Probe sequence for the 16S locus in Streptomyces spp.
ttttattttaagaagcgagagtgacttttatttt

SEQ ID NO: 81 - DNA, 30 bp, Artificial Sequence
  Probe sequence for bacterial stx1 gene
ttttttctttccaggtacaacagctttttt

SEQ ID NO: 82 - DNA, 30 bp, Artificial Sequence
  Probe sequence for bacterial stx2 gene
ttttttgcactgtctgaaactgcctttttt

SEQ ID NO: 83 - DNA, 30 bp, Artificial Sequence
  Probe sequence for the 16S locus in Vibrio spp.
ttttttgaaggtggttaagctaattttttt

SEQ ID NO: 84 - DNA, 30 bp, Artificial Sequence
  Probe sequence for the 16S locus in Xanthamonas spp.
ttttttgttaatacccgattgttctttttt

SEQ ID NO: 85 - DNA, 30 bp, Artificial Sequence
  Probe sequence for the 16S locus in Ysernia pestis
tttttttgagtttaatacgctcaacttttt

SEQ ID NO: 86 - DNA, 38 bp, Artificial Sequence
  Probe sequence for the ITS2 locus in total yeast and mold with high sensitivity
ttttttttgaatcatcgartctttgaacgcattttttt

SEQ ID NO: 87 - DNA, 31 bp, Artificial Sequence
  Probe sequence for the ITS2 locus in total yeast and mold with low sensitivity
ttttttttgaatcatcgartctccttttttt

SEQ ID NO: 88 - DNA, 36 bp, Artificial Sequence
  Probe sequence for the ITS2 locus in total yeast and mold with medium sensitivity
ttttttttgaatcatcgartctttgaacgttttttt

SEQ ID NO: 89 - DNA, 34 bp, Artificial Sequence
  Probe sequence for the ITS2 locus in Alternaria spp.
ttttttcaaaggtctagcatccattaagtttttt

SEQ ID NO: 90 - DNA, 34 bp, Artificial Sequence
  Probe sequence for the ITS2 locus in Aspergillus flavus
ttttttcgcaaatcaatctttttccagtcttttt

SEQ ID NO: 91 - DNA, 40 bp, Artificial Sequence
  Probe sequence for the ITS2 locus in Aspergillus flavus
tttttttcttgccgaacgcaaatcaatctttttttttttt

SEQ ID NO: 92 - DNA, 34 bp, Artificial Sequence
  Probe sequence for the ITS2 locus in Aspergillus fumigatus
tttcttttcgacacccaactttatttccttattt

SEQ ID NO: 93 - DNA, 30 bp, Artificial Sequence
  Probe sequence for the ITS2 locus in Aspergillus fumigatus
tttttttgccagccgacacccattcttttt

SEQ ID NO: 94 - DNA, 30 bp, Artificial Sequence
  Probe sequence for the ITS2 locus in Aspergillus nidulans
ttttttggcgtctccaaccttacccttttt

SEQ ID NO: 95 - DNA, 30 bp, Artificial Sequence
  Probe sequence for the ITS2 locus in Aspergillus niger
ttttttcgacgttttccaaccatttctttt

SEQ ID NO: 96 - DNA, 34 bp, Artificial Sequence
  Probe sequence for the ITS2 locus in Aspergillus niger
ttttttttcgacgttttccaaccatttctttttt

SEQ ID NO: 97 - DNA, 30 bp, Artificial Sequence
  Probe sequence for the ITS2 locus in Aspergillus niger
tttttttcgccgacgttttccaattttttt

SEQ ID NO: 98 - DNA, 30 bp, Artificial Sequence
  Probe sequence for the ITS2 locus in Aspergillus terreus
tttttcgacgcatttatttgcaaccctttt

SEQ ID NO: 99 - DNA, 34 bp, Artificial Sequence
  Probe sequence for the ITS2 locus in Blumeria spp.
tttatttgccaaaamtccttaattgctctttttt

SEQ ID NO: 100 - DNA, 40 bp, Artificial Sequence
  Probe sequence for the ITS2 locus in Botrytis spp.
tttttttcatctctcgttacaggttctcggttcttttttt

SEQ ID NO: 101 - DNA, 34 bp, Artificial Sequence
  Probe sequence for the ITS2 locus in Candida albicans
tttttttttgaaagacggtagtggtaagtttttt

SEQ ID NO: 102 - DNA, 34 bp, Artificial Sequence
  Probe sequence for the ITS2 locus in Candida spp. Group 1
tttttttgtttggtgttgagcratacgtattttt

SEQ ID NO: 103 - DNA, 35 bp, Artificial Sequence
  Probe sequence for the ITS2 locus in Candida spp. Group 2
ttttactgtttggtaatgagtgatactctcatttt

SEQ ID NO: 104 - DNA, 34 bp, Artificial Sequence
  Probe sequence for the ITS2 locus in Chaetomium spp.
tttcttttggttccggccgttaaaccattttttt

SEQ ID NO: 105 - DNA, 34 bp, Artificial Sequence
  Probe sequence for the ITS2 locus in Cladosporium spp.
ttttttttgtggaaactattcgctaaagtttttt

SEQ ID NO: 106 - DNA, 34 bp, Artificial Sequence
  Probe sequence for the ITS2 locus in Erysiphe spp.
tttctttttacgattctcgcgacagagttttttt

SEQ ID NO: 107 - DNA, 34 bp, Artificial Sequence
  Probe sequence for the ITS2 locus in Fusarum oxysporum
tttttttctcgttactggtaatcgtcgttttttt

SEQ ID NO: 108 - DNA, 34 bp, Artificial Sequence
  Probe sequence for the ITS2 locus in Fusarium spp.
ttttttttaacacctcgcractggagattttttt

SEQ ID NO: 109 - DNA, 34 bp, Artificial Sequence
  Probe sequence for the ITS2 locus in Golovinomyces spp.
ttttttccgcttgccaatcaatccatctcttttt

SEQ ID NO: 110 - DNA, 34 bp, Artificial Sequence
  Probe sequence for the ITS2 locus in Histoplasma capsulatum
tttatttttgtcgagttccggtgcccttttattt

SEQ ID NO: 111 - DNA, 34 bp, Artificial Sequence
  Probe sequence for the ITS2 locus in Isaria spp.
tttatttttccgcggcgacctctgctctttattt

SEQ ID NO: 112 - DNA, 34 bp, Artificial Sequence
  Probe sequence for the ITS2 locus in Monocillium spp.
tttcttttgagcgacgacgggcccaattttcttt

SEQ ID NO: 113 - DNA, 30 bp, Artificial Sequence
  Probe sequence for the ITS2 locus in Mucor spp.
ttttctccawtgagyacgcctgtttctttt

SEQ ID NO: 114 - DNA, 34 bp, Artificial Sequence
  Probe sequence for the ITS2 locus in Myrothecium spp.
tttattttcggtggccatgccgttaaattttatt

SEQ ID NO: 115 - DNA, 34 bp, Artificial Sequence
  Probe sequence for the ITS2 locus in Oidiodendron spp.
tttttttgcgtagtacatctctcgctcatttttt

SEQ ID NO: 116 - DNA, 34 bp, Artificial Sequence
  Probe sequence for the ITS2 locus in Penicillium oxalicum
ttttttacaccatcaatcttaaccaggccttttt

SEQ ID NO: 117 - DNA, 34 bp, Artificial Sequence
  Probe sequence for the ITS2 locus in Penicillium paxilli
ttttttcccctcaatctttaaccaggcctttttt

SEQ ID NO: 118 - DNA, 34 bp, Artificial Sequence
  Probe sequence for the ITS2 locus in Penicillium spp.
ttttttcaacccaaatttttatccaggccttttt

SEQ ID NO: 119 - DNA, 34 bp, Artificial Sequence
  Probe sequence for the ITS2 locus in Phoma/Epicoccum ssp.
tttttttgcagtacatctcgcgctttgatttttt

SEQ ID NO: 120 - DNA, 34 bp, Artificial Sequence
  Probe sequence for the ITS2 locus in Podosphaera spp.
ttttttgacctgccaaaacccacataccattttt

SEQ ID NO: 121 - DNA, 34 bp, Artificial Sequence
  Probe sequence for the ITS2 locus in Podosphaera spp.
ttttttttagtcaygtatctcgcgacagtttttt

SEQ ID NO: 122 - DNA, 34 bp, Artificial Sequence
  Probe sequence for the ITS2 locus in Phythium oligandrum
ttttatttaaaggagacaacaccaatttttattt

SEQ ID NO: 123 - DNA, 34 bp, Artificial Sequence
  Probe sequence for the ITS2 locus in Rhodoturula spp.
ttttttctcgttcgtaatgcattagcactttttt

SEQ ID NO: 124 - DNA, 34 bp, Artificial Sequence
  Probe sequence for the ITS2 locus in Stachybotrys spp.
tttcttctgcatcggagctcagcgcgttttattt

SEQ ID NO: 125 - DNA, 34 bp, Artificial Sequence
  Probe sequence for the ITS2 locus in Trichoderma spp.
tttttcctcctgcgcagtagtttgcacatctttt

SEQ ID NO: 126 - DNA, 34 bp, Artificial Sequence
  Probe sequence for ITS1 locus in Cannabis species
ttttttaatctgcgccaaggaacaatattttttt

SEQ ID NO: 127 - DNA, 34 bp, Artificial Sequence
  Probe sequence for ITS1 locus in Cannabis species
tttttgcaatctgcgccaaggaacaatatttttt

SEQ ID NO: 128 - DNA, 34 bp, Artificial Sequence
  Probe sequence for ITS1 locus in Cannabis species
tttatttcttgcgccaaggaacaatattttattt

SEQ ID NO: 129 - DNA, 32 bp, Artificial Sequence
  Probe sequence for image calibration with high sensitivity
ttttctatgtatcgatgttgagaaattttttt

SEQ ID NO: 130 - DNA, 32 bp, Artificial Sequence
  Probe sequence for image calibration with low sensitivity
ttttctagatacttgtgtaagtgaattttttt

SEQ ID NO: 131 - DNA, 32 bp, Artificial Sequence
  Probe sequence for image calibration with medium sensitivity
ttttctaagtcatgttgttgaagaattttttt

SEQ ID NO: 132 - DNA, 34 bp, Artificial Sequence
  Probe sequence for negative control
ttttttctactacctatgctgattcactcttttt

SEQ ID NO: 133 - DNA, 23 bp, Artificial Sequence
  Forward primer for amplification of the ITS2 locus in all yeast and mold/fungi
tttgcatcgatgaagaacgcagc

SEQ ID NO: 134 - DNA, 23 bp, Artificial Sequence
  Reverse primer for amplification of the ITS2 locus in all yeast and mold/fungi
ttttcctccgcttattgatatgc

SEQ ID NO: 135 - DNA, 26 bp, Artificial Sequence
  Forward primer for amplification of the ITS2 locus in all yeast and mold/fungi
tttactttcaacaayggatctcttgg

SEQ ID NO: 136 - DNA, 38 bp, Artificial Sequence
  Probe sequence for total yeast and mold
ttttttttgcatcatagaaactttgtacgcattttttt

SEQ ID NO: 137 - DNA, 34 bp, Artificial Sequence
  Probe sequence for Golovinomyces species
tttatttaatcaatccatcatctcaagtcttttt

SEQ ID NO: 138 - DNA, 40 bp, Artificial Sequence
  Probe sequence for Mucor species
ttttttctccawtgagyacgcctgtttcagtatctttttt

SEQ ID NO: 139 - DNA, 34 bp, Artificial Sequence
  Probe sequence for Aspergillus terreus
ttttttacgcatttatttgcaacttgcctttttt

SEQ ID NO: 140 - DNA, 30 bp, Artificial Sequence
  Probe sequence for Podosphaera species
tttttcgtcccctaaacatagtggcttttt